SEQUENCE LISTING (31 sequences)

SEQ ID NO: 1 - DNA, 23 bp, Artificial
  An artificially synthesized primer sequence for RT-PCR
aaggattatgaggaggttggtgt

SEQ ID NO: 2 - DNA, 23 bp, Artificial
  An artificially synthesized primer sequence for RT-PCR
cttgggtctgtaacaaagcattc

SEQ ID NO: 3 - DNA, 23 bp, Artificial
  An artificially synthesized primer sequence for RT-PCR
ggtagctcagtcataaaacaccg

SEQ ID NO: 4 - DNA, 23 bp, Artificial
  An artificially synthesized primer sequence for RT-PCR
gtctctccatcatcctcactgtc

SEQ ID NO: 5 - protein, 17 aa, Artificial
  An artificially synthesized antigen polypeptide
CRAAGQSDSSVDPQQPF

SEQ ID NO: 6 - protein, 21 aa, Artificial
  An artificially synthesized antigen polypeptide
AEESEQERLLGGTRTPINDAS

SEQ ID NO: 7 - DNA, 19 bp, Artificial
  A target sequence for siRNA
ggagcacagcttccagcat

SEQ ID NO: 8 - DNA, 19 bp, Artificial
  A target sequence for siRNA
gcacgacagtcagcacaag

SEQ ID NO: 9 - DNA, 19 bp, Artificial
  A target sequence for siRNA
gtgacttcctctttatgga

SEQ ID NO: 10 - DNA, 19 bp, Artificial
  A target sequence for siRNA
gaagcagcacgacttcttc

SEQ ID NO: 11 - DNA, 3912 bp, Homo sapiens
  CDS (77)..(1192)
ggaagcgctcgcgcaggagaccccgggtgacggggcccggcgccgctaactggagcgaaccccagcgtccgccgacatggcctggaccaagtaccagctgttcctggccgggctcatgcttgttaccggctccatcaacacgctctcggcaaaatgggcggacaatttcatggccgagggctgtggagggagcaaggagcacagcttccagcatcccttcctccaggcagtgggcatgttcctgggagaattctcctgcctggctgccttctacctcctccgatgcagagctgcagggcaatcagactccagcgtagacccccagcagcccttcaaccctcttcttttcctgcccccagcgctctgtgacatgacagggaccagcctcatgtatgtggctctgaacatgaccagtgcctccagcttccagatgctgcggggtgcagtgatcatattcactggcctgttctcggtggccttcctgggccggaggctggtgctgagccagtggctgggcatcctagccaccatcgcggggctggtggtcgtgggcctggctgacctcctgagcaagcacgacagtcagcacaagctcagcgaagtgatcacaggggacctgttgatcatcatggcccagatcatcgttgccatccagatggtgctagaggagaagttcgtctacaaacacaatgtgcacccactgcgggcagttggcactgagggcctctttggctttgtgatcctctccctgctgctggtgcccatgtactacatccccgccggctccttcagcggaaaccctcgtgggacactggaggatgcattggacgccttctgccaggtgggccagcagccgctcattgccgtggcactgctgggcaacatcagcagcattgccttcttcaacttcgcaggcatcagcgtcaccaaggaactgagcgccaccacccgcatggtgttggacagcttgcgcaccgttgtcatctgggcactgagcctggcactgggctgggaggccttccatgcactgcagatccttggcttcctcatactccttataggcactgccctctacaatgggctacaccgtccgctgctgggccgcctgtccaggggccggcccctggcagaggagagcgagcaggagagactgctgggtggcacccgcactcccatcaatgatgccagctgaggttccctggaggcttctactgccacccgggtgctccttctccctgagactgaggccacacaggctggtgggccccgaatgccctatccccaaggcctcaccctgtcccctccctgcagaacccccagggcagctgctgccacagaagataacaacacccaagtcctctttttctcactaccacctgcagggtggtgttacccagcccccacaagcctgagtgcagtggcagacctcagctctctggacccctcctacagcactagagctaaatcatgaagttgaattgtaggaatttaccaccgtagtgtatctgaatcataaactagattatcatagttatctagtttatgagtcataagctagatttgattcttcaaagaagaaaagtcagtgagcaaatttaaaccagaactaagcattatattttctttttttttttttgagacagagtctcgctctgtcgcccaggctggagtgcagtggcacgatctcggcggctcactacaacctctgcctcccaggttcaaatgattctcctgcctcagcctcccgagtagctgggattacaggtgcataacacacctgggtaacttttatagagatggggtttcaccatgttggccaggctggtctcaaactcctgacctcaggtgatctgcctgcctcggcctcccaaagtgctgggattacaggcgtgagacaccacacccagccataactaagcattatgttttctaaaacttctaagatcctccctaaacccgtctggagacatgggttttagcccagactctgcctcaaactcattgtagctcccagcacattacccagttgctctgggccttggtgttatgctctgggaagtagatgttgatgctgtggccttagtgccttctggccccagcattccatgggcctgtgatcttgaccaacctgagaaaacagtaacagcccatccactggaaatacccctctgcccacagcaccacccttctgtgctgttttttttgttgtttgtttgtttcttctttttgagacagagtcttgctctgttgccacgctggagtgcagtggtgtgatctcggctcactgcaacctcccgctcccaggttcaagcaagtcccctgagtagctgggactacaggcgtgtgccaccacatccggctaattttttgcattctttaatagagacggggttttgccatgttggccaggatggtctcaatctcctgacctcatgatccacctgcctcagcctcccaaagtgctggggttacaggcatgagccaccacgcccagccttttttttttttttttttttttcttgagagacagggtcttgctctgtcacccaggcacagcttactgcagccttgaactcctgggatcaatcagtcctcctgcctcagcctcccgagtagctaagactaacaggtatgtaccaccatgcctggtttattgttttatttttttggcagagatgggtctcactgtgttgcccaggctgatctcaaactcctggcctcaagcgatcctcccatctcagcctcccaaagtgctgggattacagacctgagccaccacacctgggcaacagagtgaaacctgtccctgttttcctgctcttactctcacctctgaggcctcctctgcctggaagagattacagggaaattccaggcagcccttgtcaattgtttttatgaattctttacctgttccttttaaagacaaggaaactgaggcccaaagttctaagttgtttggcaaatggagtctcctaccctcagctcctgcaaggacctgggggacccccaggtccagcagccacatgattctgcagcagacagggacctagagcacatctggatctcagccccacccctggcaacctgcctgcctagagaactcccaagatgacagactaagtaggattctgccatttagaataattctggtatcctgggcgttgcgttaagttgcttaactttcattctgtcttacgatagtcttcagaggtgggaacagatgaagaaaccatgccccagagaaggttaagtgacttcctctttatggagccagtgttccaacctaggtttgcctgataccagacctgtggccccacctcccatgcaggtctctgtggggtctttgggatggatctcctagggctgggctggaagcctcatgtactgttgtcttctaggtaaccaccctgaagagaaggggtggcatcaggaaccgcagggaaccaagcagcctttggtccagtgctgctccttggaacagttgagtgtggcctcaaaccattctcctgggtagctcagtcataaaacaccgaatgcctgcctcaaaatagcctctgagggaagggagctgttgacaagtgaagccctcaggttgtgtgggattccagcaggttattacagcttgtggggggagggagggtccattccaccagctttgaatcccagggtcccctcagcttcactgggccacagtgtctgtcccaggaagaagcaaatggacagtgaggatgatggagagactacagctgccttgtctaaaacctgggatcttaaaagtgttattttggcttttattttttaaaaaaagaaaaggaaaattggcaattgtattaatccattctcgaattgctgtaaagaaatacctgagactgggtaatttaatttataaagaaaagaggtttaattggc

SEQ ID NO: 12 - protein, 371 aa, Homo sapiens
MAWTKYQLFLAGLMLVTGSINTLSAKWADNFMAEGCGGSKEHSFQHPFLQAVGMFLGEFSCLAAFYLLRCRAAGQSDSSVDPQQPFNPLLFLPPALCDMTGTSLMYVALNMTSASSFQMLRGAVIIFTGLFSVAFLGRRLVLSQWLGILATIAGLVVVGLADLLSKHDSQHKLSEVITGDLLIIMAQIIVAIQMVLEEKFVYKHNVHPLRAVGTEGLFGFVILSLLLVPMYYIPAGSFSGNPRGTLEDALDAFCQVGQQPLIAVALLGNISSIAFFNFAGISVTKELSATTRMVLDSLRTVVIWALSLALGWEAFHALQILGFLILLIGTALYNGLHRPLLGRLSRGRPLAEESEQERLLGGTRTPINDAS

SEQ ID NO: 13 - DNA, 51 bp, Artificial
  An artificially synthesized oligonucleotide for siRNA
caccggagcacagcttccagcatttcaagagaatgctggaagctgtgctcc

SEQ ID NO: 14 - DNA, 51 bp, Artificial
  An artificially synthesized oligonucleotide for siRNA
aaaaggagcacagcttccagcattctcttgaaatgctggaagctgtgctcc

SEQ ID NO: 15 - DNA, 47 bp, Artificial
  siRNA hairpin design
ggagcacagcttccagcatttcaagagaatgctggaagctgtgctcc

SEQ ID NO: 16 - DNA, 51 bp, Artificial
  An artificially synthesized oligonucleotide for siRNA
caccgcacgacagtcagcacaagttcaagagacttgtgctgactgtcgtgc

SEQ ID NO: 17 - DNA, 51 bp, Artificial
  An artificially synthesized oligonucleotide for siRNA
aaaagcacgacagtcagcacaagtctcttgaacttgtgctgactgtcgtgc

SEQ ID NO: 18 - DNA, 47 bp, Artificial
  siRNA hairpin design
gcacgacagtcagcacaagttcaagagacttgtgctgactgtcgtgc

SEQ ID NO: 19 - DNA, 51 bp, Artificial
  An artificially synthesized oligonucleotide for siRNA
caccgtgacttcctctttatggattcaagagatccataaagaggaagtcac

SEQ ID NO: 20 - DNA, 51 bp, Artificial
  An artificially synthesized oligonucleotide for siRNA
aaaagtgacttcctctttatggatctcttgaatccataaagaggaagtcac

SEQ ID NO: 21 - DNA, 47 bp, Artificial
  siRNA hairpin design
gtgacttcctctttatggattcaagagatccataaagaggaagtcac

SEQ ID NO: 22 - DNA, 51 bp, Artificial
  An artificially synthesized oligonucleotide for siRNA
caccgaagcagcacgacttcttcttcaagagagaagaagtcgtgctgcttc

SEQ ID NO: 23 - DNA, 51 bp, Artificial
  An artificially synthesized oligonucleotide for siRNA
aaaagaagcagcacgacttcttctctcttgaagaagaagtcgtgctgcttc

SEQ ID NO: 24 - DNA, 47 bp, Artificial
  siRNA hairpin design
gaagcagcacgacttcttcttcaagagagaagaagtcgtgctgcttc

SEQ ID NO: 25 - DNA, 1284 bp, Homo sapiens
agctccggctccccctatataaatcggccatttgcttcgctccgccccgcagcgccggagtcaaagccggttcccggcccagtcccgtcctgcagcagtctgcctcctctttcaacatgacagatgccgctgtgtccttcgccaaggacttcctggcaggtggagtggccgcagccatctccaagacggcggtagcgcccatcgagcgggtcaagctgctgctgcaggtgcagcatgccagcaagcagatcactgcagataagcaatacaaaggcattatagactgcgtggtccgtattcccaaggagcagggagttctgtccttctggcgcggtaacctggccaatgtcatcagatacttccccacccaggctcttaacttcgccttcaaagataaatacaagcagatcttcctgggtggtgtggacaagagaacccagttttggctctactttgcagggaatctggcatcgggtggtgccgcaggggccacatccctgtgttttgtgtaccctcttgattttgcccgtacccgtctagcagctgatgtgggtaaagctggagctgaaagggaattccgaggcctcggtgactgcctggttaagatctacaaatctgatgggattaagggcctgtaccaaggctttaacgtgtctgtgcagggtattatcatctaccgagccgcctacttcggtatctatgacactgcaaagggaatgcttccggatcccaagaacactcacatcgtcatcagctggatgatcgcacagactgtcactgctgttgccgggttgacttcctatccatttgacactgttcgccgccgcatgatgatgcagtcagggcgcaaaggaactgacatcatgtacacaggcacgcttgactgctggcggaagattgctcgtgatgaaggaggcaaagcttttttcaagggtgcatggtccaatgttctcagaggcatgggtggtgcttttgtgcttgtcttgtatgatgaaatcaagaagtacacataagttatttcctaggatttttccccctgtgaacaggcatgttgtattatataacatatcttgagcattcttgacagactcctggctgtcagtttctcagtggcaactatttactggttgaaaatgggaagcaataatattcatctgaccagttttctcttaaagccatttccatgatgatgatgatgggactcaattgtattttttatttcagtcactcctgataaataacaaatttggagaaataaaaatatctaaaataaattttgtctgc

SEQ ID NO: 26 - protein, 298 aa, Homo sapiens
MTDAAVSFAKDFLAGGVAAAISKTAVAPIERVKLLLQVQHASKQITADKQYKGIIDCVVRIPKEQGVLSFWRGNLANVIRYFPTQALNFAFKDKYKQIFLGGVDKRTQFWLYFAGNLASGGAAGATSLCFVYPLDFARTRLAADVGKAGAEREFRGLGDCLVKIYKSDGIKGLYQGFNVSVQGIIIYRAAYFGIYDTAKGMLPDPKNTHIVISWMIAQTVTAVAGLTSYPFDTVRRRMMMQSGRKGTDIMYTGTLDCWRKIARDEGGKAFFKGAWSNVLRGMGGAFVLVLYDEIKKYT

SEQ ID NO: 27 - DNA, 34 bp, Artificial
  An artificially synthesized primer sequence for PCR
atttgaggaagatcatggcctggaccaagtacca

SEQ ID NO: 28 - DNA, 28 bp, Artificial
  An artificially synthesized primer sequence for PCR
ccgctcgaggctggcatcattgatggga

SEQ ID NO: 29 - DNA, 34 bp, Artificial
  An artificially synthesized primer sequence for PCR
attcgcggccgctcatgacagatgccgctgtgtc

SEQ ID NO: 30 - DNA, 28 bp, Artificial
  An artificially synthesized primer sequence for PCR
ccgctcgagtgtgtacttcttgatttca

SEQ ID NO: 31 - DNA, 18 bp, Artificial
  A target sequence for siRNA
gcagatcactgcagataa

(19) United States
(12) Patent Application Publication
(10) Pub. No.: US 2011/0098339 A1
(43) Pub. Date: Apr. 28, 2011

(54) C2ORF18 AS TARGET GENE FOR CANCER THERAPY AND DIAGNOSIS

(75) Inventors: Yusuke Nakamura, Tokyo (JP); Hidewaki Nakagawa, Tokyo (JP); Shuichi Nakatsuru, Kanagawa (JP)

(73) Assignee: Oncotherapy Science, Inc., Kanagawa (JP)

(21) Appl. No.: 12/921,766

(22) PCT Filed: Mar. 10, 2009
(86) PCT No.: PCT/JP2009/001057

§ 371 Date: Dec. 16, 2010

Related U.S. Application Data
(63) Continuation of application No. 61/036,035, filed on Mar. 12, 2008.

(51) Int. Cl. A61K 31/713; G01N 33/68; C07H 21/00; C12N 15/63; A61P 35/00

(52) U.S. Cl. 514/44 A; 436/501; 514/44 R; 536/24.5; 435/320.1

(57) ABSTRACT

Described herein are objective methods for detecting or diagnosing a predisposition to developing cancer, particularly pancreatic cancer. In one embodiment, the diagnostic method involves the step of determining an expression level of C2orf18 using anti-C2orf18 antibody. The present invention further provides methods of screening for therapeutic agents useful in the treatment of a C2orf18-associated disease, such as a cancer, e.g. pancreatic cancer, methods of inhibiting the cell growth and treating or alleviating their symptom. The invention also features products, such as polynucleotides, polypeptides, and vectors double-stranded molecules, antibodies, vectors and compositions composed thereof.

### PRIORITY

This application claims the benefit of U.S. Provisional Application Ser. No. 61/036,035, filed Mar. 12, 2008, the contents of which are hereby incorporated by reference in their entirety.

### TECHNICAL FIELD

1. Technical Field

The present invention relates to methods of detecting and diagnosing the presence of or a predisposition for developing cancer, particularly pancreatic cancer. The present invention also relates to methods of treating and preventing cancer, particularly pancreatic cancer. In particular, the present invention relates to C2orf18, a gene that is specifically up-regulated in cancer, and the use thereof as therapeutic target and diagnostic marker for cancer.

2. Background Art

Cancer is a leading cause of death and millions of people die from cancer every year in the world. Especially, pancreatic cancer has one of the highest mortality rates of any malignancy, and the 5-year-survival rate of patients is 4%. Approximately 28,000 patients are diagnosed with pancreatic cancer each year, and nearly all patients will die of their disease (Greenlee, R. T., et al., (2001) CA Cancer J Clin, 51: 15-36). The poor prognosis of this malignancy is a result of the difficulty of early diagnosis and poor response to current therapeutic methods (Greenlee, R. T., et al. (2001) CA Cancer J Clin, Ji: 15-36, Klinkenbijl, J. H., et al. (1999) Ann Surg, 230: 776-82; discussion 782-4.).

Pancreatic ductal adenocarcinoma (PDAC) is the fourth leading cause of cancer death in the western world and has one of the highest mortality rates among the common malignancies, with a 5-year patient survival rate of only 5% (DiMagno E P, et al. Gastroenterology. 1999 December; 117(6): 1464-84.1, Wray C J, et al. Gastroenterology. 2005 May; 128(6):1626-41.2). Approximately 37,170 new patients are expected to be diagnosed with pancreatic cancer in 2007 and nearly 33,370 will die of this disease in the United States (Jemal A, et al. CA Cancer J Clin. 2007 January-February; 57(1):43-66.3). Currently, the only curative treatment for PDACs is surgical resection. However, since the majority of PDAC patients are diagnosed at a very advanced stage, only 15%-20% of patients are candidates for surgical resection at the time of diagnosis. Of those who undergo the potentially curative surgery, most patients eventually relapse and die of their disease (Wray C J, et al. Gastroenterology. 2005 May; 128(6): 1626-41.2). Some approaches involving the combination of surgery with chemotherapy, including gemcitabine or 5-FU, with or without radiation, can improve patients'quality of life (DiMagno E P, et al. Gastroenterology. 1999 December; 117(6):1464-84.1, Wray C J, et al. Gastroenterology. 2005 May; 128(6):1626-41.2). However, such treatments have a very limited effect on the long-term survival of PDAC patients due to its extremely aggressive and chemo-resistant nature.

To overcome this gloomy situation, development of novel molecular therapies for PDAC through identification of molecular targets is an urgent issue now. Previously, precise expression profiles of PDAC cells have been generated using genome-wide cDNA microarray in combination with laser microdissection to obtain pure populations of cancer cells for testing (See Nakamura T, et al. Oncogene. 2004 Mar. 25; 23(13):2385-400, incorporated by reference.).

Mitochondria are vital for cellular bioenergetics and play a central role in determining the apoptotic process (Taniuchi K, et al. Cancer Res. 2005 Jan. 1; 65(1):105-12.). Cancer-associated changes in cellular metabolism influence mitochondrial function (Warburg effect, Galluzzi L, et al. Oncogene. 2006 Aug. 7; 25(34):4812-30.) and may be involved with cancer viability, especially in the hypotoxic condition. Furthermore, the invalidation of apoptosis has been linked to an inhibition of mitochondrial outer membrane permeabilization (MOMP). On theoretical grounds, it is tempting to develop specific therapeutic interventions that target the mitochondrial protein (Taniuchi K, et al. Cancer Res. 2005 Jan. 1; 65(1):105-12.). A variety of experimental therapeutic agents can directly target mitochondria, and thereby induce apoptosis. Examples of such agents include those designed to mimic the Bc1-2 homology domain 3 of Bc1-2-like proteins (Walensky L D, et al. Science. 2004 Sep. 3; 305(5689):1466-70.), the peripheral-type benzodiazepine receptor ligands (Decaudin D, et al. Cancer Res. 2002 Mar. 1; 62(5):1388-93.), and ampholytic cations (Ellerby H M, et al. Nat Med. 1999 September; 5(9):1032-8.). Such MOMP inducers or facilitators can induce apoptosis by themselves or facilitate apoptosis induction in combination therapies, and thereby negate any chemoresistance against DNA-damaging agents (Taniuchi K, et al. Cancer Res. 2005 Jan. 1; 65(1):105-12.).

The present invention addresses the need in the art for an improved pancreatic cancer diagnosis and therapy through the discovery of a new gene target, C2orf 18 (chromosome 2 open reading frame 18), that is specifically up-regulated in pancreatic cancer cells.

### DISCLOSURE OF INVENTION

### Summary of the Invention

In view of the foregoing, it is an object of the present invention to provide novel methods for detecting, diagnosing, prognosing monitoring, and/or treating cancer, particularly pancreatic cancer. In the course of the present invention, one novel gene C2orf18, was identified through the microarray analysis as specifically overexpressed in pancreatic cancer cells (specifically, PDAC cells) and its overexpression in the cancer cells was validated by RT-PCR and immunohistochemical analysis. Since it is restrictively expressed in adult normal organs, C2orf18 may be an appropriate and promising molecular target for a novel therapeutic approach having minimal adverse effect. Functionally, knockdown of endogenous C2orf18 by siRNA in pancreas cancer cell lines results in drastic suppression of pancreatic cancer cell growth, suggesting its essential role in maintaining the viability of pancreatic cancer cells. Results of immunocytochemical analysis and cell fractionation followed by western blot analysis suggest that C2orf18 is localized in mitochondria, indicating that C2orf18 may be involved with apoptosis or energy homeostasis in cancer cells.

Accordingly, it is an object of the present invention to provide methods of detecting or diagnosing cancer, determining a predisposition for developing cancer, and/or monitoring the course of a treatment for cancer, particularly pancreatic cancer, in a subject by determining an expression level of C2orf18 in a subject-derived biological sample, such as biopsy. An increase of the level of expression of C2orf18 as compared to a normal control level indicates that the subject suffers from or is at risk of developing cancer, particularly PDAC. In the methods of the present invention, the C2orf 18 gene can be detected by appropriate probes or the C2orf 18 protein can be detected by anti-C2orf 18 antibody.

The present invention further provides methods of identifying an agent that inhibits the expression of the C2orf18 gene or the activity of its gene product. As discussed in greater detail herein, the present invention relates to the discovery of an interaction between C2orf 18 and ANT2 and its involvement in maintenance of the mitochondrial membrane potential as well as apoptosis. Accordingly, it is an object of the present invention to provide methods of identifying an agent that inhibits the interaction between C2orf18 and ANT2. It is a further object of the present invention to provide methods of identifying a candidate agent for treating or preventing a C2orf18-associated disease, such as cancer, e.g. pancreatic cancer, more particularly PDAC, or a candidate agent for inhibiting cell growth under these diseased conditions. The methods of the present invention can be carried out either in vitro or in vivo. A decrease in the expression level of a C2orf18 gene and/or a biological activity of its gene product in the presence of a test agent as compared to that in the absence of the test agent indicates that the test agent is an inhibitor of C2orf18 and may be used to inhibit the growth of cells over-expressing C2orf18, such as cancerous cell, e.g. pancreatic cancer cell, more particularly PDAC. The candidate agent may be used to reduce a symptom of pancreatic cancer, particularly PDAC, by inhibiting the growth thereof.

It is yet a further object of the present invention to provide methods for inhibiting the growth of cancerous cells over-expressing C2orf18 by administering an agent that inhibits expression of the C2orf18 gene and a function of its gene product, the C2orf18 protein. Preferably, the agent is an inhibitory nucleic acid (e.g., an antisense, ribozyme, double stranded molecule). The agent may also be a nucleic acid molecule or vector for providing double-stranded molecule. Expression of the gene may be inhibited by introducing a double-stranded molecule into the target cell in an amount sufficient to inhibit expression of the C2orf18 gene. The present invention also provides methods for inhibiting the growth of cancerous cells over-expressing C2orf18 in a subject, as well as treating or preventing methods for the subjects suffering from cancer, particularly pancreatic cancer. In another aspect, the present invention relates to a pharmaceutical composition for treating or preventing cancer, particularly pancreatic cancer, that includes as an active ingredient double-stranded molecules or vectors encoding such in combination with a suitable pharmaceutically acceptable carrier. The double-stranded molecules of the present invention inhibit expression of the C2orf18 gene and inhibit the growth of cancerous cells over-expressing C2orf18 when introduced thereinto. For example, such molecules may be designed to target a sequence corresponding to the portion of SEQ ID NO: 11 extending between nucleotide residues 196 and 214 or nucleotide residues 574 and 592. The double-stranded molecules of the present invention include a sense strand and an antisense strand, wherein the sense strand includes a sequence containing the target sequence, and wherein the antisense strand includes a sequence which is complementary to the sense strand. The sense and the antisense strands of the molecule hybridize to each other to form a double-stranded molecule of the present invention.

It is a further object of the present invention to provide an antibody against C2orf18. The antibody is raised using as an antigen a polypeptide having an amino acid sequence of CRAAGQSDSSVDPQQPF (SEQ ID NO: 5) and/or AEESEQERLLGGTRTPINDAS (SEQ ID NO: 6). These antibodies are useful in the context of immunological stain assays for diagnosing cancer, particularly pancreatic cancer. In another aspect, the present invention provides a detection reagent or kit for detecting, diagnosing, or prognosing cancer, particularly pancreatic cancer, that includes anti-C2orf18 antibody. In the present invention, the peptides having an amino acid sequence of CRAAGQSDSSVDPQQPF (SEQ ID NO: 5) and/or AEESEQERLLGGTRTPINDAS (SEQ ID NO: 6) are particularly useful for preparing an anti-C2orf18 antibody. Accordingly, it is an object of the present invention to provide these peptide as well as the methods of preparing an anti-C2orf18 antibody.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention.

These and other objects and features of the invention will become more fully apparent when the following detailed description is read in conjunction with the accompanying figures and examples. However, it is to be understood that both the foregoing summary of the invention and the following detailed description merely set forth preferred embodiments, and are not restrictive of the invention or other alternate embodiments of the invention. In particular, while the invention is described herein with reference to a number of specific embodiments, it will be appreciated that the description is illustrative of the invention and should not be constructed as limiting the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

### BRIEF DESCRIPTION OF THE DRAWINGS

Various aspects and applications of the present invention will become apparent to the skilled artisan upon consideration of the brief description of the figures and the detailed description of the present invention and its preferred embodiments which follows:

FIG. 1 “C2orf18 over-expression in PDAC cells” Part (A) depicts the results of RT-PCR for mRNA expressions of C2orf18 and TUBA in the microdissected PDAC cells (1-9), comparing with normal pancreatic ductal epithelial cells (NPD) which were also microdissected and vital adult organs. Panc: normal pancreas, BM: bone marrow.Part (B) depicts the results of Northern blot analysis, which revealed that C2orf18 was expressed faintly at the prostate, thyroid, adrenal gland tissues, highly in almost all pancreatic cancer cell lines, but had very limited expression in vital organs.Part (C) depicts the results of Western blot analysis using anti-C2orf18 antibody (lot#192 and lot#193) purified from the sera of two rabbits detected endogenous C2orf18 in PDAC cell lines. PAPM expression in PDAC cell lines was higher than that in normal cell lines (NIH3T3, HEK-293, and COS7). beta-Actin was served as loading control.Part (D) depicts the results of an immunohistochemical study using anti-C2orf18 antibody, wherein intense staining was observed in PDAC cells (C1×200, C2×400, C3×400). Strong positive staining of C2orf18 was observed at the cytoplasm of PDAC cells. In normal pancreatic tissue, acinar cells and normal ductal epithelium cells showed no staining (N, x400).

FIG. 2 “Effect of C2orf18-siRNAs on growth of PDAC cells” Part (A) depicts the knockdown effect of siRNA on C2orf18 in PDAC cell lines, MIA-PaCa2 (left) and Panc-1 (right). Semi-quantitative RT-PCR was performed using cells transfected with each of siRNA-expressing vectors to C2orf18 (#196, #574, and #3254) as well as a negative control vectors siEGFP), which confirmed the knockdown effect by #196 and #574, but not by siEGFP and #3254. beta2-MG was used to quantify RNAs.Part (B) depicts the results of a colony formation assay of MIA-PaCa2 (left) and Panc-1 (right) cells transfected with each of indicated siRNA-expressing vectors to C2orf18 (#196, #574, and #3254) and a negative control vector (siEGFP). Cells were visualized with 0.1% crystal violet staining after 14-day incubation with Geneticin Part (C) depicts the results of an MTT assay using each of MIA-PaCa2 (left) and Panc-1 (right) cells transfected with indicated siRNA-expressing vectors to C2orf18 (#196, #574, and #3254) and a negative control vector (siEGFP). Each average is plotted with error bars indicating SD (standard deviation) after 14-day incubation with Geneticin. ABS on Y-axis means absorbance at 490 nm, and at 630 nm as reference, measured with a microplate reader. These experiments were carried out in triplicate. ** Means P value of <0.01 (Students't-test).

FIG. 3 “Localization of C2orf 18 in PDAC cells” Part (A) depicts the results of immunocytochemical analysis using an anti-C2orf 18 antibody, wherein positive signals (green) were detected as vesicular patterns in the cytoplasm of PDAC cells (upper left panel). These signals for anti-C2orf18 antibody (green) were partially merged with MitoTracker signals (red: upper right panel). These signals for anti-C2orf18 antibody were disappeared in knocking down C2orf 18 by siRNA (lower right panel), while they retained as vesicular patterns in the cytoplasm by treating with the control siRNA (siEGFP) (lower left panel).Part (B) depicts the results of Western blot analysis using an anti-C2orf18 antibody, wherein it was demonstrated that C2orf18 was localized in the cytoplasm and only in the mitochondrial fraction. An anti-mitofilin antibody was used to detect the mitochondrial fraction.Part (C) depicts the results of an immunoprecipitation assay followed by mass spectrometry, wherein ANT2 was identified as a candidate of C2orf18-interacting proteins. C2orf18-HA expression vector and/or ANT2-Flag expression vector were co-transfected into COS7 cells. Protein complexes containing C2orf18-HA or ANT2-Flag were immunoprecipitated from cell extracts by anti-HA antibody (left panel) or anti-Flag antibody (right panel), respectively. Western blot using anti-Flag antibody indicated that ANT2-Flag was co-immunoprecipitated with C2orf18-HA when the both expression vectors were co-transfected (arrowed in left panel). Western blot using anti-HA antibody indicated that C2orf 18-HA was co-immunoprecipitated with ANT2-Flag when the both expression vectors were co-expressed (arrowed in right panel).

FIG. 4 “C2orf18/ANT2BP was involved with the mitochondrial membrane potential (delta psi m) and apoptosis” Part (A) depicts the results of transfection studies, wherein PDAC cells, KLM-1 were transfected with ANT2 siRNA, C2orf18/ANT2BP siRNA, or siEGFP (as a control) and collected 48h after transfection. Western blot analysis using anti-C2orf18/ANT2BP antibody confirmed knockdown effect of C2orf18/ANT2BP siRNA on KLM-1 cell.Part (B) depicts the results of a fluorescence assay, wherein the cells were incubated with Rhodamine-123 and PI, and fluorescence was measured by FACS analysis. Rhodamine-123 (Rh123) intensity at X-axis reflects delta psi m and PI (propidium iodide) permeability at X-axis reflects the cell membrane destruction in dead cells. The low level of Rh123 intensity and negative-permeability of PI indicate the early apoptotic cells where mitochondrial delta psi m is decreased but apoptosis does not finish completely, while the low level of Rh123 intensity and positive-permeability of PI indicate dead cells. The numbers of the cells showing low delta psi m and negative-permeability of PI were increased when ANTBP2 (25%) or ANT2 (26%) was knocked down, comparing with the control (siEGFP, 19%).Part (C) depicts the results of a TUNEL assay demonstrating that knockdown of C2orf18 in Panc-1 cells increased the number of apoptosis cells (TUNEL-positive cells indicated by green) comparing with siEGFP-transfected cells. The permeabilized cells treated with DNase I were prepared as positive controls for TUNEL assay.Part (D) depicts the number of TUNEL-positive cells counted by flow cytometry. In histogram plot, X-axis reflects the intensity of green signals of TUNEL-positive cells. Shift to the right of histogram indicates that knockdown of C2orf18 increased the number of TUNEL-positive cells comparing with siEGFP-transfected cells.

### DISCLOSURE OF THE INVENTION

Although methods and materials similar or equivalent to those described herein can be used in the practice or testing of embodiments of the present invention, the preferred methods and materials are now described. However, it is to be understood that the present invention is not limited to the specific methodologies and protocols herein described, as these may vary in accordance with routine experimentation and optimization. It is also to be understood that the terminology used in the description is for the purpose of describing the particular versions or embodiments only, and is not intended to limit the scope of the present invention.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the present invention, the following definitions apply. Additional definitions are interspersed in the subsequent text, where applicable.

### DEFINITIONS

The words “a”, “an”, and “the” as used herein mean “at least one” unless otherwise specifically indicated.

As used herein, the term “organism” refers to any living entity composed of at least one cell. A living organism can be as simple as, for example, a single eukaryotic cell or as complex as a mammal, including a human being.

As used herein, the term “biological sample” refers to a whole organism or a subset of its tissues, cells or component parts (e.g., body fluids, including but not limited to blood, mucus, lymphatic fluid, synovial fluid, cerebrospinal fluid, saliva, amniotic fluid, amniotic cord blood, urine, vaginal fluid and semen). The term “biological sample” further refers to a homogenate, lysate, extract, cell culture or tissue culture prepared from a whole organism or a subset of its cells, tissues or component parts, or a fraction or portion thereof. Lastly, “biological sample” refers to a medium, such as a nutrient broth or gel in which an organism has been propagated, which contains cellular components, such as proteins or polynucleotides.

In the context of the present invention, the subject-derived sample may be any tissue obtained from a test subject, e.g., a subject known to or suspected of having cancer, more particularly pancreatic cancer. For example, the tissue can contain epithelial cells. More particularly, the tissue can be epithelial cells from pancreatic cancer or PDAC.

The terms “isolated” and “purified” when used herein in relation to a substance (e.g., polypeptide, antibody, polynucleotide, etc.) indicate that the substance is substantially free from at least one substance that may else be included in the natural source. Thus, an “isolated” or “purified” antibody refers to an antibody that is substantially free of cellular material such as carbohydrate, lipid, or other contaminating proteins from the cell or tissue source from which the protein (antibody) is derived, or substantially free of chemical precursors or other chemicals when chemically synthesized. The term “substantially free of cellular material” includes preparations of a polypeptide in which the polypeptide is separated from cellular components of the cells from which it is isolated or recombinantly produced. Thus, a polypeptide that is substantially free of cellular material includes preparations of polypeptide having less than about 30%, 20%, 10%, 5%, 2% or 1% (by dry weight) of heterologous protein (also referred to herein as a “contaminating protein”). When the polypeptide is recombinantly produced, it is also preferably substantially free of culture medium, which includes preparations of polypeptide with culture medium less than about 20%, 10%, or 5% of the volume of the protein preparation. When the polypeptide is produced by chemical synthesis, it is preferably substantially free of chemical precursors or other chemicals, which includes preparations of polypeptide with chemical precursors or other chemicals involved in the synthesis of the protein less than about 30%, 20%, 10%, 5%, 2%, or 1% (by dry weight) of the volume of the protein preparation. That a particular protein preparation contains an isolated or purified polypeptide can be shown, for example, by the appearance of a single band following sodium dodecyl sulfate (SDS)-polyacrylamide gel electrophoresis of the protein preparation and Coomassie Brilliant Blue staining or the like of the gel. In a preferred embodiment, antibodies of the present invention are isolated or purified.

An “isolated” or “purified” nucleic acid molecule, such as a cDNA molecule, can be substantially free of other cellular material, or culture medium when produced by recombinant techniques, or substantially free of chemical precursors or other chemicals when chemically synthesized. In a preferred embodiment, nucleic acid molecules encoding antibodies of the present invention are isolated or purified.

The terms “polypeptide”, “peptide”, and “protein” are used interchangeably herein to refer to a polymer of amino acid residues. The terms apply to amino acid polymers in which one or more amino acid residue is a modified residue, or a non-naturally occurring residue, such as an artificial chemical mimetic of a corresponding naturally occurring amino acid, as well as to naturally occurring amino acid polymers.

The term “amino acid” refers to naturally occurring and synthetic amino acids, as well as amino acid analogs and amino acid mimetics that similarly functions to the naturally occurring amino acids. Naturally occurring amino acids are those encoded by the genetic code, as well as those modified after translation in cells (e.g., hydroxyyproline, gamma-carboxyglutamate, and O-phosphoserine). The phrase “amino acid analog” refers to compounds that have the same basic chemical structure (an alpha carbon bound to a hydrogen, a carboxy group, an amino group, and an R group) as a naturally occurring amino acid but have a modified R group or modified backbones (e.g., homoserine, norleucine, methionine, sulfoxide, methionine methyl sulfonium). The phrase “amino acid mimetic” refers to chemical compounds that have different structures but similar functions to general amino acids.

Amino acids may be referred to herein by their commonly known three letter symbols or the one-letter symbols recommended by the IUPAC-IUB Biochemical Nomenclature Commission.

In the context of the present invention, the term “several”, for example as applied to amino acid additions, deletions, and/or substitutions means 3-7, preferably 3-5, more preferably 3-4, even more preferably 3 amino acid residues.

The terms “polynucleotides”, “oligonucleotide”, “nucleotides”, “nucleic acids”, and “nucleic acid molecules” are used interchangeably unless otherwise specifically indicated and, similarly to the amino acids, are referred to by their commonly accepted single-letter codes. Similar to the amino acids, they encompass both naturally-occurring and non-naturally occurring nucleic acid polymers. The polynucleotide, oligonucleotide, nucleotides, nucleic acids, or nucleic acid molecules may be composed of DNA, RNA or a combination thereof.

In the context of the present invention, the phrase “control level” refers to a gene or protein expression level detected in a control sample and may include (a) a normal control level or (b) a cancer specific control level, more particularly a pancreatic cancer specific control level. A control level can be a single expression pattern from a single reference population or composed from a plurality of expression patterns. The phrase “normal control level” refers to a level of gene expression detected in a normal, healthy individual or in a population of individuals known not to be suffering from cancer, such pancreatic cancer, e.g., PDAC. A normal individual is one with no clinical symptoms of cancer, such pancreatic cancer, e.g., PDAC. On the other hand, a “pancreatic cancer (PC) control level” or a “PDAC control level” refers to a level of gene expression found in a population suffering from pancreatic cancer (PC) and pancreatic ductal adenocarcinoma (PDAC), respectively.

A similarity in C2orf18 expression levels between a test sample and a PC or PDAC control indicates that the subject (from which the test sample was obtained) suffers from or is at risk of developing PC or PDAC, respectively. According to the present invention, an expression level of a particular gene is deemed “increased” when expression of the gene is increased by at least 1.1, preferably more than 1.5, preferably more than 2.0, preferably more than 5.0, preferably more than 10.0 or more fold as compared to a control level. Likewise, an expression level of a particular gene is deemed “decreased” when expression of the gene is decreased by at least 1.1, preferably more than 1.5, preferably more than 2.0, preferably more than 5.0, preferably more than 10.0 or more fold as compared to a control level. C2orf18 gene expression can be determined by detecting mRNA of C2orf18 from a tissue sample from a subject, e.g., by RT-PCR or Northern blot analysis, or detecting a protein encoded by C2orf18, e.g., by immunohistochemical analysis of a tissue sample from a subject.

The C2orf 18 Gene and C2orf 18 Protein:

The present invention is based in part on the discovery that the gene encoding C2orf18 is over-expressed in PDAC as compared to non-cancerous tissue. C2orf18 is also referred to herein as PAMP (pancreas cancer mitochondrial protein). The present invention relates to the C2orf 18 gene and the C2orf 18 protein encoded thereby and the use thereof in the context of pancreatic cancer diagnostics and therapeutics. The cDNA identified for C2orf18 is 3912 nucleotides in length. The nucleic acid and polypeptide sequences of C2orf18 are shown in SEQ ID NO: 11 and 12, respectively. The sequence data are also available via following accession numbers.

C2ORF18/C2orf18/ANT2BP: NM_{—}017877.

In the context of the present invention, functional equivalents are also considered to be “C2orf 18 polypeptides”. Herein, a “functional equivalent” of a protein is a polypeptide that has a biological activity equivalent to the protein. Namely, any polypeptide that retains the biological ability of the C2orf18 protein may be used as such a functional equivalent in the present invention. In the context of the instant invention, it is preferable that the biological ability retained by the C2orf 18 functional equivalent is the cell proliferation promoting activity associated with the native C2orf 18 protein. The cell proliferating activity of a biological sample can be determined by detecting the speed of proliferation, or by measuring the cell cycle or the colony forming ability. Such activities may be routinely assayed using conventional technology and standard assays, such as the MTT Cell Proliferation Assay available through ATCC (Manassas, Va.) or the ViaLight™ Assay from Lonza (Base1 Switzerland). Also of interest is the ability of the native C2orf18 protein to bind ANT2.

Examples of functional equivalents include those wherein one or more amino acids are substituted, deleted, added, or inserted to the natural occurring amino acid sequence of the C2orf18 protein. Alternatively, the polypeptide may be composed an amino acid sequence having at least about 80% homology (also referred to as sequence identity) to the sequence of the respective protein, more preferably at least about 90% to 95% homology, even more preferably 99% homology. In other embodiments, the polypeptide can be encoded by a polynucleotide that hybridizes under stringent conditions to the natural occurring nucleotide sequence of the C2orf18 gene.

A polypeptide of the present invention may have variations in amino acid sequence, molecular weight, isoelectric point, the presence or absence of sugar chains, or form, depending on the cell or host used to produce it or the purification method utilized. Nevertheless, as long as it has a function equivalent to that of the human C2orf18 protein of the present invention, it is within the scope of the present invention.

The phrase “stringent (hybridization) conditions” refers to conditions under which a nucleic acid molecule will hybridize to its target sequence, typically in a complex mixture of nucleic acids, but not detectably to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times of background, preferably 10 times of background hybridization. Exemplary stringent hybridization conditions include the following: 50% formamide, 5×SSC, and 1% SDS, incubating at 42 degrees C., or, 5×SSC, 1% SDS, incubating at 65 degrees C., with wash in 0.2×SSC, and 0.1% SDS at 50 degrees C.

In the context of the present invention, a condition of hybridization for isolating a DNA encoding a polypeptide functionally equivalent to the human C2orf 18 protein can be routinely selected by a person skilled in the art. For example, hybridization may be performed by conducting pre-hybridization at 68° C. for 30 min or longer using “Rapid-hyb buffer” (Amersham LIFE SCIENCE), adding a labeled probe, and warming at 68° C. for 1 hour or longer. The following washing step can be conducted, for example, in a low stringent condition. An exemplary low stringent condition may include 42 degrees C., 2×SSC, 0.1% SDS, preferably 50 degrees C., 2×SSC, 0.1% SDS. High stringency conditions are often preferably used. An exemplary high stringency condition may include washing 3 times in 2×SSC, 0.01% SDS at room temperature for 20 min, then washing 3 times in 1×SSC, 0.1% SDS at 37 degrees C. for 20 min, and washing twice in 1×SSC, 0.1% SDS at 50 degrees C. for 20 min. However, several factors, such as temperature and salt concentration, can influence the stringency of hybridization and one skilled in the art can suitably select the factors to achieve the requisite stringency.

Generally, it is known that modifications of one or more amino acid in a protein do not influence the function of the protein. In fact, mutated or modified proteins, proteins having amino acid sequences modified by substituting, deleting, inserting, and/or adding one or more amino acid residues of a certain amino acid sequence, have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)). Accordingly, one of skill in the art will recognize that individual additions, deletions, insertions, or substitutions to an amino acid sequence which alter a single amino acid or a small percentage of amino acids or those considered to be a “conservative modifications”, wherein the alteration of a protein results in a protein with similar functions, are acceptable in the context of the instant invention.

So long as the activity the protein is maintained, the number of amino acid mutations is not particularly limited. However, it is generally preferred to alter 5% or less of the amino acid sequence. Accordingly, in a preferred embodiment, the number of amino acids to be mutated in such a mutant is generally 30 amino acids or less, preferably 20 amino acids or less, more preferably 10 amino acids or less, more preferably 6 amino acids or less, and even more preferably 3 amino acids or less.

An amino acid residue to be mutated is preferably mutated into a different amino acid in which the properties of the amino acid side-chain are conserved (a process known as conservative amino acid substitution). Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). Conservative substitution tables providing functionally similar amino acids are well known in the art. For example, the following eight groups each contain amino acids that are conservative substitutions for one another:

1) Alanine (A), Glycine (G);

2) Aspartic acid (D), Glutamic acid (E);

3) Aspargine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cystein (C), Methionine (M) (see, e.g., Creighton, Proteins 1984).

Such conservatively modified polypeptides are included in the context of the present invention and, so long as they retain the biological activity of the native C2orf18 protein, are considered to be “C2orf18 polypeptides”. However, the present invention is not restricted thereto and the C2orf18 polypeptide may include non-conservative modifications, so long as at least one biological activity of the C2orf18 protein (such as stimulation of cell proliferation) is retained. Furthermore, the modified proteins do not exclude polymorphic variants, interspecies homologues, and those encoded by alleles of these proteins.

Moreover, the C2orf18 gene of the present invention encompasses polynucleotides that encode such functional equivalents of the C2orf 18 protein. In addition to hybridization, a gene amplification method, for example, the polymerase chain reaction (PCR) method, can be utilized to isolate a polynucleotide encoding a polypeptide functionally equivalent to the C2orf18 protein, using a primer synthesized based on the sequence information of the protein encoding DNA (SEQ ID NO: 11). Polynucleotides and polypeptides that are functionally equivalent to the human C2orf 18 gene and protein, respectively, normally have a high homology to the originating nucleotide or amino acid sequence thereof. “High homology” typically refers to a homology of 40% or higher, preferably 60% or higher, more preferably 80% or higher, even more preferably 90% to 95% or higher. The homology of a particular polynucleotide or polypeptide can be determined by following the algorithm in “Wilbur and Lipman, Proc Natl Acad Sci USA 80: 726-30 (1983)”.

Antibodies:

The present invention also provides antibodies against C2orf18, or immunologically active fragments of such antibodies. Such antibodies find utility in the detection of C2orf18 specific expression. In the context of the present invention, immunohistochemical analyses using a polyclonal antibody specific to C2orf18 validated its overexpression in pancreatic cancer cells and no or very limited expression in normal adult vital organs (heart, lung, kidney, liver, and brain). Therefore, the antibodies of the present invention are useful for detecting C2orf18 protein in the biopsy from a subject, diagnosing C2orf18-related diseases, for example pancreatic cancer, e.g. PDAC, and treating those diseases. Furthermore, the antibodies of the present invention may be useful tools for functional analysis of C2orf18. An antibody of the present invention can be prepared by using C2orf18 fragments having an amino acid sequence set forth in CRAAGQSDSSVDPQQPF (SEQ ID NO: 5) and/or AEESEQERLLGGTRTPINDAS (SEQ ID NO: 6). Therefore antibodies recognize an epitope consisting of the amino acid sequence CRAAGQSDSSVDPQQPF (SEQ ID NO: 5) and/or AEESEQERLLGGTRTPINDAS (SEQ ID NO: 6) are included in the present invention. More specifically, in a preferred embodiment, an antibody of the present invention recognizes or binds to a polypeptide consisting the amino acid sequence of SEQ ID NO: 5 or SEQ ID NO: 6.

The term “antibody” as used herein encompasses naturally occurring antibodies as well as non-naturally occurring antibodies, including, for example, single chain antibodies, chimeric, bifunctional and humanized antibodies, as well as antigen-binding fragments thereof, (e.g., Fab′, F(ab′)₂, Fab, Fv and rIgG). See also, Pierce Catalog and Handbook, 1994-1995 (Pierce Chemical Co., Rockford, Ill.). See also, e.g. Kuby, J., Immunology, 3rd Ed., W.H. Freeman & Co., New York (1998). Such non-naturally occurring antibodies can be constructed using solid phase peptide synthesis, can be produced recombinantly or can be obtained, for example, by screening combinatorial libraries consisting of variable heavy chains and variable light chains as described by Huse et al., Science 246:1275-81 (1989), which is incorporated herein by reference. These and other methods of making, for example, chimeric, humanized, CDR-grafted, single chain, and bifunctional antibodies are well known to those skilled in the art (Winter and Harris, Immunol. Today 14:243-6 (1993); Ward et al., Nature 341:544-6 (1989); Harlow and Lane, Antibodies, 511-52, Cold Spring Harbor Laboratory publications, New York, 1988; Hilyard et al., Protein Engineering: A practical approach (IRL Press 1992); Borrebaeck, Antibody Engineering, 2d ed. (Oxford University Press 1995); each of which is incorporated herein by reference).

The term “antibody” includes both polyclonal and monoclonal antibodies. The term also includes genetically engineered forms such as chimeric antibodies (e.g., humanized murine antibodies) and heteroconjugate antibodies (e.g., bispecific antibodies). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Holliger et al. (1993) Proc Natl Acad Sci USA. 90:6444, Gruber et al. (1994) J Immunol:5368, Zhu et al. (1997) Protein Sci 6:781, Hu et al. (1997) Cancer Res. 56:3055, Adams et al. (1993) Cancer Res. 53:4026, and McCartney, et al. (1995) Protein Eng. 8:301.

Typically, an antibody has a heavy and light chain. Each heavy and light chain contains a constant region and a variable region, (the regions are also known as “domains”). Light and heavy chain variable regions contain four “framework” regions interrupted by three hyper-variable regions, also called “complementarity-determining regions” or “CDRs”. The extent of the framework regions and CDRs have been defined. The sequences of the framework regions of different light or heavy chains are relatively conserved within a species. The framework region of an antibody, that is the combined framework regions of the constituent light and heavy chains, serves to position and align the CDRs in three dimensional spaces.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found.

References to “VH” refer to the variable region of an immunoglobulin heavy chain of an antibody, including the heavy chain of an Fv, scFv, or Fab. References to “VL” refer to the variable region of an immunoglobulin light chain, including the light chain of an Fv, scFv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “humanized antibody” is an immunoglobulin molecule that contains minimal sequence derived from non-human immunoglobulin. Humanized antibodies include human immunoglobulins (recipient antibody) in which residues from a complementarity-determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also include residues which are found neither in the recipient antibody nor in the imported CDR or framework sequences. In general, a humanized antibody will include substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will include at least a portion of an immunoglobulin constant region (Fc), typically that of a human immunoglobulin (Jones et al., Nature 321:522-5 (1986); Riechmann et al., Nature 332:323-7 (1988); and Presta, Curr. Op. Struct. Biol. 2:593-6 (1992)). Humanization can be essentially performed following the method of Winter and co-workers (Jones et al., Nature 321:522-5 (1986); Riechmann et al., Nature 332:323-7 (1988); Verhoeyen et al., Science 239:1534-6 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

The terms “epitope”, “antigenic” and “determinant” refer to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, X-ray crystallography and 2-dimensional nuclear magnetic resonance. See, e.g., Epitope Mapping Protocols in Methods in Molecular Biology, Vol. 66, Glenn E. Morris, Ed (1996).

The terms “non-antibody binding protein” or “non-antibody ligand” or “antigen binding protein” interchangeably refer to antibody mimics that use non-immunoglobulin protein scaffolds, including adnectins, avimers, single chain polypeptide binding molecules, and antibody-like binding peptide mimetics, as discussed in more detail below.

Other compounds have been developed that target and bind to targets in a manner similar to antibodies. Certain of these “antibody mimics” use non-immunoglobulin protein scaffolds as alternative protein frameworks for the variable regions of antibodies.

For example, Ladner et al. (U.S. Pat. No. 5,260,203) describe single polypeptide chain binding molecules with binding specificity similar to that of the aggregated, but molecularly separate, light and heavy chain variable region of antibodies. The single-chain binding molecule contains the antigen binding sites of both the heavy and light variable regions of an antibody connected by a peptide linker and will fold into a structure similar to that of the two peptide antibody. The single-chain binding molecule displays several advantages over conventional antibodies, including, smaller size, greater stability and are more easily modified.

Ku et al. (Proc. Natl. Acad. Sci. USA 92(14):6552-6556 (1995)) discloses an alternative to antibodies based on cytochrome b562. Ku et al. (1995) generated a library in which two of the loops of cytochrome b562 were randomized and selected for binding against bovine serum albumin. The individual mutants were found to bind selectively with BSA similarly with anti-BSA antibodies.

Lipovsek et al. (U.S. Pat. Nos. 6,818,418 and 7,115,396) discloses an antibody mimic featuring a fibronectin or fibronectin-like protein scaffold and at least one variable loop. Known as Adnectins, these fibronectin-based antibody mimics exhibit many of the same characteristics of natural or engineered antibodies, including high affinity and specificity for any targeted ligand. Any technique for evolving new or improved binding proteins can be used with these antibody mimics.

The structure of these fibronectin-based antibody mimics is similar to the structure of the variable region of the IgG heavy chain. Therefore, these mimics display antigen binding properties similar in nature and affinity to those of native antibodies. Further, these fibronectin-based antibody mimics exhibit certain benefits over antibodies and antibody fragments. For example, these antibody mimics do not rely on disulfide bonds for native fold stability, and are, therefore, stable under conditions which would normally break down antibodies. In addition, since the structure of these fibronectin-based antibody mimics is similar to that of the IgG heavy chain, the process for loop randomization and shuffling can be employed in vitro that is similar to the process of affinity maturation of antibodies in vivo.

Beste et al. (Proc. Natl. Acad. Sci. USA 96(5):1898-1903 (1999)) discloses an antibody mimic based on a lipocalin scaffold (Anticalin™). Lipocalins are composed of a beta-barrel with four hypervariable loops at the terminus of the protein. Beste (1999), subjected the loops to random mutagenesis and selected for binding with, for example, fluorescein. Three variants exhibited specific binding with fluorescein, with one variant showing binding similar to that of an anti-fluorescein antibody. Further analysis revealed that all of the randomized positions are variable, indicating that Anticalin™ would be suitable to be used as an alternative to antibodies.

Anticalins™ are small, single chain peptides, typically between 160 and 180 residues, which provides several advantages over antibodies, including decreased cost of production, increased stability in storage and decreased immunological reaction.

Hamilton et al. (U.S. Pat. No. 5,770,380) discloses a synthetic antibody mimic using the rigid, non-peptide organic scaffold of calixarene, attached with multiple variable peptide loops used as binding sites. The peptide loops all project from the same side geometrically from the calixarene, with respect to each other. Because of this geometric confirmation, all of the loops are available for binding, increasing the binding affinity to a ligand. However, in comparison to other antibody mimics, the calixarene-based antibody mimic does not consist exclusively of a peptide, and therefore it is less vulnerable to attack by protease enzymes. Neither does the scaffold consist purely of a peptide, DNA nor RNA, meaning this antibody mimic is relatively stable in extreme environmental conditions and has a long life span. Further, since the calixarene-based antibody mimic is relatively small, it is less likely to produce an immunogenic response.

Murali et al. (Cell. Mol. Biol. 49(2):209-216 (2003)) discusses a methodology for reducing antibodies into smaller peptidomimetics, they term “antibody like binding peptidomemetics” (ABiP) which can also be useful as an alternative to antibodies.

Silverman et al. (Nat. Biotechnol. (2005), 23: 1556-1561) discloses fusion proteins that are single-chain polypeptides having multiple domains termed “avimers”. Developed from human extracellular receptor domains by in vitro exon shuffling and phage display, the avimers are a class of binding proteins somewhat similar to antibodies in their affinities and specificities for various target molecules. The resulting multidomain proteins can include multiple independent binding domains that can exhibit improved affinity (in some cases sub-nanomolar) and specificity compared with single-epitope binding proteins. Additional details concerning methods of construction and use of avimers are disclosed, for example, in U.S. Patent App. Pub. Nos. 20040175756, 20050048512, 20050053973, 20050089932 and 20050221384.

In addition to non-immunoglobulin protein frameworks, antibody properties have also been mimicked in compounds including RNA molecules and unnatural oligomers (e.g., protease inhibitors, benzodiazepines, purine derivatives and beta-turn mimics), all of which are suitable for use with the present invention.

Double Stranded Molecules:

The present invention also relates to the surprising discovery that inhibiting expression of C2orf18 is effective in inhibiting the cellular growth of cancer cells, including those involved in pancreatic cancer. The inventions described in this application are based in part on this discovery.

As used herein, the term “double-stranded molecule” refers to a nucleic acid molecule that inhibits expression of a target gene including, for example, short interfering RNA (siRNA; e.g., double-stranded ribonucleic acid (dsRNA) or small hairpin RNA (shRNA)) and short interfering DNA/RNA (siD/R-NA; e.g. double-stranded chimera of DNA and RNA (dsD/R-NA) or small hairpin chimera of DNA and RNA (shD/R-NA)). As used herein, the term “dsRNA” refers to a construct of two RNA molecules having complementary sequences to one another and that have annealed together via the complementary sequences to form a double-stranded RNA molecule. The nucleotide sequence of two strands may include not only the “sense” or “antisense” RNAs selected from a protein coding sequence of target gene sequence, but also RNA molecule having a nucleotide sequence selected from non-coding region of the target gene.

The term “shRNA”, as used herein, refers to an siRNA having a stem-loop structure, having a first and second regions complementary to one another, i.e., sense and antisense strands. The degree of complementarity and orientation of the first and second regions are sufficient such that base pairing occurs between the regions, the first and second regions are joined by a loop region, and the loop is resulted from a lack of base pairing between nucleotides (or nucleotide analogs) within the loop region. The loop region of an shRNA is a single-stranded region intervening between the sense and antisense strands and may also be referred to as “intervening single-strand”.

As used herein, the term “siD/R-NA” refers to a double-stranded polynucleotide molecule which is composed of both RNA and DNA, and includes hybrids and chimeras of RNA and DNA, and prevents translation of a target mRNA. Herein, a hybrid indicates a molecule wherein a polynucleotide composed of DNA and a polynucleotied composed of RNA hybridize to each other to form the double-stranded molecule; whereas a chimera indicates that one or both of the strands composing the double stranded molecule may contain RNA and DNA. Standard techniques of introducing siD/R-NA into the cell are used. The siD/R-NA includes a sense nucleic acid sequence (also referred to as “sense strand”), an antisense nucleic acid sequence (also referred to as “antisense strand”) or both. The siD/R-NA may be constructed such that a single transcript has both the sense and complementary antisense nucleic acid sequences from the target gene, e.g., a hairpin. The siD/R-NA may either be a dsD/R-NA or shD/R-NA.

As used herein, the term “dsD/R-NA” refers to a construct of two molecules having complementary sequences to one another and that have annealed together via the complementary sequences to form a double-stranded polynucleotide molecule. The nucleotide sequence of two strands may include not only the “sense” or “antisense” nucleotide sequence selected from a protein coding sequence of target gene sequence, but also polynucleotide having a nucleotide sequence selected from non-coding region of the target gene. One or both of the two molecules constructing the dsD/R-NA are composed of both RNA and DNA (chimeric molecule), or alternatively, one of the molecules is composed of RNA and the other is composed of DNA (hybrid double-strand).

The term “shD/R-NA”, as used herein, refers to an siD/R-NA having a stem-loop structure, having a first and second regions complementary to one another, i.e., sense and antisense strands. The degree of complementarity and orientation of the first and second regions are sufficient such that base pairing occurs between the regions, the first and second regions are joined by a loop region, and the loop is resulted from a lack of base pairing between nucleotides (or nucleotide analogs) within the loop region. The loop region of an shD/R-NA is a single-stranded region intervening between the sense and antisense strands and may also be referred to as “intervening single-strand”.

The double-stranded molecules of the invention may contain one or more modified nucleotides and/or non-phosphodiester linkages. Chemical modifications well known in the art are capable of increasing stability, availability, and/or cell uptake of the double-stranded molecule. The skilled person will be aware of other types of chemical modification which may be incorporated into the present molecules (WO03/070744; WO2005/045037). In one embodiment, modifications can be used to provide improved resistance to degradation or improved uptake. Examples of such modifications include phosphorothioate linkages, 2′-O-methyl ribonucleotides (especially on the sense strand of a double-stranded molecule), 2′-deoxy-fluoro ribonucleotides, 2′-deoxy ribonucleotides, “universal base” nucleotides, 5′-C-methyl nucleotides, and inverted deoxyabasic residue incorporation (US20060122137).

In another embodiment, modifications can be used to enhance the stability or to increase targeting efficiency of the double-stranded molecule. Modifications include chemical cross linking between the two complementary strands of a double-stranded molecule, chemical modification of a 3′ or 5′ terminus of a strand of a double-stranded molecule, sugar modifications, nucleobase modifications and/or backbone modifications, 2-fluoro modified ribonucleotides and 2′-deoxy ribonucleotides (WO2004/029212). In another embodiment, modifications can be used to increased or decreased affinity for the complementary nucleotides in the target mRNA and/or in the complementary double-stranded molecule strand (WO2005/044976). For example, an unmodified pyrimidine nucleotide can be substituted for a 2-thio, 5-alkynyl, 5-methyl, or 5-propynyl pyrimidine. Additionally, an unmodified purine can be substituted with a 7-deza, 7-alkyl, or 7-alkenyl purine. In another embodiment, when the double-stranded molecule is a double-stranded molecule with a 3′ overhang, the 3′-terminal nucleotide overhanging nucleotides may be replaced by deoxyribonucleotides (Elbashir S M et al., Genes Dev 2001 January 15, 15(2): 188-200). For further details, published documents such as US20060234970 are available. The present invention is not limited to these examples and any known chemical modifications may be employed for the double-stranded molecules of the present invention so long as the resulting molecule retains the ability to inhibit the expression of the target gene.

Furthermore, the double-stranded molecules of the present invention may include both DNA and RNA, e.g., dsD/R-NA or shD/R-NA. Specifically, a hybrid polynucleotide of a DNA strand and an RNA strand or a DNA-RNA chimera polynucleotide shows increased stability. Mixing of DNA and RNA, i.e., a hybrid type double-stranded molecule consisting of a DNA strand (polynucleotide) and an RNA strand (polynucleotide), a chimera type double-stranded molecule having both DNA and RNA on any or both of the single strands (polynucleotides), or the like may be formed for enhancing stability of the double-stranded molecule. The hybrid of a DNA strand and an RNA strand may be the hybrid in which either the sense strand is DNA and the antisense strand is RNA, or the opposite so long as it has an activity to inhibit expression of the target gene when introduced into a cell expressing the gene. Preferably, the sense strand polynucleotide is DNA and the antisense strand polynucleotide is RNA. Also, the chimera type double-stranded molecule may be either where both of the sense and antisense strands are composed of DNA and RNA, or where any one of the sense and antisense strands is composed of DNA and RNA so long as it has an activity to inhibit expression of the target gene when introduced into a cell expressing the gene.

In order to enhance stability of the double-stranded molecule, the molecule preferably contains as much DNA as possible, whereas to induce inhibition of the target gene expression, the molecule is required to be RNA within a range to induce sufficient inhibition of the expression. As a preferred example of the chimera type double-stranded molecule, an upstream partial region (i.e., a region flanking to the target sequence or complementary sequence thereof within the sense or antisense strands) of the double-stranded molecule is RNA. Preferably, the upstream partial region indicates the 5′ side (5′-end) of the sense strand and the 3′ side (3′-end) of the antisense strand. That is, in preferable embodiments, a region flanking to the 3′-end of the antisense strand, or both of a region flanking to the 5′-end of sense strand and a region flanking to the 3′-end of antisense strand consists of RNA. For instance, the chimera or hybrid type double-stranded molecule of the present invention include following combinations.

5′-[---DNA---]-3′
3′-(RNA)-[DNA]-5′:
antisense strand,
sense strand:
5′-(RNA)-[DNA]-3′
3′-(RNA)-[DNA]-5′:
antisense strand,
and
sense strand:
5′-(RNA)-[DNA]-3′
3′-(---RNA---)-5′:
antisense strand.

The upstream partial region preferably is a domain consisting of 9 to 13 nucleotides counted from the terminus of the target sequence or complementary sequence thereto within the sense or antisense strands of the double-stranded molecules. Moreover, preferred examples of such chimera type double-stranded molecules include those having a strand length of 19 to 21 nucleotides in which at least the upstream half region (5′ side region for the sense strand and 3′ side region for the antisense strand) of the polynucleotide is RNA and the other half is DNA. In such a chimera type double-stranded molecule, the effect to inhibit expression of the target gene is much higher when the entire antisense strand is RNA (US20050004064).

In the present invention, the double-stranded molecule may form a hairpin, such as a short hairpin RNA (shRNA) and short hairpin consisting of DNA and RNA (shD/R-NA). The shRNA or shD/R-NA is a sequence of RNA or mixture of RNA and DNA making a tight hairpin turn that can be used to silence gene expression via RNA interference. The shRNA or shD/R-NA includes the sense target sequence and the antisense target sequence on a single strand wherein the sequences are separated by a loop sequence. Generally, the hairpin structure is cleaved by the cellular machinery into dsRNA or dsD/R-NA, which is then bound to the RNA-induced silencing complex (RISC). This complex binds to and cleaves mRNAs which match the target sequence of the dsRNA or dsD/R-NA.

A double-stranded molecule against the C2orf18 gene (e.g. a “C2orf18 siRNA”) can be used to reduce the expression level of the gene. Herein, the term “siRNA” refers to a double-stranded RNA molecule which prevents translation of a target mRNA. In the context of the present invention, the double-stranded molecule is composed of a sense nucleic acid sequence and an anti-sense nucleic acid sequence against the C2orf18 gene. The double-stranded molecule is constructed so that it includes both a sense and complementary antisense sequences of the target sequence. The double-stranded molecule may either be a dsRNA, shRNA, ds D/RNA or shD/RNA.

A double-stranded molecule against the C2orf18 gene hybridizes to a target region of mRNA, i.e., associates with the normally single-stranded mRNA transcript at the region corresponding to the target sequence and thereby interfering with translation of the mRNA, which finally decreases or inhibits production (expression) of the polypeptide encoded by the gene. Thus, a double-stranded molecule of the invention can be defined by its ability to specifically hybridize to the mRNA of the C2orf 18 gene under stringent conditions.

In the context of the present invention, a double-stranded molecule is preferably less than 500, 200, 100, 50, or 25 nucleotides in length. More preferably a double-stranded molecule is 19-25 nucleotides in length. Exemplary target nucleic acid sequences of C2orf18 double-stranded molecule include the oligonucleotide sequence corresponding to a target sequence of C2orf18, e.g. 5′-GGAGCACAGCTTCCAGCAT-3′(SEQ ID NO: 7) or 5′-GCACGACAGTCAGCACAAG-3′(SEQ ID NO: 8). Accordingly, for example, the present invention provides double-stranded molecules having the oligonucleotide sequence comprising of SEQ ID NO: 7 or 8. At RNA region of the double stranded molecule the nucleotide “t” in the target sequence should be replaced with “u”. In order to enhance the inhibition activity of the double-stranded molecules, nucleotide “u” can be added to the 3′ end of the antisense strand. The number of “u”s to be added is at least 2, generally 2 to 10, preferably 2 to 5. The added “u”s form a single strand at the 3′ end of the antisense strand of the double-stranded molecule.

A loop sequence composed of an arbitrary nucleotide sequence can be located between the sense and antisense sequence in order to form the hairpin loop structure. Thus, the present invention also provides double-stranded molecule having the general formula 5′-[A]-[B]-[A′]-3′, wherein [A] is an oligonucleotide sequence corresponding to a sequence that specifically hybridizes to a target sequence of mRNA or a cDNA of the C2orf 18 gene. In preferred embodiments, [A] is an oligonucleotide sequence corresponding to a target sequence of the C2orf18 gene; [B] is a nucleotide sequence composed of 3 to 23 nucleotides; and [A′] is an oligonucleotide sequence composed of the complementary sequence of [A]. The region [A] hybridizes to [A′], and then a loop composed of region [B] is formed. The loop sequence may be preferably 3 to 23 nucleotide in length. The loop sequence, for example, can be selected from a group composed of following sequences (http://www.ambion.com/techlib/tb/tb_{—}506.html): CCC, CCACC, or CCACACC: Jacque J M et al., Nature 2002, 418: 435-8.

### UUCG: Lee N S et al., Nature Biotechnology 2002, 20:500-5; Fruscoloni P et al., Proc Natl Acad Sci USA 2003, 100(4):1639-44.

### UUCAAGAGA: Dykxhoom D M et al., Nature Reviews Molecular Cell Biology 2003, 4:457-67.

‘UUCAAGAGA (“ttcaagaga” in DNA)’ is a particularly suitable loop sequence.

Furthermore, loop sequence consisting of 23 nucleotides also provides an active siRNA (Jacque J-M et al., Nature 2002, 418:435-8).

Exemplary hairpin double-stranded molecule suitable for use in the context of the present invention include:

(for target sequence of SEQ ID NO: 7)
5′-GGAGCACAGCUUCCAGCAU-[B]-AUGCUGGAAGCUGUGCUCC-3′;
and
(for target sequence of SEQ ID NO: 8)
5′-GCACGACAGUCAGCACAAG-[B]-CUUGUGCUGACUGUCGUGC-3′.

Specifically, the following double-stranded molecules [1] to [13] are included in the present invention:

[1] A double-stranded molecule comprising a sense strand and an antisense strand, wherein the sense strand comprises a nucleotide sequence corresponding to a target sequence consisting of SEQ ID NO: 7 or 8, and wherein the antisense strand comprises a nucleotide sequence which is complementary to said sense strand, wherein said sense strand and said antisense strand hybridize to each other to form said double-stranded molecule, and wherein said double-stranded molecule, when introduced into a cell expressing the C2orf18 gene, inhibits expression of said gene.

[2] The double-stranded molecule of [1], wherein said target sequence comprises from about 19 to about 25 contiguous nucleotides from the nucleotide sequence consisting of SEQ ID NO: 11.

[3] The double-stranded molecule of [2], wherein said double-stranded molecule is a single nucleotide transcript comprising the sense strand and the antisense strand linked via a single-stranded nucleotide sequence.

[4] The double-stranded molecule of [3], which has a general formula 5′-[A]-[B]-[A′]-3′, wherein

[A] is the sense strand comprising an oligonucleotide corresponding to a sequence of SEQ ID NO: 7 or 8;
[B] is a nucleotide sequence consisting of about 3 to about 23 nucleotides; and[A′] is the antisense strand comprising an oligonucleotide corresponding to a sequence complementary to the sequence of [A].

[5] The double-stranded molecule of [3], which has a general formula 5′-[A]-[B]-[A′]-3′, wherein

[A] is a ribonucleotide sequence corresponding to a sequence consisting of SEQ ID NO: 7 or 8 as the target sequence;
[B] is a nucleotide sequence consisting of about 3 to about 23 nucleotides; and
[A′] is a ribonucleotide sequence consisting of the complementary sequence of [A].

[6] The double-stranded molecule of [1]-[5], which comprises RNA.

[7] The double-stranded molecule of [1]-[5], which comprises both DNA and RNA.

[8] The double-stranded molecule of [7], which is a hybrid of a DNA polynucleotide and an RNA polynucleotide.

[9] The double-stranded molecule of [8] wherein the sense and the antisense strands are made of DNA and RNA, respectively.

[10] The double-stranded molecule of [7], which is a chimera of DNA and RNA.

[11] The double-stranded molecule of [10], wherein a 5′-end region of the target sequence in the sense strand, and/or a 3′-end region of the complementary sequence of the target sequence in the antisense strand consists of RNA.

[12] The double-stranded molecule of [11], wherein the RNA region consists of 9 to 13 nucleotides.

[13] The double-stranded molecule of [1]-[5], which contains 3′ overhang.

The double-stranded molecule of the present invention will be described in more detail below.

The oligonucleotide sequence of suitable double-stranded molecules can be designed using a design computer program available from the Ambion website (http://www.ambion.com/techlib/misc/siRNA_finder.html). The computer program selects nucleotide sequences for double-stranded molecule synthesis based on the following protocol.

Selection of Target Sites:

1. Beginning with the AUG start codon of the object transcript, scan downstream for AA dinucleotide sequences. Record the occurrence of each AA and the 3′ adjacent 19 nucleotides as potential target sites. Tuschl et al. Genes Cev 1999, 13(24):3191-7 don't recommend against designing target sequence to the 5′ and 3′ untranslated regions (UTRs) and regions near the start codon (within 75 nucleotides) as these may be richer in regulatory protein binding sites. UTR-binding proteins and/or translation initiation complexes may interfere with binding of the endonuclease complex.

2. Compare the potential target sites to the human genome database and eliminate from consideration any target sequences with significant homology to other coding sequences. The homology search can be performed using BLAST (Altschul S F et al., Nucleic Acids Res 1997, 25:3389-402; J Mol Biol 1990, 215:403-10.), which can be found on the NCBI server at: www.ncbi.nlm.nih.gov/BLAST/.

3. Select qualifying target sequences for synthesis. At Ambion, preferably several target sequences can be selected along the length of the gene to evaluate.

Standard techniques for introducing a double-stranded molecule into the cell may be used. For example, a double-stranded molecule of C2orf18 can be directly introduced into the cells in a form that is capable of binding to the mRNA transcripts. In these embodiments, the double-stranded molecules of the present invention are typically modified as described above for antisense molecules. Other modifications are also possible, for example, cholesterol-conjugated double-stranded molecules have shown improved pharmacological properties (Song et al., Nature Med 2003, 9:347-51).

Alternatively, a DNA encoding the double-stranded molecule may be carried in a vector (hereinafter, also referred to as ‘siRNA vector’). Such vectors may be produced, for example, by cloning the target C2orf18 gene sequence into an expression vector having operatively-linked regulatory sequences (e.g., a RNA polymerase III transcription unit from the small nuclear RNA (snRNA) U6 or the human H1 RNA promoter) flanking the sequence in a manner that allows for expression (by transcription of the DNA molecule) of both strands (Lee N S et al., Nature Biotechnology 2002, 20: 500-5). For example, an RNA molecule that is antisense to mRNA of the C2orf18 gene is transcribed by a first promoter (e.g., a promoter sequence 3′ of the cloned DNA) and an RNA molecule that is the sense strand for the mRNA of the C2orf 18 gene is transcribed by a second promoter (e.g., a promoter sequence 5′ of the cloned DNA). The sense and antisense strands hybridize in vivo to generate double-stranded molecule constructs for silencing the expression of the C2orf18 gene. Alternatively, the two constructs can be utilized to create the sense and anti-sense strands of a single-stranded construct. In this case, a construct having secondary structure, e.g., hairpin, is produced as a single transcript that includes both the sense and complementary antisense sequences of the target gene.

Specifically, the present invention provides a vector having each or both of a combination of polynucleotide having a sense strand nucleic acid and an antisense strand nucleic acid, wherein said sense strand nucleic acid includes nucleotide sequence of SEQ ID NOs: 7 or 8, and wherein the antisense strand includes a nucleotide sequence which is complementary to said sense strand, wherein the transcripts of said sense strand and said antisense strand hybridize to each other to form said double-stranded molecule, and wherein said vector, when introduced into a cell expressing the C2orf18 gene, inhibits expression of said gene.

Alternatively, the present invention provides vectors having each of a combination of polynucleotide having a sense strand nucleic acid and an antisense strand nucleic acid, wherein said sense strand nucleic acid includes nucleotide sequence of SEQ ID NOs: 7 or 8, and said antisense strand nucleic acid includes a sequence complementary to the sense strand, wherein the transcripts of said sense strand and said antisense strand hybridize to each other to form a double-stranded molecule, and wherein said vectors, when introduced into a cell expressing the C2orf18 gene, inhibits expression of said gene. Preferably, the polynucleotide is an oligonucleotide of between about 19 and 25 nucleotides in length (e.g., contiguous nucleotides from the nucleotide sequence of SEQ ID NO: 11). More preferably, the combination of polynucleotide includes a single nucleotide transcript having the sense strand and the antisense strand linked via a single-stranded nucleotide sequence. More preferably, the combination of polynucleotide has the general formula 5′-[A]-[B]-[A]-3′, wherein [A] is a nucleotide sequence having SEQ ID NO: 7 or 8; [B] is a nucleotide sequence consisting of about 3 to about 23 nucleotide; and [A′] includes a nucleotide sequence complementary to [A].

For introducing the vector of double-stranded molecule into the cell, transfection-enhancing agent can be used. FuGENE6 (Roche diagnostics), Lipofectamine 2000 (Invitrogen), Oligofectamine (Invitrogen), and Nucleofector (Wako pure Chemical) are useful as the transfection-enhancing agent.

Methods of Detecting or Diagnosing Pancreatic Cancer:

Among dozens of genes trans-activated in pancreatic cancer cells, the present invention focuses on one novel gene C2orf18 (GenBank™ Accession No. NM_{—}017877), which encodes a multiple trans-membrane protein. It is referred to herein interchangeably as “ANT2BP” (ANT2-binding protein) and “PAMP” (pancreas cancer mitochondrial protein). RT-PCR, northern-blot, and immunohistochemical analysis using polyclonal antibody specific to C2orf18 validated its over-expression in pancreatic cancer cells and no or very limited expression in normal adult vital organs (heart, lung, kidney, liver, and brain).

Accordingly, the present invention features a method of detecting, diagnosing and/or determining the presence of or disposition for developing cancer, e.g. pancreatic cancer in a subject by determining an expression level of C2orf18 in a subject derived biological sample, such as tissue sample. An alteration, e.g., increase of the level of expression of C2orf18 as compared to a normal control level, indicates that the subject suffers from or is at risk of developing cancer, e.g. pancreatic cancer.

The present invention involves determining (e.g., measuring) the expression level of a C2orf18 gene. Using sequence information provided by the GenBank™ database entries for known sequences, the C2orf18 gene can be detected and measured using techniques well known to one of ordinary skill in the art. For example, sequences within the sequence database entries corresponding to C2orf18 gene, can be used to construct probes for detecting RNA sequences corresponding to C2orf18 gene in, e.g., Northern blot hybridization analyses. Probes typically include at least 10, at least 20, at least 50, at least 100, or at least 200 nucleotides of a C2orf18 sequence. As another example, the sequences can be used to construct primers for specifically amplifying the C2orf18 nucleic acid in, e.g., amplification-based detection methods, for example, reverse-transcription based polymerase chain reaction. As another example, the antibody against C2orf18, e.g., anti-C2orf18 polyclonal antibody or anti-C2orf18 monoclonal antibody, can be used for immunoassay, for example, immunohistochemical analysis, western blot analysis or ELISA, etc.

Furthermore, the transcription product of the C2orf18 gene may be quantified using primers by amplification-based detection methods (e.g., RT-PCR). Such primers can also be prepared based on the available sequence information of the gene. For example, the primers (SEQ ID NOs: 3 and 4) used in the Example may be employed for the detection by RT-PCR, but the present invention is not restricted thereto.

Alternatively, the translation product may be detected for the diagnosis of the present invention. For example, the quantity of the C2orf 18 protein may be determined. A method for determining the quantity of the protein as the translation product includes immunoassay methods that use an antibody or antibody mimic specifically recognizing the protein. The antibody may be monoclonal or polyclonal. Furthermore, any fragment or modification (e.g., chimeric antibody, scFv, Fab, F(ab′)₂, Fv, etc.) of the antibody may be used for the detection, so long as the fragment retains the binding ability to the C2orf18 protein. Methods to prepare these kinds of antibodies for the detection of proteins are well known in the art, and any method may be employed in the present invention to prepare such antibodies and equivalents thereof.

As another method to detect the expression level of the C2orf18 gene based on its translation product, the intensity of staining may be observed via immunohistochemical analysis using an antibody or antibody mimic against the C2orf18 protein. Namely, the observation of strong staining indicates increased presence of the protein and at the same time high expression level of the C2orf18 gene.

Furthermore, the translation product may be detected based on its biological activity. Specifically, herein, the C2orf 18 protein was demonstrated to be involved in the proliferation of cancer cells. Thus, the cell proliferative activity of the C2orf18 protein may be used as an index of the C2orf18 protein existing in the biological sample.

Expression level of C2orf18 gene in a test cell population, e.g., a tissue sample from a subject, is then compared to the expression level(s) of the gene in a reference cell population. The reference cell population includes one or more cells for which the compared parameter is known, e.g., pancreatic cancer cells, normal pancreatic cells or normal pancreatic ductal epithelial cells.

Whether or not an expression level of gene in a test cell population as compared to a reference cell population indicates cancer, e.g. pancreatic cancer, or a predisposition thereto depends upon the composition of the reference cell population. For example, if the reference cell population is composed of normal cells (not cancer), a similarity in gene expression level between the test cell population and the reference cell population indicates the test cell population is not cancer or not at the risk of it. Conversely, if the reference cell population is made up of cancer cells, a similarity in gene expression between the test cell population and the reference cell population indicates that the test cell population includes cancer cells.

A level of expression of a C2orf18 gene in a test cell population is considered “altered” or “differ” if it varies from the expression level of the C2orf18 gene in a reference cell population by more than 1.1, more than 1.5, more than 2.0, more than 5.0, more than 10.0 or more fold.

Differential gene expression between a test cell population and a reference cell population can be normalized to a control nucleic acid, e.g. a housekeeping gene. For example, a control nucleic acid is one which is known not to differ depending on the cancerous or non-cancerous state of the cell. The expression level of a control nucleic acid can be used to normalize signal levels in the test and reference cell populations. Exemplary control genes include, but are not limited to, e.g., beta-actin, glyceraldehyde 3 phosphate dehydrogenase and ribosomal protein P1.

The test cell population can be compared to multiple reference cell populations. Each of the multiple reference cell populations can differ in the known parameter. Thus, a test cell population can be compared to a first reference cell population known to contain, e.g., pancreatic cancer cells, as well as a second reference cell population known to be normal cells, e.g. not contain pancreatic cancer cells. The test cell population can include a tissue or cell sample from a subject known to contain, or suspected of containing, cancer cells.

The test cell population can be obtained from biopsy, e.g. a bodily tissue or a bodily fluid, e.g., biological fluid (for example, blood, sputum, saliva). For example, the test cell population can be purified from pancreatic tissue from subject suspected suffering from a cancer, e.g. pancreatic cancer. Preferably, the test cell population includes an epithelial cell. The epithelial cell is preferably from a tissue known to be or suspected to be a cancer, e.g. pancreatic cancer.

Cells in the reference cell population are preferably from a tissue type similar to that of the test cell population. Optionally, the reference cell population is a cell line, e.g. a pancreatic cancer cell line or PDAC cell line (i.e., a positive control) or a normal non-cancerous cell line (i.e., a negative control). Alternatively, the control cell population can be derived from a database of molecular information from cells for which the assayed parameter or condition is known.

The subject to be diagnosed is preferably a mammal. Exemplary mammals include, but are not limited to, a human, non-human primate, mouse, rat, dog, cat, horse, or cow.

Alternatively, according to the present invention, an intermediate result for examining the condition of a subject may be provided. Such intermediate result may be combined with additional information to assist a doctor, nurse, or other practitioner to determine that a subject suffers from cancer, e.g. pancreatic cancer.

Alternatively, the present invention may be used to detect cancerous cells in a subject-derived tissue, and provide a doctor with useful information to determine that the subject suffers from cancer, e.g. pancreatic cancer. Accordingly, the present invention involves determining (e.g., measuring) the level of C2orf18 in subject derived samples, such as pancreatic tissue samples. In the present invention, a method for diagnosing cancer, e.g. pancreatic cancer, also includes a method for testing or detecting cancer, e.g. pancreatic cancer. Alternatively, in the present invention, diagnosing cancer also refers to showing a suspicion, risk, or possibility of cancer in a subject.

Monitoring and Assessing the Efficacy of a Cancer Treatment:

The C2orf18 gene is differentially expressed between normal and cancerous cells and therefore allows for the course of cancer treatment to be monitored, wherein the above-described method for diagnosing cancer can be adapted and applied for monitoring and assessing the efficacy of a treatment on cancer, e.g. pancreatic cancer. Specifically, the efficacy of a treatment on cancer can be assessed by determining the expression level of the C2orf18 gene in a cell(s) derived from a subject undergoing the treatment. If desired, test cell populations are obtained from the subject at various time points, before, during, and/or after the treatment. The expression level of the C2orf18 gene can be, for example, determined following the method described above. In the context of the present invention, it is preferable to use the C2orf18 gene expression in a cell(s) not exposed to the treatment of interest as the control level to which the detected expression level is compared

If the expression level of the C2orf18 gene is compared to a control level that is determined from a normal cell or a cell population containing non-cancerous cell, e.g. non-pancreatic cancer cells, a similarity in the expression level indicates that the treatment of interest is efficacious and a difference in the expression level indicates less favorable clinical outcome or prognosis of that treatment. On the other hand, if the comparison is conducted against a control level that is determined from a cancer cell or a cell population containing cancer cells, e.g. pancreatic cancer cells, a difference in the expression level indicates efficacious treatment, while a similarity in the expression level indicates less favorable clinical outcome or prognosis.

Furthermore, the expression levels of the C2orf18 gene before and after a treatment can be compared according to the present method to assess the efficacy of the treatment. Specifically, the expression level detected in a subject-derived biological sample after a treatment (i.e., post-treatment level) is compared to the expression level detected in a biological sample obtained prior to the treatment onset from the same subject (i.e., pre-treatment level). A decrease in the post-treatment level compared to the pre-treatment level indicates that the treatment of interest is efficacious while an increase in or similarity of the post-treatment level to the pre-treatment level indicates less favorable clinical outcome or prognosis.

As used herein, the term “efficacious” indicates that the treatment leads to a reduction in the expression of a pathologically up-regulated gene, an increase in the expression of a pathologically down-regulated gene or a decrease in size, prevalence, or metastatic potential of carcinoma in a subject. When a treatment of interest is applied prophylactically, “efficacious” means that the treatment retards or prevents the forming of tumor or retards, prevents, or alleviates at least one clinical symptom of the disease. Assessment of the state of tumor in a subject can be made using standard clinical protocols.

In addition, efficaciousness of a treatment can be determined in association with any known method for diagnosing cancer. Cancers can be diagnosed, for example, by identifying symptomatic anomalies, e.g., weight loss, abdominal pain, back pain, anorexia, nausea, vomiting and generalized malaise, weakness, and jaundice.

Kits And Reagents for Detecting. Diagnosing or Determining Pancreatic Cancer:

The present invention provides a kit for detecting, diagnosing or determining cancer. Preferably, the cancer is pancreatic cancer, more preferably PDAC. Specifically, the kit includes at least one reagent for detecting the expression level of the C2orf18 in a subject-derived biological sample, which reagent may be selected from the group of:

(a) a reagent for detecting mRNA of the C2orf18;

(b) a reagent for detecting the C2orf 18 protein; and

(c) a reagent for detecting the biological activity of the C2orf 18.

Suitable reagents for detecting mRNA of the C2orf18 include nucleic acids that specifically bind to or identify the C2orf18 mRNA, such as oligonucleotides which have a complementary sequence to a part of the C2orf 18 mRNA. These kinds of oligonucleotides are exemplified by primers and probes that are specific to the C2orf 18 mRNA. These kinds of oligonucleotides may be prepared based on methods well known in the art. If needed, the reagent for detecting the C2orf 18 mRNA may be immobilized on a solid matrix.

On the other hand, suitable reagents for detecting the C2orf18 protein include an antibody to the C2orf18 protein. The antibody may be monoclonal or polyclonal. Furthermore, any fragment or modification (e.g., chimeric antibody, scFv, Fab, F(ab′)2, Fv, etc.) of the antibody may be used as the reagent, so long as the fragment retains the binding ability to the C2orf18 protein. Methods to prepare these kinds of antibodies for the detection of the protein are well known in the art, and any method may be employed in the present invention to prepare such antibodies and equivalents thereof. Furthermore, the antibody may be labeled with signal generating molecules via direct linkage or an indirect labeling technique. Labels and methods for labeling antibodies and detecting the binding of antibodies to their targets are well known in the art and any labels and methods may be employed for the present invention.

Furthermore, the biological activity can be determined by, for example, measuring the cell proliferating activity due to the expressed C2orf18 protein in the biological sample. For example, the cell is cultured in the presence of a subject-derived biological sample, and then by detecting the speed of proliferation, or by measuring the cell cycle or the colony forming ability, the cell proliferating activity of the biological sample can be determined.

Furthermore, the kit may include a solid matrix and reagent for binding a probe against the C2orf18 gene or antibody against the C2orf18 protein, a medium and container for culturing cells, positive and negative control reagents, and a secondary antibody for detecting an antibody against the C2orf18 protein. For example, tissue samples obtained from subjects with good prognosis or poor prognosis may serve as useful control reagents. A kit of the present invention may further include other materials desirable from a commercial and user standpoint, including buffers, diluents, filters, needles, syringes, and package inserts (e.g., written, tape, CD-ROM, etc.) with instructions for use. These reagents and such may be retained in a container with a label. Suitable containers include bottles, vials, and test tubes. The containers may be formed from a variety of materials, such as glass or plastic.

As an embodiment of the present invention, when the reagent is a probe against the C2orf 18 mRNA, the reagent may be immobilized on a solid matrix, such as a porous strip, to form at least one detection site. The measurement or detection region of the porous strip may include a plurality of sites, each containing a nucleic acid (probe). A test strip may also contain sites for negative and/or positive controls. Alternatively, control sites may be located on a strip separated from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of C2orf18 mRNA present in the sample. The detection sites may be configured in any suitably detectable shape and are typically in the shape of a bar or dot spanning the width of a test strip.

Alternatively, the present invention provides the above reagents for detecting, diagnosing or determining the presence or predisposition for developing pancreatic cancer.

Screening Methods:

Knockdown of endogenous C2orf18 by siRNA in cell over-expressing C2orf18 resulted in drastic suppression of the cell growth, suggesting its essential role in maintaining viability of the cells. Furthermore, immunocytochemical analysis and cell fractionation followed by western blot analysis suggested that C2orf18 was localized in the mitochondria, indicating that C2orf 18 might be involved with apoptosis or energy homeostasis in the cells. These findings on C2orf18 function and sub-cellular localization implicated that C2orf 18 could be a promising molecular target for pancreatic cancer therapy.

Therefore, the present invention provides a method of screening a candidate agent or compound for inhibiting proliferation of cell over-expressing C2orf18. The cell may be cancer cell, specifically pancreatic cancer cell, more specifically PDAC cell. Using the C2orf 18 gene, polypeptide encoded by the gene or fragments thereof, or transcriptional regulatory region of the gene, it is possible to screen for agents or compounds that inhibit the expression of the gene or the biological activity of a polypeptide encoded by the gene. In the context of the preset invention, the biological activity may be cell proliferative activity. Such agents or compounds can be a candidate agent or compound for pharmaceuticals for treating or preventing C2orf18-associated disease such as cancer, e.g. pancreatic cancer. Thus, the present invention further provides methods of identifying a candidate agent or compound for treating or preventing C2orf18-associated disease, such as cancer, specifically pancreatic cancer, more specifically PDAC, using the C2orf 18 gene, polypeptide encoded by the gene or fragments thereof, or transcriptional regulatory region of the gene.

An agent or compound identified by the screening method of the present invention is an agent or compound that inhibits the expression of the C2orf18 gene or the activity of the translation product of the gene and an agent or compound that expects to effective for treating C2orf18-associating disease, such as cancer, specifically pancreatic cancer, more specifically PDAC. Namely, the agents or compound identified through the present methods are expected to have a clinical benefit and can be further tested for an ability to prevent a growth of a cell over-expressing C2orf18 in animal models or test subjects.

In the context of the present invention, agents or compound to be identified through the present screening methods may be any biologics, any compound or composition including several compounds. Furthermore, the test agent or compound exposed to a cell or protein according to the screening methods of the present invention may be a single compound or a combination of compounds. When a combination of compounds is used in the methods, the compounds may be contacted sequentially or simultaneously.

Any test agents or compounds, for example, cell extracts, cell culture supernatants, products of fermenting microorganisms, extracts from marine organisms, plant extracts, purified or crude proteins, peptides, non-peptide compounds, synthetic micro-molecular compounds (including nucleic acid constructs, such as antisense RNAs, double-stranded molecules, siRNAs, ribozymes, etc.) and natural compounds can be used in the screening methods of the present invention. The test agent or compound of the present invention can be also obtained using any of the numerous approaches in combinatorial library methods known in the art, including, but not limited to,

(1) biological libraries,

(2) spatially addressable parallel solid phase or solution phase libraries,

(3) synthetic library methods requiring deconvolution,

(4) the “one-bead one-compound” library method and

(5) synthetic library methods using affinity chromatography selection.

The biological library approach is limited to peptide libraries, while the other four approaches are applicable to peptide, non-peptide oligomer or small molecule libraries of compounds (Lam, Anticancer Drug Des 1997, 12: 145-67). Examples of methods for the synthesis of molecular libraries can be found in the art (DeWitt et al., Proc Natl Acad Sci USA 1993, 90: 6909-13; Erb et al., Proc Natl Acad Sci USA 1994, 91: 11422-6; Zuckermann et al., J Med Chem 37: 2678-85, 1994; Cho et al., Science 1993, 261: 1303-5; Carell et al., Angew Chem Int Ed Engl 1994, 33: 2059; Carell et al., Angew Chem Int Ed Engl 1994, 33: 2061; Gallop et al., J Med Chem 1994, 37: 1233-51). Libraries of compounds may be presented in solution (see Houghten, Bio/Techniques 1992, 13: 412-21) or on beads (Lam, Nature 1991, 354: 82-4), chips (Fodor, Nature 1993, 364: 555-6), bacteria (U.S. Pat. No. 5,223,409), spores (U.S. Pat. Nos. 5,571,698; 5,403,484, and 5,223,409), plasmids (Cull et al., Proc Natl Acad Sci USA 1992, 89: 1865-9) or phage (Scott and Smith, Science 1990, 249: 386-90; Devlin, Science 1990, 249: 404-6; Cwirla et al., Proc Natl Acad Sci USA 1990, 87: 6378-82; Felici, J Mol Biol 1991, 222: 301-10; US Pat. Application 2002103360).

A compound in which a part of the structure of the compound identified by any of the present screening methods is converted by addition, deletion and/or replacement, is included in the agents or compound obtained by the screening methods of the present invention.

Furthermore, when the screened test agent or compound is a protein, or a DNA encoding the protein, either the whole amino acid sequence of the protein may be determined to deduce the nucleic acid sequence coding for the protein, or partial amino acid sequence of the obtained protein may be analyzed to prepare an oligo DNA as a probe based on the sequence, and screen cDNA libraries with the probe to obtain a DNA encoding the protein. The obtained DNA finds use in preparing the test agent or compound which is a candidate for treating or preventing cancer.

I. In Silico Screening Methods

Construction of test compound libraries is facilitated by knowledge of the molecular structure of compounds known to have the properties sought, and/or the molecular structure of the target molecules to be inhibited, i.e., C2orf18. One approach to preliminary screening of test compounds suitable for further evaluation is computer modeling of the interaction between the test compound and its target. In the present invention, modeling the interaction between the test compound and C2orf18 provides insight into the details of the interaction itself, and suggests possible strategies for disrupting the interaction, including potential molecular inhibitors of the interaction.

Computer modeling technology allows the visualization of the three-dimensional atomic structure of a selected molecule and the rational design of new compounds that will interact with the molecule. The three-dimensional construct typically depends on data from x-ray crystallographic analysis or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

An example of the molecular modeling system described generally above consists of the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al. Acta Pharmaceutica Fennica 97, 159-166 (1988); Ripka, New Scientist 54-57 (Jun. 16, 1988); McKinlay and Rossmann, Annu. Rev. Pharmacol. Toxiciol. 29, 111-122 (1989); Perry and Davies, Prog Clin Biol Res.291:189-93 (1989); Lewis and Dean, Proc. R. Soc. Lond B Biol Sci. 236, 125-40 and 141-62 (1989); and, with respect to a model receptor for nucleic acid components, Askew, et al., J. Am. Chem. Soc. 111, 1082-90 (1989).

Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. See, e.g., DesJarlais et al. (1988) J. Med. Chem. 31:722-9; Meng et al. (1992) J. Computer Chem. 13:505-24; Meng et al. (1993) Proteins 17:266-78; Shoichet et al. (1993) Science 259:1445-50.

Once a putative inhibitor of C2orf 18 has been identified, combinatorial chemistry techniques can be employed to construct any number of variants based on the chemical structure of the identified putative inhibitor. The resulting library of putative inhibitors, or “test agents” or “test compound” may be screened using the methods of the present invention to identify the test agent or compound that inhibit a biological activity of C2orf 18.

II. Protein Based Screening Methods

According to the present invention, the expression of the C2orf18 gene was suggested to be crucial for the growth and/or survival of cells over-expressing the gene, such as cancer cells, specifically pancreatic cancer cells, more specifically PDAC cells. Therefore, it was considered that agents or compounds which suppress the expression or function of the polypeptide encoded by the gene inhibit the growth and/or survival of the cells, and find use in inhibiting cell growth and treating or preventing cancer. Thus, the present invention provides methods of identifying a candidate agent or compound for inhibiting cell growth, or a candidate agent or compound for treating or preventing C2orf18-associating disease, using the C2orf18 polypeptide. In the present invention, the cell to be inhibited the growth is characterized by over-expression of C2orf18, such as cancer cell, e.g. pancreatic cancer cell, specifically pancreatic ductal adenocarcinoma cell. The C2orf18 associating disease is characterized by over-expression of C2orf18, such as cancer, e.g. pancreatic cancer, specifically PDAC.

In addition to the C2orf18 polypeptide, fragments of the polypeptide may be used in the context of the present screening methods, so long as at least one biological activity of natural occurring C2orf 18 polypeptide is retained.

The polypeptide or fragments thereof may be further linked to other substances so long as the resulting polypeptide and fragments retain at least one biological activity of the originating peptide. Usable substances include: peptides, lipids, sugar and sugar chains, acetyl groups, natural and synthetic polymers, etc. These kinds of modifications may be performed to confer additional functions or to stabilize the polypeptide and fragments.

The polypeptide or fragments used for the present method may be obtained from nature as naturally occurring proteins via conventional purification methods or through chemical synthesis based on the selected amino acid sequence. For example, conventional peptide synthesis methods that can be adopted for the synthesis include:

1) Peptide Synthesis, Interscience, New York, 1966;

2) The Proteins, Vol. 2, Academic Press, New York, 1976;

3) Peptide Synthesis (in Japanese), Maruzen Co., 1975;

4) Basics and Experiment of Peptide Synthesis (in Japanese), Maruzen Co., 1985;

5) Development of Pharmaceuticals (second volume) (in Japanese), Vol. 14 (peptide synthesis), Hirokawa, 1991;

6) WO99/67288; and

7) Barany G. & Merrifield R.B., Peptides Vol. 2, “Solid Phase Peptide Synthesis”, Academic Press, New York, 1980, 100-118.

Alternatively, the protein may be obtained adopting any known genetic engineering methods for producing polypeptides (e.g., Morrison J., J Bacteriology 1977, 132: 349-51; Clark-Curtiss & Curtiss, Methods in Enzymology (eds. Wu et al.) 1983, 101: 347-62). For example, first, a suitable vector including a polynucleotide encoding the objective protein in an expressible form (e.g., downstream of a regulatory sequence including a promoter) is prepared, transformed into a suitable host cell, and then the host cell is cultured to produce the protein. More specifically, a gene encoding the C2orf18 polypeptide is expressed in host (e.g., animal) cells and such by inserting the gene into a vector for expressing foreign genes, such as pSV2neo, pcDNA I, pcDNA3.1, pCAGGS, or pCD8. A promoter may be used for the expression. Any commonly used promoters may be employed including, for example, the SV40 early promoter (Rigby in Williamson (ed.), Genetic engineering, vol. 3. Academic Press, London, 1982, 83-141), the EF-alpha promoter (Kim et al., Gene 1990, 91:217-23), the CAG promoter (Niwa et al., Gene 1991, 108:193), the RSV LTR promoter (Cullen, Methods in Enzymology 1987, 152:684-704), the SRalpha promoter (Takebe et al., Mol Cell Biol 1988, 8:466), the CMV immediate early promoter (Seed et al., Proc Natl Acad Sci USA 1987, 84:3365-9), the SV40 late promoter (Gheysen et al., J Mol Appl Genet. 1982, 1:385-94), the Adenovirus late promoter (Kaufman et al., Mol Cell Biol 1989, 9:946), the HSV TK promoter, and such. The introduction of the vector into host cells to express the C2orf18 gene can be performed according to any methods, for example, the electroporation method (Chu et al., Nucleic Acids Res 1987, 15:1311-26), the calcium phosphate method (Chen et al., Mol Cell Biol 1987, 7:2745-52), the DEAE dextran method (Lopata et al., Nucleic Acids Res 1984, 12:5707-17; Sussman et al., Mol Cell Biol 1984, 4:1641-3), the Lipofectin method (Derijard B, Cell 1994, 7:1025-37); Lamb et al., Nature Genetics 1993, 5:22-30; Rabindran et al., Science 1993, 259:230-4), and such.

The C2orf 18 protein may also be produced in vitro adopting an in vitro translation system.

The C2orf18 polypeptide to be contacted with a test agent or compound can be, for example, a purified polypeptide, a soluble protein, or a fusion protein fused with other polypeptides.

II-1. Identifying Agents or Compounds that Bind to C2orf 18 Polypeptide

An agent or compound that binds to a protein is likely to alter the expression of the gene coding for the protein or the biological activity of the protein. Thus, in one aspect, the present invention provides a method of screening for an agent or compound for inhibiting cell growth and treating or preventing C2orf18 associating disease, which includes the steps of:

a) contacting a test agent or compound with the C2orf18 polypeptide or a functional fragment thereof;

b) detecting the binding between the polypeptide (or fragment) and the test agent or compound; and

c) selecting the test agent or compound that binds to the polypeptide (or fragment).

According to the present invention, the therapeutic effect of the test agent or compound on inhibiting cell growth and treating or preventing C2orf18 associating disease may be evaluated. Therefore, the present invention also provides a method of screening for an agent or compound for inhibiting cell growth and treating or preventing C2orf18 associating disease, which includes the steps of:

a) contacting a test agent or compound with the C2orf18 polypeptide or a functional fragment thereof;

b) detecting the binding between the polypeptide (or fragment) and the test agent or compound; and

c) correlating the binding of b) with the therapeutic effect of the test agent or compound.

In the present invention, the therapeutic effect may be correlated with the binding properties of the test agent or compound For example, when the test agent or compound binds to the polypeptide (or fragment), the test agent or compound may identified or selected as the candidate agent or compound having the therapeutic effect. Alternatively, when the test agent or compound does not bind to the polypeptide (or fragment), the test agent or compound may identified as the agent or compound having no significant therapeutic effect. The binding of a test agent or compound to the C2orf 18 polypeptide may be, for example, detected by immunoprecipitation using an antibody against the polypeptide. Therefore, for the purpose for such detection, it is preferred that the C2orf18 polypeptide or functional fragments thereof used for the screening contains an antibody recognition site. The antibody used for the screening may be one that recognizes an antigenic region (e.g., epitope) of the present C2orf18 polypeptide which preparation methods are well known in the art, and any method may be employed in the present invention to prepare such antibodies and equivalents thereof.

Alternatively, the C2orf18 polypeptide or functional fragments thereof may be expressed as a fusion protein including at its N- or C-terminus a recognition site (epitope) of a monoclonal antibody, whose specificity has been revealed, to the N- or C-terminus of the polypeptide. A commercially available epitope-antibody system can be used (Experimental Medicine 1995, 13:85-90). Vectors which can express a fusion protein with, for example, beta-galactosidase, maltose binding protein, glutathione S-transferase, green florescence protein (GFP), and such by the use of its multiple cloning sites are commercially available and can be used for the present invention. Furthermore, fusion proteins containing much smaller epitopes to be detected by immunoprecipitation with an antibody against the epitopes are also known in the art (Experimental Medicine 1995, 13:85-90). Such epitopes consisting of several dozen amino acids so as not to change the property of the C2orf 18 polypeptide or fragments thereof can also be used in the present invention. Examples include, but are not limited to, polyhistidine (His-tag), influenza aggregate HA, human c-myc, FLAG, Vesicular stomatitis virus glycoprotein (VSV-GP), T7 gene 10 protein (T7-tag), human simple herpes virus glycoprotein (HSV-tag), E-tag (an epitope on monoclonal phage), and such.

Glutathione S-transferase (GST) is also well-known as the counterpart of the fusion protein to be detected by immunoprecipitation. When GST is used as the protein to be fused with the C2orf18 polypeptide or fragment thereof to form a fusion protein, the fusion protein can be detected either with an antibody against GST or a substance specifically binding to GST, i.e., such as glutathione (e.g., glutathione-Sepharose 4B).

In immunoprecipitation, an immune complex is formed by adding an antibody (recognizing the C2orf18 polypeptide or a functional fragment thereof itself, or an epitope tagged to the polypeptide or fragment) to the reaction mixture of the C2orf18 polypeptide and the test agent or compound. If the test agent or compound has the ability to bind the polypeptide, then the formed immune complex will be composed of the C2orf18 polypeptide, the test agent or compound, and the antibody. On the contrary, if the test agent or compound is devoid of such ability, then the formed immune complex only include the C2orf 18 polypeptide and the antibody. Therefore, the binding ability of a test agent or compound to the C2orf18 polypeptide can be examined by, for example, measuring the size of the formed immune complex. Any method for detecting the size of a substance can be used, including chromatography, electrophoresis, and such. For example, when mouse IgG antibody is used for the detection, Protein A or Protein G sepharose can be used for quantitating the formed immune complex.

For more details on immunoprecipitation, see, for example, Harlow et al., Antibodies, Cold Spring Harbor Laboratory publications, New York, 1988, 511-52.

Furthermore, the C2orf18 polypeptide or functional fragments thereof used for the screening of agents or compounds that bind thereto may be bound to a carrier. Example of carriers that may be used for binding the polypeptides include insoluble polysaccharides, such as agarose, cellulose and dextran; and synthetic resins, such as polyacrylamide, polystyrene and silicon; preferably commercially available beads and plates (e.g., multi-well plates, biosensor chip, etc.) prepared from the above materials may be used. When using beads, they may be filled into a column. Alternatively, the use of magnetic beads is also known in the art, and enables to readily isolate the polypeptides and the test agents or compounds bound on the beads via magnetism.

The binding of a polypeptide to a carrier may be conducted according to routine methods, such as chemical bonding and physical adsorption. Alternatively, a polypeptide may be bound to a carrier via antibodies specifically recognizing the protein. Moreover, binding of a polypeptide to a carrier can also be conducted by means of interacting molecules, such as the combination of avidin and biotin.

Screening methods using such carrier-bound C2orf18 polypeptide or functional fragments thereof include, for example, the steps of contacting a test agent or compound to the carrier-bound polypeptide, incubating the mixture, washing the carrier, and detecting and/or measuring the agent or compound bound to the carrier. The binding may be carried out in buffer, for example, but are not limited to, phosphate buffer and Tris buffer, as long as the buffer does not inhibit the binding.

An exemplary screening method using such carrier-bound C2orf18 polypeptide or fragments thereof includes affinity chromatography. For example, the C2orf 18 polypeptide may be immobilized on a carrier of an affinity column, and a solution containing at least one test agent or compound is applied to the column. After loading the test agent or compound, the column is washed, and then the test agent or compound bound to the polypeptide is eluted with an appropriate buffer.

A biosensor using the surface plasmon resonance phenomenon may be used as a mean for detecting or quantifying the bound agent or compound in the present invention. When such a biosensor is used, the interaction between the C2orf18 polypeptide and a test agent or compound can be observed real-time as a surface plasmon resonance signal, using only a minute amount of the polypeptide and without labeling (for example, BIAcore, Pharmacia). Therefore, it is possible to evaluate the binding between the polypeptide and a test agent or compound using a biosensor such as BIAcore.

Methods of screening for molecules that bind to a specific protein among synthetic chemical compounds, or molecules in natural substance banks or a random phage peptide display library by exposing the specific protein immobilized on a carrier to the molecules, and methods of high-throughput screening based on combinatorial chemistry techniques (Wrighton et al., Science 1996, 273:458-64; Verdine, Nature 1996, 384:11-3) to isolate not only proteins but chemical compounds are also well-known to those skilled in the art. These methods can also be used for screening agents or compounds (including agonist and antagonist) that bind to the C2orf18 protein or fragments thereof.

When the test agent or compound is a protein, for example, West-Western blotting analysis (Skolnik et al., Cell 1991, 65:83-90) can be used for the present method. Specifically, a protein binding to the C2orf 18 polypeptide can be obtained by preparing first a cDNA library derived from cells, tissues, organs, or cultured cells (e.g., pancreatic cancer cells) expected to express at least one protein binding to the C2orf18 polypeptide using a phage vector (e.g., ZAP), expressing the proteins encoded by the vectors of the cDNA library on LB-agarose, fixing the expressed proteins on a filter, reacting the purified and labeled C2orf18 polypeptide with the above filter, and then detecting the plaques expressing proteins to which the C2orf18 polypeptide has bound according to the label of the C2orf 18 polypeptide.

Labeling substances such as radioisotope (e.g., ³H, ¹⁴C, ³²P, ³³P, ³⁵S, ¹²⁵I, ¹³¹I), enzymes (e.g., alkaline phosphatase, horseradish peroxidase, beta-galactosidase, alpha-glucosidase), fluorescent substances (e.g., fluorescein isothiosyanete (FITC), rhodamine) and biotin/avidin, may be used for the labeling of the C2orf18 polypeptide in the present method. When the protein is labeled with radioisotope, the detection or measurement can be carried out by liquid scintillation. Alternatively, when the protein is labeled with an enzyme, it can be detected or measured by adding a substrate of the enzyme to detect the enzymatic change of the substrate, such as generation of color, with absorptiometer. Further, in case where a fluorescent substance is used as the label, the bound protein may be detected or measured using fluorophotometer.

Moreover, the C2orf18 polypeptide bound to the protein can be detected or measured by utilizing an antibody that specifically binds to the C2orf 18 polypeptide, or a peptide or polypeptide (for example, GST) that is fused to the C2orf18 polypeptide. In case of using an antibody in the present screening, the antibody is preferably labeled with one of the labeling substances mentioned above, and detected or measured based on the labeling substance. Alternatively, the antibody against the C2orf18 polypeptide may be used as a primary antibody to be detected with a secondary antibody that is labeled with a labeling substance. Furthermore, the antibody bound to the C2orf 18 polypeptide in the present screening may be detected or measured using protein G or protein A column.

Alternatively, in another embodiment of the screening method of the present invention, two-hybrid system utilizing cells may be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton et al., Cell 1992, 68:597-612” and “Fields et al., Trends Genet. 1994, 10:286-92”). In two-hybrid system, the C2orf18 polypeptide or a fragment thereof is fused to the SRF-binding region or GAL4-binding region and expressed in yeast cells. A cDNA library is prepared from cells expected to express at least one protein binding to the C2orf18 polypeptide, such that the library, when expressed, is fused to the VP16 or GAL4 transcriptional activation region. The cDNA library is then introduced into the above yeast cells and the cDNA derived from the library is isolated from the positive clones detected (when a protein binding to the C2orf18 polypeptide is expressed in the yeast cells, the binding of the two activates a reporter gene, making positive clones detectable). A protein encoded by the cDNA can be prepared by introducing the cDNA isolated above to E. coli and expressing the protein.

As a reporter gene, for example, Ade2 gene, lacZ gene, CAT gene, luciferase gene and such can be used in addition to the HIS3 gene.

The agent or compound identified by this screening is a candidate for agonists or antagonists of the C2orf18 polypeptide. The term “agonist” refers to molecules that activate the function of the polypeptide by binding thereto. On the other hand, the term “antagonist” refers to molecules that inhibit the function of the polypeptide by binding thereto. Moreover, an agent or compound isolated by this screening as an antagonist is a candidate that inhibits the in vivo interaction of the C2orf 18 polypeptide with molecules (including nucleic acids (RNAs and DNAs) and proteins).

II-2. Identifying Agents or Compounds by Detecting Biological Activity of the C2orf18 Polypeptide

According to the present invention, the expression of the C2orf18 gene was shown to by crucial for the growth and/or survival of cells over-expressing C2orf18, such as cancer cells, specifically pancreatic cancer cells, more specifically PDAC cells. Therefore, agents or compounds that suppress or inhibit the expression or biological function of the translational product of the C2orf18 gene is considered to serve as a candidate agent or compound for inhibiting the cell growth or a candidate agent or compound for treating or preventing C2orf18 associating disease. Thus, the present invention also provides a method of screening for a candidate agent or compound for inhibiting the cell growth or a candidate agent or compound for treating or preventing C2orf18 associating disease, using the C2orf18 polypeptide or fragments thereof including the steps as follows:

a) contacting a test agent or compound with the C2orf 18 polypeptide or a functional fragment thereof; and
b) detecting the biological activity of the polypeptide or fragment of step (a).

According to the present invention, the therapeutic effect of the test agent or compound on inhibiting the cell growth or a candidate agent or compound for treating or preventing C2orf18 associating disease may be evaluated. Therefore, the present invention also provides a method of screening for a candidate agent or compound for inhibiting the cell growth or a candidate agent or compound for treating or preventing C2orf18 associating disease, using the C2orf18 polypeptide or fragments thereof including the steps as follows:

a) contacting a test agent or compound with the C2orf 18 polypeptide or a functional fragment thereof; and
b) detecting the biological activity of the polypeptide or fragment of step (a). and
c) correlating the biological activity of b) with the therapeutic effect of the test agent or compound.

In the present invention, the therapeutic effect may be correlated with the biological activity C2orf18 polypeptide or a functional fragment thereof. For example, when the test agent or compound suppresses or inhibits the biological activity C2orf18 polypeptide or a functional fragment thereof as compared to a level detected in the absence of the test agent or compound, the test agent or compound may identified or selected as the candidate agent or compound having the therapeutic effect. Alternatively, when the test agent or compound does not suppress or inhibit the biological activity C2orf18 polypeptide or a functional fragment thereof as compared to a level detected in the absence of the test agent or compound, the test agent or compound may identified as the agent or compound having no significant therapeutic effect.

Any polypeptide can be used for the screening so long as it has one biological activity of the C2orf18 polypeptide that can be used as an index in the present screening method. Since the C2orf18 polypeptide has the activity of promoting cell proliferation of cancer cells, biological activities of the C2orf18 polypeptide that can be used as an index for the screening include such cell-proliferating activity of the human C2orf18 polypeptide. For example, a human C2orf18 polypeptide can be used and polypeptides functionally equivalent thereto including functional fragments thereof can also be used. Such polypeptides may be expressed endogenously or exogenously by suitable cells.

When the biological activity to be detected in the present method is cell proliferation activity or anti-apotosis activity, it can be detected, for example, by preparing cells which express the C2orf18 polypeptide or a functional fragment thereof, culturing the cells in the presence of a test agent or compound, and determining the speed of cell proliferation, measuring the cell cycle and such, as well as by detecting wound-healing activity, conducting Matrigel invasion assay and measuring the colony forming activity.

According to an aspect of the present invention, the screening further includes, after the above step (b), the step of:

c) selecting the test agent or compound that suppresses the biological activity of the polypeptide as compared to the biological activity detected in the absence of the test agent or compound.

Furthermore, the candidate agent or compound can be confirmed the specificity for C2orf18 by comparing to the effect for non-C2orf18 expressing cell. If the candidate agent or compound can effect in the cell expressing C2orf18 and not effect in the cell no-expressing C2orf 18, the candidate agent or compound has specificity for C2orf 18. The agent or compound isolated by this screening is a candidate agent or compound for an antagonist of the C2orf 18 polypeptide, and thus, is a candidate agent or compound that inhibits the in vivo interaction of the polypeptide with molecules (including nucleic acids (RNAs and DNAs) and proteins).

In addition to the cell proliferation activity or the anti-aptosis activity, the C2orf18 protein has a binding activity to the ANT2 protein. Thus, the present invention also provides a method of screening for an agent or compound that inhibits the binding between C2orf 18 and ANT2. An agent or compound that inhibits the binding between C2orf18 and ANT2 is expected to suppress the proliferation of cancer cells, and thus is useful for treating or preventing cancer. Therefore, the present invention also provides a method for screening a candidate agent or compound that suppresses the proliferation of cancer cells, and a method for screening a candidate agent or compound for treating or preventing cancer.

More specifically, the method includes the steps of:

(a) contacting a C2orf18 protein with a ANT2 protein in the presence of an test agent or compound;

(b) detecting the level of binding between the C2orf18 and ANT2 proteins;

(c) comparing the binding level of the C2orf18 and ANT2 proteins with that detected in the absence of the test agent or compound; and

(d) selecting the test agent or compound that reduces the binding level of C2orf 18 and ANT2 proteins as an agent or compound that inhibits the binding between the C2orf18 and ANT2 proteins, i.e., a candidate agent or compound that may be used to suppress the proliferation of cancer cells and for treating or preventing cancer.

According to the present invention, the therapeutic effect of the test agent or compound on inhibiting the cell growth or a candidate agent or compound for treating or preventing C2orf18 associating disease may be evaluated. Therefore, the present invention also provides a method for screening a candidate agent or compound that suppresses the proliferation of cancer cells, and a method for screening a candidate agent or compound for treating or preventing cancer.

More specifically, the method includes the steps of:

(a) contacting a C2orf18 protein with a ANT2 protein in the presence of an test agent or compound;

(b) detecting the level of binding between the C2orf18 and ANT2 proteins;

(c) comparing the binding level of the C2orf18 and ANT2 proteins with that detected in the absence of the test agent or compound; and

d) correlating the binding level of c) with the therapeutic effect of the test agent or compound.

In the present invention, the therapeutic effect may be correlated with the binding level of the C2orf18 and ANT2 proteins. For example, when the test agent or compound reduces the binding level of C2orf18 and ANT2 proteins as compared to a level detected in the absence of the test agent or compound, the test agent or compound may identified or selected as the candidate agent or compound having the therapeutic effect. Alternatively, when the test agent or compound does not reduce the binding level of C2orf18 and ANT2 proteins as compared to a level detected in the absence of the test agent or compound, the test agent or compound may identified as the agent or compound having no significant therapeutic effect.

In the context of the present invention, “inhibition of binding” between two proteins refers to at least reducing the binding between the proteins. Thus, in some cases, the percentage of binding pairs in a sample will be decreased as compared to that in an appropriate (e.g., not treated with the test agent) control sample. The amount of proteins bound may be, e.g., less than 90%, 80%, 70%, 60%, 50%, 40%, 25%, 10%, 5%, 1% or less (e.g., 0%), of that in a control sample.

Herein, the C2orf 18 protein and ANT2 protein may include functional equivalents of these proteins as described above. The C2orf18 or ANT2 protein or functional equivalents thereof used in the screening can be prepared as a recombinant protein or a natural protein, by methods well known to those skilled in the art. The proteins may be obtained adopting any known genetic engineering methods for producing polypeptides (e.g., Morrison J., J Bacteriology 1977, 132: 349-51; Clark-Curtiss & Curtiss, Methods in Enzymology (eds. Wu et al.) 1983, 101: 347-62). For example, a recombinant protein can be prepared by inserting a DNA, which encodes the protein (for example, the DNA having the nucleotide sequence of SEQ ID NO: 11 (for C2orf18) or 25 (for ANT2)), into an appropriate expression vector, introducing the vector into an appropriate host cell, incubating the host cell in appropriate medium, obtaining the extract of the host cell, and purifying the protein by subjecting the extract to chromatography, for example, ion exchange chromatography, reverse phase chromatography, gel filtration, or affinity chromatography utilizing a column to which antibodies against the protein is fixed, or by combining more than one of aforementioned columns.

Also, when the protein useful in the context of the present invention is expressed within host cells (for example, animal cells and E. coli) as a fusion protein with glutathione-5-transferase protein or as a recombinant protein supplemented with multiple histidines, the expressed recombinant protein can be purified using a glutathione column or nickel column.

After purifying the fusion protein, it is also possible to exclude regions other than the objective protein by cutting with thrombin or factor-Xa as required.

A natural protein can be isolated by methods known to a person skilled in the art, for example, by contacting the affinity column, in which antibodies binding to the C2orf18 or ANT2 protein described above are bound, with the extract of tissues or cells expressing the protein. The antibodies can be polyclonal antibodies, monoclonal antibodies, or any modified antibodies so long as it binds to the C2orf18 or ANT2 protein.

The C2orf18 or ANT2 protein or functional equivalents thereof may also be produced in vitro adopting an in vitro translation system.

Further, partial peptides of the C2orf18 and ANT2 proteins may also be used for the invention so long as they retain their binding activity to each other. Such partial peptides can be produced by genetic engineering, by known methods of peptide synthesis, or by digesting the natural C2orf18 or ANT2 protein with an appropriate peptidase. For peptide synthesis, for example, solid phase synthesis or liquid phase synthesis may be used. Conventional peptide synthesis methods that can be adopted for the synthesis include:

1) Peptide Synthesis, Interscience, New York, 1966;

2) The Proteins, Vol. 2, Academic Press, New York, 1976;

3) Peptide Synthesis (in Japanese), Maruzen Co., 1975;

4) Basics and Experiment of Peptide Synthesis (in Japanese), Maruzen Co., 1985;

5) Development of Pharmaceuticals (second volume) (in Japanese), Vol. 14 (peptide synthesis), Hirokawa, 1991;

6) WO99/67288; and

7) Barany G. & Merrifield R. B., Peptides Vol. 2, “Solid Phase Peptide Synthesis”, Academic Press, New York, 1980, 100-118.

The polypeptides or fragments thereof may be further linked to other substances, so long as the polypeptides and fragments retain their original ability to bind to each other. Usable substances include: peptides, lipids, sugar and sugar chains, acetyl groups, natural and synthetic polymers, etc. These kinds of modifications may be performed to confer additional functions or to stabilize the polypeptide and fragments.

The C2orf18 and ANT2 polypeptides or functional equivalent thereof to be contacted in the presence of a test agent or compound can be, for example, purified polypeptides, soluble proteins, or fusion proteins fused with other polypeptides.

The screening methods of the present invention provide efficient and rapid identification of test agents or compounds that have a high probability of interfering with the association of C2orf18 with its binding partner ANT2. Generally, any method that determines the ability of a test agent or compound to interfere with such association is suitable for use with the present invention. For example, competitive and non-competitive inhibition assays in an ELISA format may be utilized. Control experiments should be performed to determine maximal binding capacity of system (e.g., contacting C2orf 18 with ANT2, and determining the amount of AN2 bound to C2orf18).

As a method for identifying agents or compounds that inhibit the binding between proteins, many methods well known by one skilled in the art can be used. Such identification can be carried out as an in vitro assay system, for example, in a cellular system. More specifically, first, either C2orf18 or its partner ANT2 is bound to a support, and the other protein is contacted together with a test agent or compound thereto. Next, the mixture is incubated, washed and the other protein bound to the support is detected and/or measured.

Example of supports that may be used for binding the proteins include insoluble polysaccharides, such as agarose, cellulose and dextran; and synthetic resins, such as polyacrylamide, polystyrene and silicon; preferably commercially available beads and plates (e.g., multi-well plates, biosensor chip, etc.) prepared from the above materials may be used. When using beads, they may be filled into a column. Alternatively, the use of magnetic beads is also known in the art, and enables to readily isolate proteins bound on the beads via magnetism.

The binding of a protein to a support may be conducted according to routine methods, such as chemical bonding and physical adsorption. Alternatively, a protein may be bound to a support via antibodies specifically recognizing the protein. Moreover, binding of a protein to a support can also be conducted by means of interacting molecules, such as the combination of avidin and biotin.

The binding between proteins is carried out in buffer, for example, but are not limited to, phosphate buffer and Tris buffer, as long as the buffer does not inhibit the binding between the proteins.

In the present invention, a biosensor using the surface plasmon resonance phenomenon may be used as a means for detecting or quantifying the bound protein. When such a biosensor is used, the interaction between the proteins can be observed real-time as a surface plasmon resonance signal, using only a minute amount of polypeptide and without labeling (for example, BIAcore, Pharmacia). Therefore, it is possible to evaluate the binding between the C2orf18 and ANT2 using a biosensor such as BIAcore.

Alternatively, either C2orf 18 or ANT2 may be labeled, and the label of the bound protein may be used to detect or measure the bound protein. Specifically, after pre-labeling one of the proteins, the labeled protein is contacted with the other protein in the presence of a test agent or compound, and then the bound proteins are detected or measured according to the label after washing.

Labeling substances such as radioisotope (e.g., ³H, ¹⁴C, ³²I, ³³P, ³⁵S, ¹²⁵I, ¹³¹I), enzymes (e.g., alkaline phosphatase, horseradish peroxidase, beta-galactosidase, beta-glucosidase), fluorescent substances (e.g., fluorescein isothiocyanate (FITC), fluorescein, Texas red, green fluorescent protein, and rhodamine), magnetic beads (e.g., DYNABEADS™), calorimetric labels (e.g., colloidal gold or colored glass or plastic (e.g., polystyrene, polypropylene, latex, etc.) beads), and biotin/avidin, may be used for the labeling of a protein in the present method. Patents teaching the use of such labels include U.S. Pat. Nos. 3,817,837; 3,850,752; 3,939,350; 3,996,345; 4,277,437; 4,275,149; and 4,366,241. However, the present invention is not restricted thereto and any label detectable by spectroscopic, photochemical, biochemical, immunochemical, electrical, optical or chemical means may be used.

When the protein is labeled with radioisotope, the detection or measurement can be carried out by liquid scintillation. Alternatively, proteins labeled with enzymes can be detected or measured by adding a substrate of the enzyme to detect the enzymatic change of the substrate, such as generation of color, with absorptiometer. Further, in case where a fluorescent substance is used as the label, the bound protein may be detected or measured using fluorophotometer.

Furthermore, the binding in the present screening method can be also detected or measured using an antibody against C2orf18 or ANT2. For example, after contacting C2orf 18 immobilized on a support with a test agent or compound and ANT2, the mixture is incubated and washed, and detection or measurement can be conducted using an antibody against ANT2. Alternatively, ANT2 may be immobilized on a support, and an antibody against C2orf18 may be used as the antibody.

When using an antibody in the present screening, the antibody is preferably labeled with one of the labeling substances mentioned above, and detected or measured based on the labeling substance. Alternatively, the antibody against the C2orf18 or ANT2 may be used as a primary antibody to be detected with a secondary antibody that is labeled with a labeling substance. Furthermore, the antibody bound to the protein in the screening of the present invention may be detected or measured using protein G or protein A column.

Alternatively, in another embodiment of the identification method of the present invention, a two-hybrid system utilizing cells may be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 1992, 68: 597-612”, “Fields and Sternglanz, Trends Genet. 1994, 10: 286-92”). In the two-hybrid system, for example, C2orf 18 is fused to the SRF-binding region or GAL4-binding region and expressed in yeast cells. ANT2 is fused to the VP16 or GAL4 transcriptional activation region and also expressed in the yeast cells in the existence of a test agent or compound. Alternatively, ANT2 may be fused to the SRF-binding region or GAL4-binding region, and C2orf18 to the VP16 or GAL4 transcriptional activation region. When the test agent or compound does not inhibit the binding between C2orf18 and ANT2, the binding of the two activates a reporter gene, making positive clones detectable. As a reporter gene, for example, Ade2 gene, lacZ gene, CAT gene, luciferase gene and such can be used besides HIS3 gene.

Herein, the binding level between C2orf18 and ANT2 can be also measured as any change occurring after the binding of C2orf18 and ANT2. Specifically, such screening can be performed by contacting a test agent or compound with a cell that expresses C2orf18 and ANT2, such as J82 or UMUC cells. For example, the suppression of cell proliferation may be detected to determine the influence of a test agent or compound on the binding of C2orf18 and ANT2.

1. Competitive Assay Format

Competitive assays may be used for screening test agents or compounds of the present invention. By way of example, a competitive ELISA format may include C2orf 18 (or ANT2) bound to a solid support. The bound C2orf 18 (or ANT2) would be incubated with ANT2 (or C2orf18) and a test agent or compound. After sufficient time to allow the test agent or compound and/or ANT2 (or C2orf18) to bind C2orf18 (or ANT2), the substrate would be washed to remove unbound material. The amount of ANT2 bound to C2orf 18 is then determined. This may be accomplished in any of a variety of ways known in the art, for example, by using ANT2 (or C2orf18) species tagged with a detectable label, or by contacting the washed substrate with a labeled antibody against ANT2 (or C2orf18). The amount of ANT2 (or C2orf18) bound to C2orf18 (or ANT2) will be inversely proportional to the ability of the test agent or compound to interfere with the association of C2orf18 to ANT2. The standard methods including antibodies and labels are described in Harlow & Lane, Antibodies, A Laboratory Manual (1988).

In a variation, C2orf 18 (or ANT2) is labeled with an affinity tag. Labeled C2orf 18 (or ANT2) is then incubated with a test agent or compound and ANT2 (or C2orf18), then immunoprecipitated. The immunoprecipitate is then subjected to Western blotting using an antibody against ANT2 (or C2orf18). As with the previous competitive assay format, the amount of ANT2 (or C2orf18) found associated with C2orf18 (or ANT2) is inversely proportional to the ability of the test agent or compound to interfere with the association of C2orf18 and ANT2.

2. Non-Competitive Assay Format

Non-competitive binding assays may also find utility as an initial screen for testing agent or compound libraries constructed in a format that is not readily amenable to screening using competitive assays, such as those described herein. An example of such a library is a phage display library (see, e.g., Barrett et al., Anal Biochem 1992, 204: 357-64).

Phage libraries find utility in being able to produce quickly working quantities of large numbers of different recombinant peptides. Phage libraries do not lend themselves to competitive assays of the invention, but can be efficiently screened in a non-competitive format to determine which recombinant peptide as a test agent or compound binds C2orf18 or ANT2. Test agents or compounds identified in a non-competitive format can then be produced by any methods well-known in the art and further screened using a competitive assay format. Production and screening of phage and cell display libraries are well-known in the art and discussed in, for example, Ladner et al., WO 88/06630; Fuchs et al., Biotechnology 1991, 9: 1369-72; Goward et al., TIBS 1993, 18: 136-40; Charbit et al., EMBO J. 1986, 5: 3029-37; Cull et al., PNAS USA 1992, 89: 1865-9; Cwirla et al., PNAS USA 1990, 87: 6378-82.

An exemplary non-competitive assay would follow an analogous procedure to the one described for the competitive assay, without the addition of one of the components (C2orf18 or ANT2). However, as non-competitive formats determine test agents or compounds binding to C2orf18 or ANT2, the ability of a test agent or compound to bind both C2orf18 and ANT2 needs to be determined for each candidate. Thus, by way of example, binding of the test agent or compound to immobilized C2orf18 may be determined by washing away unbound the test agent or compound; eluting bound the test agent or compound from the support, followed by analysis of the eluate; e.g., by mass spectroscopy, protein determination (Bradford or Lowry assay, or Abs. at 280 nm determination.). Alternatively, the elution step may be eliminated and binding of the test agent or compound may be determined by monitoring changes in the spectroscopic properties of the organic layer at the support surface. Methods for monitoring spectroscopic properties of surfaces include, but are not limited to, absorbance, reflectance, transmittance, birefringence, refractive index, diffraction, surface plasmon resonance, ellipsometry, resonant mirror techniques, grating coupled waveguide techniques and multipolar resonance spectroscopy, all of which are known to those of skill in the art. A labeled test agent or compound may also be used in the assay to eliminate need for an elution step. In this instance, the amount of label associated with the support after washing away unbound material is directly proportional to test agent or compound binding.

A number of well-known robotic systems have been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett Packard, Palo Alto, Calif.), which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

According to an aspect of the present invention, the components necessary for the present screening methods may be provided as a kit for screening agents or compounds that inhibit the binding between C2orf 18 and ANT2, or agents or compounds that suppress proliferation of pancreatic cancer cells, or agents or compounds for treating or preventing pancreatic cancer. The kit may contain, for example, the C2orf18 polypeptide or a function equivalent thereof, and/or ANT2 polypeptide or a functional equivalent thereof. Further, the kit may include control reagents (positive and/or negative), detectable labels, reaction buffers, cell culture medium, containers required for the screening, instructions (e.g., written, tape, VCR, CD-ROM, etc.) for carrying out the method, and so on. The components and reagents may be packaged in separate containers.

III. Nucleotide Based Screening Methods

III-1. Screening Method Using C2orf 18 Gene

As discussed in detail above, by controlling the expression level of the C2orf18 gene, one can control the cell growth or the onset and progression of C2orf18 associating disease. Thus, candidate agents or compounds that may be used in the inhibition of cell growth or the treatment or prevention of C2orf18 associating disease can be identified through screenings that use the expression levels of C2orf18 gene as indices. In the context of the present invention, such screening may include, for example, the following steps:

a) contacting a test agent or compound with a cell expressing the C2orf18 gene;
b) detecting the expression level of the C2orf18 gene; and
c) selecting the test agent or compound that reduces the expression level of the C2orf18 gene as compared to a level detected in the absence of the test agent or compound.

According to the present invention, the therapeutic effect of the test agent or compound on inhibiting the cell growth or a candidate agent or compound for treating or preventing C2orf18 associating disease may be evaluated. Therefore, the present invention also provides a method for screening a candidate agent or compound that suppresses the proliferation of cancer cells, and a method for screening a candidate agent or compound for treating or preventing C2orf18 associating disease. In the context of the present invention, such screening may include, for example, the following steps:

a) contacting a test agent or compound with a cell expressing the C2orf18 gene;
b) detecting the expression level of the C2orf18 gene; and
c) correlating the expression level of b) with the therapeutic effect of the test agent or compound.

In the present invention, the therapeutic effect may be correlated with the expression level of the C2orf 18 gene. For example, when the test agent or compound reduces the expression level of the C2orf18 gene as compared to a level detected in the absence of the test agent or compound, the test agent or compound may identified or selected as the candidate agent or compound having the therapeutic effect. Alternatively, when the test agent or compound does not reduce the expression level of the C2orf18 gene as compared to a level detected in the absence of the test agent or compound, the test agent or compound may identified as the agent or compound having no significant therapeutic effect.

Morespecically, said expression level may be detected by methods select from the group consisting of:

(a) detecting the amount of the mRNA encoding the C2orf 18 polypeptide, or functional equivalent thereof;

(b) detecting the amount of the C2orf18 polypeptide, or functional equivalent thereof; and

(c) detecting the biological activity of the C2orf18 polypeptide, or functional equivalent thereof.

Preferably, a cell proliferative activity of the cell expressing C2orf18 polypeptide may be detected as said biological activity.

In the present invention, the cell is characterized by over-expression of C2orf18, such as cancer cell, e.g. pancreatic cancer cell, pancreatic ductal adenocarcinoma (PDAC) cell. The C2orf18 associating disease is characterized by over-expression of C2orf18, such as cancer, e.g. pancreatic cancer, specifically pancreatic ductal adenocarcinoma (PDAC). An agent or compound that inhibits the expression of the C2orf18 gene can be identified by contacting a cell expressing the C2orf18 gene with a test agent or compound and then determining the expression level of the C2orf18 gene. Naturally, the identification may also be performed using a population of cells that express the gene in place of a single cell. A decreased expression level detected in the presence of a test agent or compound as compared to the expression level in the absence of the test agent or compound indicates the test agent or compound as being an inhibitor of the C2orf18 gene, suggesting the possibility that the test agent or compound is useful for inhibiting cancer, thus a candidate agent or compound to be used for the treatment or prevention of cancer.

The expression level of a gene can be estimated by methods well known to one skilled in the art. The expression level of the C2orf18 gene can be, for example, determined the method described in ‘EXAMPLES’.

The cell or the cell population used for such identification may be any cell or any population of cells so long as it expresses the C2orf18 gene. For example, the cell or the cell population may be or contain an epithelial cell derived from a tissue. Alternatively, the cell or the cell population may be or contain an immortalized cell derived from a cancerous cell, including those derived from pancreatic cancer, e.g. PDAC. Cells expressing the C2orf18 gene include, for example, cell lines established from cancers (e.g. MIA-PaCA2). Furthermore, the cell or the cell population may be or contain a cell which has been transfected with C2orf 18 gene.

The present method permits the screening of various agents or compounds and is particularly suited for identifying functional nucleic acid molecules including antisense RNA, siRNA, and such.

III-2. Screening Method Using Transcriptional Regulatory Region of C2Orf18 Gene

According to another aspect, the present invention provides a method which includes the following steps of:

a) contacting a test agent or compound with a cell into which a vector, composed of the transcriptional regulatory region of the C2orf 18 gene and a reporter gene that is expressed under the control of the transcriptional regulatory region, has been introduced;

b) detecting the expression or activity of said reporter gene; and

c) selecting the test agent or compound that reduces the expression or activity of said reporter gene as compared to a level detected in the absence of the test agent or compound.

According to the present invention, the therapeutic effect of the test agent or compound on inhibiting the cell growth or a candidate agent or compound for treating or preventing C2orf18 associating disease may be evaluated. Therefore, the present invention also provides a method for screening a candidate agent or compound that suppresses the proliferation of cancer cells, and a method for screening a candidate agent or compound for treating or preventing C2orf18 associating disease.

In the context of the present invention, such screening may include, for example, the following steps:

According to another aspect, the present invention provides a method which includes the following steps of:

a) contacting a test agent or compound with a cell into which a vector, composed of the transcriptional regulatory region of the C2orf 18 gene and a reporter gene that is expressed under the control of the transcriptional regulatory region, has been introduced;

b) detecting the expression or activity of said reporter gene; and

c) correlating the expression level of b) with the therapeutic effect of the test agent or compound.

In the present invention, the therapeutic effect may be correlated with the expression or activity of said reporter gene. For example, when the test agent or compound reduces the expression or activity of said reporter gene as compared to a level detected in the absence of the test agent or compound, the test agent or compound may identified or selected as the candidate agent or compound having the therapeutic effect. Alternatively, when the test agent or compound does not reduce the expression or activity of said reporter gene as compared to a level detected in the absence of the test agent or compound, the test agent or compound may identified as the agent or compound having no significant therapeutic effect.

Suitable reporter genes and host cells are well known in the art. The reporter construct required for the screening can be prepared using the transcriptional regulatory region of the C2orf18 gene, which can be obtained as a nucleotide segment containing the transcriptional regulatory region from a genome library based on the nucleotide sequence information of the gene.

The transcriptional regulatory region may be, for example, the promoter sequence of the C2orf18 gene. The reporter construct required for the screening can be prepared by connecting reporter gene sequence to the transcriptional regulatory region of C2orf18 gene. The transcriptional regulatory region of C2orf 18 gene herein is the region from start codon to at least 500 bp upstream, preferably 1000 bp, more preferably 5000 or 10000 bp upstream. A nucleotide segment containing the transcriptional regulatory region can be isolated from a genome library or can be propagated by PCR. Methods for identifying a transcriptional regulatory region, and also assay protocol are well known (Molecular Cloning third edition chapter 17, 2001, Cold Springs Harbor Laboratory Press). The vector containing the said reporter construct is infected to host cells and the expression level or activity of the reporter gene is detected by method well known in the art (e.g., using luminometer, absorption spectrometer, flow cytometer and so on). “reduces the expression level or activity” as defined herein are preferably at least 10% reduction of the expression level or activity of the reporter gene in comparison with in absence of the compound, more preferably at least 25%, 50% or 75% reduction and most preferably at least 95% reduction.

When a cell(s) transfected with a reporter gene that is operably linked to the regulatory sequence (e.g. promoter sequence) of the C2orf18 gene is used, an test agent or compound can be identified as inhibiting or enhancing the expression of the C2orf18 gene through detecting the expression level of the reporter gene product. As a reporter gene, for example, Ade2 gene, lacZ gene, CAT gene, luciferase gene, HIS3 gene, and such well-known in the art can be used. Methods for detection of the expression of these genes are well known in the art.

III-3. Selecting Therapeutic Agents or Compounds that are Appropriate for a Particular Individual

Differences in the genetic makeup of individuals can result in differences in their relative abilities to metabolize various drugs. An agent or compound that is metabolized in a subject to act as an anti-tumor agent or compound can manifest itself by inducing a change in a gene expression pattern in the subject's cells from that characteristic of a cancerous state to a gene expression pattern characteristic of a non cancerous state. Accordingly, the C2orf18 gene differentially expressed between cancerous and non-cancerous cells disclosed herein allow for a putative therapeutic or prophylactic inhibitor of cancer to be tested in a test cell population from a selected subject in order to determine if the agent or compound is a suitable inhibitor of cancer in the subject.

To identify an inhibitor of cancer that is appropriate for a specific subject, a test cell population from the subject is exposed to a candidate therapeutic agent or compound, and the expression of C2orf18 gene is determined. In the context of the method of the present invention, test cell populations contain cancer cells expressing the C2orf18 gene. Preferably, the test cell is an epithelial cell.

Specifically, a test cell population may be incubated in the presence of a candidate therapeutic agent or compound and the expression level of the C2orf18 gene in the test cell population may be measured and compared to one or more reference profiles, e.g., a cancerous reference expression profile, a non-cancerous reference expression profile or a reference expression profile in the absence of the therapeutic agent or compound.

A decrease in the expression level of the C2orf18 gene in a test cell population contacted with a therapeutic agent or compound relative to a reference cell population containing cancer cells or a reference cell population in the absence of the agent or compound, indicates that the therapeutic agent or compound has therapeutic potential in the subject from which the test cell population is derived.

Compositions for Inhibiting Cell Growth or Treating or Preventing Cancers:

The agents identified by any of the screening methods of the present invention, double-stranded molecules against the C2orf18 gene, and antibodies against the C2orf 18 polypeptide inhibit or suppress the expression of the C2orf 18 gene, or the biological activity of the C2orf 18 polypeptide and thus inhibit cell proliferation. Thus, the present invention provides compositions for inhibiting the cell growth or composition for treating or preventing C2orf18 associating disease, which compositions include agents identified by any of the screening methods of the present invention, e.g. double-stranded molecules against the C2orf18 gene, or antibodies against the C2orf18 polypeptide, peptide mimetics, conpounds or combination thereof. In the present invention, the cell is characterized by over-expression of C2orf 18, such as cancer cell, e.g. pancreatic cancer cell, specifically pancreatic ductal adenocarcinoma (PDAC) cell. The C2orf18 associating disease is characterized by over-expression of C2orf18, such as cancer, e.g. pancreatic cancer, specifically pancreatic ductal adenocarcinoma (PDAC). The present compositions can be used for treating or preventing C2orf18 associating disease, such as a cancer, specifically pancreatic cancer, e.g. PDAC.

The compositions may be used as pharmaceuticals for humans and other mammals, such as mice, rats, guinea-pigs, rabbits, cats, dogs, sheep, pigs, cattle, monkeys, baboons, and chimpanzees.

In the context of the present invention, suitable pharmaceutical formulations for the active ingredients of the present invention detailed below (including screened agents, antisense nucleic acids, double-stranded molecules, antibodies, etc.) include those suitable for oral, rectal, nasal, topical (including buccal and sub-lingual), vaginal or parenteral (including intramuscular, subcutaneous and intravenous) administration, or for administration by inhalation or insufflation. Preferably, administration is intravenous. The formulations are optionally packaged in discrete dosage units.

Pharmaceutical formulations suitable for oral administration include capsules, microcapsules, cachets and tablets, each containing a predetermined amount of active ingredient. Suitable formulations also include powders, elixirs, granules, solutions, suspensions and emulsions. The active ingredient is optionally administered as a bolus electuary or paste. Alternatively, according to needs, the pharmaceutical composition may be administered non-orally, in the form of injections of sterile solutions or suspensions with water or any other pharmaceutically acceptable liquid. For example, the active ingredients of the present invention can be mixed with pharmaceutically acceptable carriers or media, specifically, sterilized water, physiological saline, plantoils, emulsifiers, suspending agents, surfactants, stabilizers, flavoring agents, excipients, vehicles, preservatives, binders, and such, in a unit dose form required for generally accepted drug implementation. The amount of active ingredient contained in such a preparation makes a suitable dosage within the indicated range acquirable.

Examples of additives that can be admixed into tablets and capsules include, but are not limited to, binders, such as gelatin, corn starch, tragacanth gum and arabic gum; excipients, such as crystalline cellulose; swelling agents, such as corn starch, gelatin and alginic acid; lubricants, such as magnesium stearate; sweeteners, such as sucrose, lactose or saccharin; and flavoring agents, such as peppermint, Gaultheria adenothrix oil and cherry. A tablet may be made by compression or molding, optionally with one or more formulational ingredients. Compressed tablets may be prepared by compressing in a suitable machine the active ingredients in a free-flowing form such as powder or granules, optionally mixed with a binder, lubricant, inert diluent, lubricating, surface active or dispersing agent. Molded tablets may be made via molding in a suitable machine a mixture of the powdered compound moistened with an inert liquid diluent. The tablets may be coated according to methods well known in the art. The tablets may optionally be formulated so as to provide slow or controlled release of the active ingredient in vivo. A package of tablets may contain one tablet to be taken on each of the month. Furthermore, when the unit-dosage form is a capsule, a liquid carrier, such as oil, can be further included in addition to the above ingredients.

Oral fluid preparations may be in the form of, for example, aqueous or oily suspensions, solutions, emulsions, syrups or elixirs, or may be presented as a dry product for reconstitution with water or other suitable vehicle prior to use. Such liquid preparations may contain conventional additives such as suspending agents, emulsifying agents, non-aqueous vehicles (which may include edible oils) or preservatives.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

Moreover, sterile composites for injection can be formulated following normal drug implementations using vehicles, such as distilled water, suitable for injection. Physiological saline, glucose, and other isotonic liquids, including adjuvants, such as Dsorbitol, D-mannose, D-mannitol, and sodium chloride, can be used as aqueous solutions for injection. These can be used in conjunction with suitable solubilizers, such as alcohol, for example, ethanol; polyalcohols, such as propylene glycol and polyethylene glycol; and non-ionic surfactants, such as Polysorbate 80 (TM) and HCO-50.

Sesame oil or soy-bean oil can be used as an oleaginous liquid, which may be used in conjunction with benzyl benzoate or benzyl alcohol as a solubilizer, and may be formulated with a buffer, such as phosphate buffer and sodium acetate buffer; a painkiller, such as procaine hydrochloride; a stabilizer, such as benzyl alcohol and phenol; and/or an anti-oxidant. A prepared injection may be filled into a suitable ampoule.

Formulations for rectal administration include suppositories with standard carriers such as cocoa butter or polyethylene glycol. Formulations for topical administration in the mouth, for example, buccally or sublingually, include lozenges, which contain the active ingredient in a flavored base such as sucrose and acacia or tragacanth, and pastilles including the active ingredient in a base such as gelatin, glycerin, sucrose or acacia. For intra-nasal administration of an active ingredient, a liquid spray or dispersible powder or in the form of drops may be used. Drops may be formulated with an aqueous or non-aqueous base also including one or more dispersing agents, solubilizing agents or suspending agents.

For administration by inhalation the compositions are conveniently delivered from an insufflator, nebulizer, pressurized packs or other convenient means of delivering an aerosol spray. Pressurized packs may include a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the compositions may take the form of a dry powder composition, for example, a powder mix of an active ingredient and a suitable powder base such as lactose or starch. The powder composition may be presented in unit dosage form in, for example, capsules, cartridges, gelatin or blister packs from which the powder may be administered with the aid of an inhalator or insufflators.

Other formulations include implantable devices and adhesive patches; which release a therapeutic agent.

When desired, the above-described formulations, adapted to give sustained release of the active ingredient, may be employed. The pharmaceutical compositions may also contain other active ingredients such as antimicrobial agents, immunosuppressants or preservatives.

It should be understood that in addition to the ingredients particularly mentioned above, the formulations of this invention may include other agents conventional in the art having regard to the type of formulation in question, for example, those suitable for oral administration may include flavoring agents.

Preferred unit dosage formulations are those containing an effective dose, as recited below under the item of ‘Method Of Treating A C2orf18-Associating Disease’, of each of the active ingredients of the present invention or an appropriate fraction thereof.

I. Compositions Including a Double-Stranded Molecule

The present invention provides compositions for preventing cell growth and/or treating or preventing a C2orf18 associating disease including any of the double-stranded molecules described above or selected by the above-described screening methods of the present invention. A double-stranded molecule of the present invention can be adapted for use to inhibit cell growth and prevent or treat C2orf18 associating disease.

In one embodiment, a composition composed of one or more double-stranded molecules of the present invention can be encapsulated in a delivery vehicle, e.g. liposomes, for administration to a subject, carriers and diluents and their salts, and/or can be present in pharmaceutically acceptable formulations. Methods for the delivery of nucleic acid molecules are described in Akhtar S & Juliano R L. Trends Cell Biol. 1992 May; 2(5):139-44.; Delivery Strategies for Antisense Oligonucleotide Therapeutics, ed. Akhtar, 1995; Maurer N, et al., Mol Membr Biol. 1999 January-March; 16(1):129-40.; Hofland & Huang. Handb Exp Pharmacol. 1999 137:165-192. It further describes the general methods for delivery of nucleic acid molecules (U.S. Pat. No. 6,395,713 and WO 199402595). These protocols can be utilized for the delivery of virtually any double-stranded molecule. Double-stranded molecules can be administered to cells by a variety of methods known to those of skill in the art, including but not restricted to, encapsulation in liposomes, by iontophoresis, or by incorporation into other vehicles, such as biodegradable polymers, hydrogels, cyclodextrins (see for example Gonzalez H, et al., Bioconjug Chem. 1999 November-December; 10(6):1068-74.; WO 03/47518 and WO 03/46185), poly (lactic-co-glycolic) acid (PLGA) and PLCA microspheres (see for example U.S. Pat. No. 6,447,796 and US 2002130430), biodegradable nanocapsules, and bioadhesive microspheres, or by proteinaceous vectors (WO 200053722). In another embodiment, the double-stranded molecules of the present invention can also be formulated or complexed with polyethyleneimine and derivatives thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives. In one embodiment, the double-stranded molecules of the present invention are formulated as described in US 20030077829 (i.e. lipid-based formulations), incorporated by reference herein in its entirety.

The double-stranded molecules of the present invention can also be administered to a subject in combination with other therapeutic compounds to increase the overall therapeutic effect. The use of multiple compounds to treat an indication can increase the beneficial effects while reducing the presence of side effects.

In another embodiment, the present invention also provides the use of the double-stranded molecules of the present invention in manufacturing a pharmaceutical composition for use in treating or preventing a cancer expressing the C2orf18 gene. For example, the present invention relates to a use of double-stranded molecules inhibiting the expression of the C2orf18 gene in a cell, which molecule includes a sense strand and an antisense strand complementary thereto, hybridized to each other to form the double-stranded molecule and targets to a sequence of SEQ ID NOs: 7 or 8, for manufacturing a pharmaceutical composition for use in treating a cancer expressing the C2orf 18 gene.

In another embodiment, the present invention also provides the double-stranded nucleic acid molecules of the present invention for use in treating or preventing a cancer expressing the C2orf18 gene. Alternatively, the present invention further provides a method or process for manufacturing a pharmaceutical composition for treating a cancer expressing the C2orf18 gene, wherein the method or process includes the step of formulating a pharmaceutically or physiologically acceptable carrier with a double-stranded molecule inhibiting the expression of the C2orf18 gene in a cell, which molecule includes a sense strand and an antisense strand complementary thereto, hybridized to each other to form the double-stranded nucleic acid molecule and targets to a sequence of SEQ ID NOs: 7 or 8 as active ingredients.

In another embodiment, the present invention also provides a method or process for manufacturing a pharmaceutical composition for treating a cancer expressing the C2orf18 gene, wherein the method or process includes the step of admixing an active ingredient with a pharmaceutically or physiologically acceptable carrier, wherein the active ingredient is a double-stranded nucleic acid molecule inhibiting the expression of a C2orf18 gene in a cell, which molecule includes a sense strand and an antisense strand complementary thereto, hybridized to each other to form the double-stranded nucleic acid molecule and targets to a sequence of SEQ ID NOs: 7 or 8.

II. Compositions Including Antisense Nucleic Acids

Antisense nucleic acids corresponding to the nucleotide sequence of the C2orf18 gene can be used to reduce the expression level of the gene, which is up-regulated in cancer cells, are useful for the inhibition of cell growth and the treatment of cancer, and thus are also encompassed by the present invention. An antisense nucleic acid acts by binding to the nucleotide sequence of the C2orf18 gene, or mRNAs corresponding thereto, thereby inhibiting the transcription or translation of the gene, promoting the degradation of the mRNAs, and/or inhibiting the expression of the protein encoded by the gene. Thus, as a result, an antisense nucleic acid inhibits the C2orf18 protein to function in the cancerous cell. Herein, the phrase “antisense nucleic acids” refers to nucleotides that specifically hybridize to a target sequence and includes not only nucleotides that are entirely complementary to the target sequence but also that includes mismatches of one or more nucleotides. For example, the antisense nucleic acids of the present invention include polynucleotides that have a homology of at least 70% or higher, preferably of at least 80% or higher, more preferably of at least 90% or higher, even more preferably of at least 95% or higher over a span of at least 15 continuous nucleotides of the C2orf18 gene or the complementary sequence thereof. Algorithms known in the art can be used to determine such homology.

Antisense nucleic acids of the present invention act on cells producing proteins encoded by the C2orf 18 gene by binding to the DNA or mRNA of the gene, inhibiting their transcription or translation, promoting the degradation of the mRNA, and inhibiting the expression of the protein, finally inhibiting the protein to function.

Antisense nucleic acids of the present invention can be made into an external preparation, such as a liniment or a poultice, by admixing it with a suitable base material which is inactive against the nucleic acids.

Also, as needed, the antisense nucleic acids of the present invention can be formulated into tablets, powders, granules, capsules, liposome capsules, injections, solutions, nose-drops and freeze-drying agents by adding excipients, isotonic agents, solubilizers, stabilizers, preservatives, pain-killers, and such. An antisense-mounting medium can also be used to increase durability and membrane-permeability. Examples include, but are not limited to, liposomes, poly-L-lysine, lipids, cholesterol, lipofectin, or derivatives of these. These can be prepared by following known methods.

The antisense nucleic acids of the present invention inhibit the expression of the C2orf18 protein and are useful for suppressing the biological activity of the protein. In addition, expression-inhibitors, including antisense nucleic acids of the present invention, are useful in that they can inhibit the biological activity of the C2orf 18 protein.

The antisense nucleic acids of present invention include modified oligonucleotides. For example, thioated oligonucleotides may be used to confer nuclease resistance to an oligonucleotide.

In another embodiment, the present invention also provides the use of the antisense nucleic acids of the present invention in manufacturing a pharmaceutical composition for use in treating a cancer expressing the C2orf18 gene.

In another embodiment, the present invention also provides the antisense nucleic acids of the present invention for use in treating or preventing a cancer expressing the C2orf 18 gene.

Alternatively, the present invention further provides a method or process for manufacturing a pharmaceutical composition for treating a cancer expressing the C2orf18 gene, wherein the method or process includes the step of formulating a pharmaceutically or physiologically acceptable carrier with the antisense nucleic acids of the present invention as active ingredients.

In another embodiment, the present invention also provides a method or process for manufacturing a pharmaceutical composition for treating a cancer expressing the C2orf18 gene, wherein the method or process includes step for admixing an active ingredient with a pharmaceutically or physiologically acceptable carrier, wherein the active ingredient is the antisense nucleic acids of the present invention.

III. Compositions Including Antibodies

The function of a gene product of the C2orf 18 gene which is over-expressed in cancer can be inhibited by administering a compound that binds to or otherwise inhibits the function of the gene products. An antibody against the C2orf18 polypeptide can be mentioned as such a compound and can be used as the active ingredient of cell growth inhibitor or a pharmaceutical composition for treating or preventing C2orf18 associating disease.

The present invention relates to the use of antibodies against a protein encoded by the C2orf18 gene, or fragments of the antibodies. As used herein, the term “antibody” refers to the above mentioned meaning.

An antibody may be modified by conjugation with a variety of molecules, such as polyethylene glycol (PEG). The present invention includes such modified antibodies. The modified antibody can be obtained by chemically modifying an antibody. Such modification methods are conventional in the field.

Alternatively, the antibody used for the present invention may be a chimeric antibody having a variable region derived from a non-human antibody against the C2orf 18 polypeptide and a constant region derived from a human antibody, or a humanized antibody, composed of a complementarity determining region (CDR) derived from a non-human antibody, a frame work region (FR) and a constant region derived from a human antibody. Such antibodies can be prepared by using known technologies. Humanization can be performed by substituting rodent CDRs or CDR sequences for the corresponding sequences of a human antibody (see e.g., Verhoeyen et al., Science 1988, 239:1534-6). Accordingly, such humanized antibodies are chimeric antibodies, wherein substantially less than an intact human variable domain has been substituted by the corresponding sequence from a non-human species.

Complete human antibodies including human variable regions in addition to human framework and constant regions can also be used. Such antibodies can be produced using various techniques known in the art. For example, in vitro methods involve use of recombinant libraries of human antibody fragments displayed on bacteriophage (e.g., Hoogenboom et al., J Mol Biol 1992, 227:381-8). Similarly, human antibodies can be made by introducing human immunoglobulin loci into transgenic animals, e.g., mice in which the endogenous immunoglobulin genes have been partially or completely inactivated. This approach is described, e.g., in U.S. Pat. Nos. 6,150,584; 5,545,807; 5,545,806; 5,569,825; 5,625,126; 5,633,425; and 5,661,016.

When the obtained antibody is to be administered to the human body (antibody treatment), a human antibody or a humanized antibody is preferable for reducing immunogenicity.

Antibodies obtained as above may be purified to homogeneity. For example, the separation and purification of the antibody can be performed according to separation and purification methods used for general proteins. For example, the antibody may be separated and isolated by the appropriately selected and combined use of column chromatographies, such as affinity chromatography, filter, ultrafiltration, salting-out, dialysis, SDS polyacrylamide gel electrophoresis, isoelectric focusing, and others (Antibodies: A Laboratory Manual. Ed Harlow and David Lane, Cold Spring Harbor Laboratory (1988)), but are not limited thereto. A protein A column and protein G column can be used as the affinity column. Exemplary protein A columns to be used include, for example, Hyper D, POROS, and Sepharose F.F. (Pharmacia).

Exemplary chromatography, with the exception of affinity includes, for example, ion-exchange chromatography, hydrophobic chromatography, gel filtration, reverse-phase chromatography, adsorption chromatography, and the like (Strategies for Protein Purification and Characterization: A Laboratory Course Manual. Ed Daniel R. Marshak et al., Cold Spring Harbor Laboratory Press (1996)). The chromatographic procedures can be carried out by liquid-phase chromatography, such as HPLC and FPLC.

In another embodiment, the present invention also provides the use of the antibody of the present invention in manufacturing a pharmaceutical composition for use in treating a cancer expressing the C2orf18 gene.

In another embodiment, the present invention also provides the antibody of the present invention for use in treating or preventing a cancer expressing the C2orf18 gene. Alternatively, the present invention further provides a method or process for manufacturing a pharmaceutical composition for treating a cancer expressing the C2orf18 gene, wherein the method or process includes step for formulating a pharmaceutically or physiologically acceptable carrier with the antibody of the present invention as active ingredients.

In another embodiment, the present invention also provides a method or process for manufacturing a pharmaceutical composition for treating a cancer expressing the C2orf18 gene, wherein the method or process includes step for admixing an active ingredient with a pharmaceutically or physiologically acceptable carrier, wherein the active ingredient is the antibody of the present invention.

Methods of Inhibiting Cell Growth:

Knockdown of endogenous C2orf18 by siRNA in pancreas cancer cell lines resulted in drastic suppression of the C2orf18 over-expressing cell growth, suggesting its essential role in maintaining viability of these cells. Therefore, the present invention relates to inhibiting cell growth by inhibiting expression of C2orf18 and/or activity of C2orf18 protein. Expression of C2orf18 is inhibited, for example, by a double-stranded molecule that specifically target the C2orf18 gene. C2orf18 target sequences include, for example, nucleotide of SEQ ID NO: 7 or 8. In the present invention, the cells are characterized by over-expression of C2orf18, such as cancer cells, e.g. pancreatic cancer cells, specifically PDAC cells.

These modulating methods can be performed ex vivo or in vitro (e.g., by culturing the cell with the agent) or, alternatively, in vivo (e.g., by administering the agent to a subject). The methods involve administering a protein, or combination of proteins, or a nucleic acid molecule, or combination of nucleic acid molecules, as treatment to counteract aberrant expression of the C2orf18 genes or aberrant activity of their gene products.

Accordingly, agents that may be utilized in the context of the present invention include, e.g.

(i) a polypeptide encoded by C2orf18 gene or analogs, derivatives, fragments or homologs thereof;

(ii) antibodies to the C2orf18 gene or gene products, or fragment thereof;

(iii) antisense nucleic acids or nucleic acids that are “dysfunctional” (i.e., due to a heterologous insertion within the nucleic acids of C2orf 18 gene);

(iv) double-stranded molecules, e.g. small interfering RNAs (siRNAs); or

(v) modulators (i.e., inhibitors, antagonists that alter the interaction between a C2orf18 polypeptide and its binding partner).

The dysfunctional antisense molecules are utilized to “knockout” endogenous function of a polypeptide by homologous recombination (see, e.g., Capecchi, Science 1989, 244: 1288 92).

Increased levels can be readily detected by quantifying peptide and/or RNA in cells and assaying it in vitro for RNA or peptide levels, structure and/or activity of the expressed peptides (or mRNAs of a gene whose expression is altered). Methods that are well known within the art include, but are not limited to, immunoassays (e.g., by Western blot analysis, immunoprecipitation followed by sodium dodecyl sulfate (SDS) polyacrylamide gel electrophoresis, immunocytochemistry, etc.), RT-PCR and/or hybridization assays to detect expression of mRNAs (e.g., Northern assays, dot blots, in situ hybridization, etc.).

According to an aspect of the present invention, an agent screened through the present method may be used. Methods well known to those skilled in the art may be used to administer the agents. If said agent is encodable by a DNA, the DNA can be inserted into a vector for expressing the DNA and the vector administered to a cell. For introducing the vector of double-stranded molecule into the cell, transfection-enhancing agent can be used. FuGENE6 (Roche diagnostics), Lipofectamine 2000 (Invitrogen), Oligofectamine (Invitrogen), and Nucleofector (Wako pure Chemical) are useful as the transfection-enhancing agent.

Methods of Treating A C2orf18-Associating Disease:

Treating, ameliorating or preventing C2orf18 associating disease includes any of the following steps, such as surgical removal of C2orf18 overexpressing cells, inhibition of the growth of cancerous cells, involution or regression of a tumor, induction of remission and suppression of occurrence of cancer. Effective treating C2orf18 associating disease decreases mortality and improves the prognosis of individuals having C2orf18 associating disease, decreases the levels of tumor markers in the blood, and alleviates detectable symptoms accompanying C2orf18 associating disease.

Knockdown of endogenous C2orf18 by siRNA in the cell lines over-expressing C2orf18 gene resulted in drastic suppression of the cell growth, suggesting its essential role in maintaining viability of these cells. Therefore, the present invention relates to treating or preventing C2orf18 associating disease by inhibiting expression of C2orf18 and/or activity of C2orf18 protein. Expression of C2orf18 is inhibited, for example, by a double-stranded molecule that specifically target the C2orf 18 gene. C2orf 18 target sequences include, for example, nucleotide of SEQ ID NO: 7 or 8. Furthermore, the activity of C2orf18 is inhibited by an anti-C2orf18 antibody or peptide mimetics. In the present invention, the C2orf18 associating diseases are characterized by over-expression of C2orf18, such as cancer, e.g. pancreatic cancer, specifically PDAC.

In the present invention, the inhibitory nucleic acids can be administered to the subject either as a naked nucleic acid, in conjunction with a delivery reagent, or as a recombinant plasmid or viral vector which expresses the inhibitory nucleic acid. Suitable delivery reagents for administration in conjunction with the present inhibitory nucleic acids include the Mirus Transit TKO lipophilic reagent; lipofectin; lipofectamine; cellfectin; or polycations (e.g., polylysine), or liposomes. A preferred delivery reagent is a liposome.

Liposomes can aid in the delivery of the inhibitory nucleic acids to a particular tissue, such as retinal or tumor tissue, and can also increase the blood half-life of the inhibitory nucleic acids. Liposomes suitable for use in the invention are formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example as described in Szoka et al., Ann Rev Biophys Bioeng 1980, 9: 467; and U.S. Pat. Nos. 4,235,871; 4,501,728; 4,837,028; and 5,019,369, the entire disclosures of which are herein incorporated by reference.

Preferably, the liposomes encapsulating the present inhibitory nucleic acids include a ligand molecule that can deliver the liposome to the cancer site. Ligands which bind to receptors prevalent in tumor cells, such as monoclonal antibodies that bind to tumor antigens, are preferred.

Particularly preferably, the liposomes encapsulating the present inhibitory nucleic acids are modified so as to avoid clearance by the mononuclear macrophage and reticuloendothelial systems, for example, by having opsonization-inhibition moieties bound to the surface of the structure. In one embodiment, a liposome of the invention can include both opsonization-inhibition moieties and a ligand.

Opsonization-inhibition moieties for use in preparing the liposomes of the invention are typically large hydrophilic polymers that are bound to the liposome membrane. As used herein, an opsonization-inhibition moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibition hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016, the entire disclosure of which is herein incorporated by reference. Liposomes modified with opsonization-inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, target tissue characterized by such microvasculature defects, for example, solid tumors, will efficiently accumulate these liposomes; see Gabizon et al., Proc Natl Acad Sci USA 1988, 18: 6949-53. In addition, the reduced uptake by the RES lowers the toxicity of stealth liposomes by preventing significant accumulation in liver and spleen. Thus, liposomes of the invention that are modified with opsonization-inhibition moieties can deliver the present inhibitory nucleic acids to tumor cells.

Opsonization-inhibition moieties suitable for modifying liposomes are preferably water-soluble polymers with a molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as poly-acrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM.sub.1. Copolymers of PEG, methoxy PEG, or methoxy PPG, or derivatives thereof, are also suitable. In addition, the opsonization-inhibition polymer can be a block copolymer of PEG and either a polyamino acid, polysaccharide, polyamidoamine, polyethyleneamine, or polynucleotide. The opsonization-inhibition polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

Preferably, the opsonization-inhibition moiety is a PEG, PPG, or derivatives thereof.

Liposomes modified with PEG or PEG-derivatives are sometimes called “PEGylated liposomes”.

The opsonization-inhibition moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH. sub. 3 and a solvent mixture such as tetrahydrofuran and water in a 30:12 ratio at 60. degrees. C.

Vectors expressing inhibitory nucleic acids of the invention are discussed above. Such vectors expressing at least one inhibitory nucleic acids of the invention can also be administered directly or in conjunction with a suitable delivery reagent, including the Mirus Transit LT1 lipophilic reagent; lipofectin; lipofectamine; cellfectin; polycations (e.g., polylysine) or liposomes. Methods for delivering recombinant viral vectors, which express inhibitory nucleic acids of the invention, to an area of cancer in a subject are within the skill of the art.

The inhibitory nucleic acids of the invention can be administered to the subject by any means suitable for delivering the inhibitory nucleic acids into cancer sites. For example, the inhibitory nucleic acids can be administered by gene gun, electroporation, or by other suitable parenteral or enteral administration routes. Suitable enteral administration routes include oral, rectal, or intranasal delivery.

Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., peri-tumoral and intra-tumoral injection, intra-retinal injection, or subretinal injection); subcutaneous injection or deposition including subcutaneous infusion (such as by osmotic pumps); direct application to the area at or near the site of cancer, for example by a catheter or other placement device (e.g., a retinal pellet or a suppository or an implant having a porous, non-porous, or gelatinous material); and inhalation. It is preferred that injections or infusions of the inhibitory nucleic acids or vector be given at or near the site of cancer.

The inhibitory nucleic acids of the present invention can be administered in a single dose or in multiple doses. Where the administration of the inhibitory nucleic acids of the present invention is by infusion, the infusion can be a single sustained dose or can be delivered by multiple infusions. Injection of the agent directly into the tissue is at or near the site of cancer preferred. Multiple injections of the agent into the tissue at or near the site of cancer are particularly preferred.

One skilled in the art can also readily determine an appropriate dosage regimen for administering the inhibitory nucleic acids of the invention to a given subject. For example, the inhibitory nucleic acids can be administered to the subject once, for example, as a single injection or deposition at or near the cancer site. Alternatively, the inhibitory nucleic acids can be administered once or twice daily to a subject for a period of from about three to about twenty-eight days, more preferably from about seven to about ten days. In a preferred dosage regimen, the inhibitory nucleic acids are injected at or near the site of cancer once a day for seven days. Where a dosage regimen involves multiple administrations, it is understood that the effective amount of an inhibitory nucleic acids administered to the subject can include the total amount of an inhibitory nucleic acids administered over the entire dosage regimen.

Diseases and disorders that are characterized by increased (relative to a subject not suffering from the disease or disorder) expression levels or biological activities of genes and gene products, respectively, may be treated with therapeutics that antagonize (i.e., reduce or inhibit) activity of the over-expressed gene. Therapeutics that antagonize activity can be administered therapeutically or prophylactically. Accordingly, therapeutics that may be utilized in the context of the present invention include, e.g.

(i) a polypeptide encoded by C2orf 18 gene or analogs, derivatives, fragments or homologs thereof;
(ii) antibodies to the C2orf 18 gene or gene products, or fragment thereof;
(iii) antisense nucleic acids or nucleic acids that are “dysfunctional” (i.e., due to a heterologous insertion within the nucleic acids of over-expressed gene);
(iv) double-stranded molecules, e.g. small interfering RNAs (siRNAs); or
(v) modulators (i.e., inhibitors, antagonists that alter the interaction between an over-expressed polypeptide and its binding partner).

The dysfunctional antisense molecules are utilized to “knockout” endogenous function of a polypeptide by homologous recombination (see, e.g., Capecchi, Science 1989, 244: 1288 92).

Increased levels can be readily detected by quantifying peptide and/or RNA, by obtaining a subject tissue sample (e.g., from biopsy tissue) and assaying it in vitro for RNA or peptide levels, structure and/or activity of the expressed peptides (or mRNAs of a gene whose expression is altered). Methods that are well known within the art include, but are not limited to, immunoassays (e.g., by Western blot analysis, immuno-precipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

Prophylactic administration occurs prior to the manifestation of overt clinical symptoms of disease, such that a disease or disorder is prevented or, alternatively, delayed in its progression. In the context of the present invention, prevention is any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications. Accordingly, the present invention encompasses a wide range of prophylactic therapies aimed at alleviating the severity of C2orf18 associating disease, such as a cancer, e.g. pancreatic cancer, specifically PDAC.

Therapeutic methods of the present invention may include the step of contacting a cell with an agent that modulates one or more of the activities of the C2orf 18 gene products. Examples of agent that modulates protein activity include, but are not limited to, nucleic acids, proteins, naturally occurring cognate ligands of such proteins, peptides, peptidomimetics, and other small molecule.

Thus, the present invention provides methods for treating or alleviating a symptom of cancer, or preventing cancer in a subject by decreasing the expression of the C2orf18 gene or the activity of the gene product. The present method is particularly suited for treating or preventing C2orf18 associated disease, such as cancer, e.g. pancreatic cancer, specifically PDAC.

Suitable therapeutics can be administered prophylactically or therapeutically to a subject suffering from or at risk of (or susceptible to) developing C2orf18 associated disease. Such subjects can be identified by using standard clinical methods or by detecting an aberrant expression level (“up-regulation” or “over-expression”) of the C2orf 18 gene or aberrant activity of the gene product.

According to an aspect of the present invention, an agent screened through the present method may be used for treating or preventing cancer. Methods well known to those skilled in the art may be used to administer the agents to subjects, for example, as an intra-arterial, intravenous, or percutaneous injection or as an intranasal, trans-bronchial, intramuscular, or oral administration. If said agent is encodable by a DNA, the DNA can be inserted into a vector for gene therapy and the vector administered to a subject to perform the therapy. For introducing the vector of double-stranded molecule into the cell, transfection-enhancing agent can be used. FuGENE6 (Roche diagnostics), Lipofectamine 2000 (Invitrogen), Oligofectamine (Invitrogen), and Nucleofector (Wako pure Chemical) are useful as the transfection-enhancing agent.

The dosage and methods for administration vary according to the body-weight, age, sex, symptom, condition of the patient to be treated and the administration method; however, one skilled in the art can routinely select suitable dosage and administration method.

For example, although the dose of an agent that binds to the C2orf 18 polypeptide and regulates the activity of the polypeptide depends on the aforementioned various factors, the dose is generally about 0.1 mg to about 100 mg per day, preferably about 1.0 mg to about 50 mg per day and more preferably about 1.0 mg to about 20 mg per day, when administered orally to a normal adult human (60 kg weight).

When administering the agent parenterally, in the form of an injection to a normal adult human (60 kg weight), although there are some differences according to the patient, target organ, symptoms and methods for administration, it is convenient to intravenously inject a dose of about 0.01 mg to about 30 mg per day, preferably about 0.1 to about 20 mg per day and more preferably about 0.1 to about 10 mg per day. In the case of other animals, the appropriate dosage amount may be routinely calculated by converting to 60 kg of body-weight.

Similarly, a pharmaceutical composition of the present invention may be used for treating or preventing cancer. Methods well known to those skilled in the art may be used to administer the compositions to patients, for example, as an intra-arterial, intravenous, or percutaneous injection or as an intranasal, transbronchial, intramuscular, or oral administration.

For each of the aforementioned conditions, the compositions, e.g., polypeptides and organic compounds, can be administered orally or via injection at a dose ranging from about 0.1 to about 250 mg/kg per day. The dose range for adult humans is generally from about 5 mg to about 17.5 g/day, preferably about 5 mg to about 10 g/day, and most preferably about 100 mg to about 3 g/day. Tablets or other unit dosage forms of presentation provided in discrete units may conveniently contain an amount which is effective at such dosage or as a multiple of the same, for instance, units containing about 5 mg to about 500 mg, usually from about 100 mg to about 500 mg.

The dose employed will depend upon a number of factors, including the age, body weight and sex of the subject, the precise disorder being treated, and its severity. Also the route of administration may vary depending upon the condition and its severity. In any event, appropriate and optimum dosages may be routinely calculated by those skilled in the art, taking into consideration the above-mentioned factors.

In particular, an antisense nucleic acid against the C2orf18 gene can be given to the patient by direct application onto the ailing site or by injection into a blood vessel so that it will reach the site of ailment.

The dosage of the antisense nucleic acid derivatives of the present invention can be adjusted suitably according to the patient's condition and used in desired amounts. For example, a dose range of 0.1 to 100 mg/kg, preferably 0.1 to 50 mg/kg can be administered.

### Mode for the Invention 1

Hereinafter, the present invention is described in more detail with reference to the Examples. However, the following materials, methods and examples only illustrate aspects of the invention and in no way are intended to limit the scope of the present invention. As such, methods and materials similar or equivalent to those described herein can be used in the practice or testing of the present invention.

General Methods

1. Cell Lines and Clinical Samples

PDAC cell lines MIA-PaCa2 and Panc-1, and NIH3T3, HEK-293T and COST cells were purchased from ATCC (American Type Culture Collection). PDAC cell lines KLM-1, PK-59, PK-45P, SUIT-2, and PK-1 were provided by the Cell Resource Center for Biomedical Research, Tohoku University (Sendai, Japan). All cell lines were grown in RPMI-1640 (Invitrogen, Carlsbad, Calif.) for Panc-1, KLM-1, PK-59, PK-45P, SUIT-2, and PK-1, and Dulbecco's modified Eagle's medium (Invitrogen) for COS-7, NIH3T3, HEK-293, and MIA-PaCa2. All cell lines were grown in monolayers in appropriate media supplemented with 10% fetal bovine serum (FBS) and 1% antibiotic/antimycotic solution, and maintained at 37° C. in air containing 5% CO2. Frozen and paraffin-embedded PDAC tissues were obtained from surgical specimens that were resected in Osaka Medical Center for Cancer and Cardiovascular Diseases under the appropriate informed consent, and this study using these clinical samples were approved by IRB in Institute of Medical Science, The University of Tokyo, and Osaka Medical Center for Cancer and Cardiovascular Diseases.

2. Semi-Quantitative RT-PCR

The purification of PDAC cells and normal ductal epithelial cells from pancreatic cancer tissues was described previously (Nakamura T, et al. Oncogene. 2004 Mar. 25; 23(13):2385-400.). RNA from the purified PDAC cells and normal pancreatic ductal epithelial cells were subjected to two rounds of RNA amplification using T7-based in vitro transcription (Epicentre Technologies, Madison, Wis.) and synthesized to single-strand cDNA. Total RNA from human pancreatic cancer cell lines was extracted using Trizol reagent (Invitrogen) according to the manufacturer's recommended procedures. Extracted RNA was treated with DNase I (Roche Diagnostic, Basel, Switzerland) and reversely-transcribed to single-stranded cDNAs using oligo (dT) primer with Superscript II reverse transcriptase (Invitrogen). Appropriate dilutions of each single-stranded cDNA were prepared for subsequent PCR amplification by monitoring alpha-tubulin (TUBA) as a quantitative control. The following primer sequences were used;

(SEQ ID NO: 1)
5'-AAGGATTATGAGGAGGTTGGTGT-3'
and
(SEQ ID NO: 2)
5'-CTTGGGTCTGTAACAAAGCATTC-3'
for TUBA,
(SEQ ID NO: 3)
5'-GGTAGCTCAGTCATAAAACACCG-3'
and
(SEQ ID NO: 4)
5'- GTCTCTCCATCATCCTCACTGTC-3'
for C2orf18 (NM_017877).

All reactions involved initial denaturation at 94° C. for 2 min followed by 23 cycles (for TUBA) or 28 cycles (for C2orf18) at 94° C. for 30 s, 55° C. for 30 s, and 72° C. for 30 s, on a GeneAmp PCR system 9700 (PE Applied Biosystems, Foster, Calif.).

3. Northern Blot Analysis

Extracted total RNAs were extracted from 14 pancreatic cancer cell lines using Trizol reagent (Invitrogen) and performed Northern blot analysis. After treatment with DNase I (Nippon Gene), mRNA was purified with mRNA Purification Kit (GE Healthcare, Piscataway, N.J.), according to the manufacturer's protocols. One micro g of each mRNA from pancreatic cancer cell lines, as well as those isolated from normal human adult heart, lung, liver, kidney, bone marrow, and pancreas (BD Biosciences, Palo Alto, Calif.), were separated on 1% denaturing agarose gels and transferred onto a nylon membrane. This cancer membrane and Human Multiple Tissue blots (Clontech, Palo Alto, Calif.) were hybridized for 16 hours with ³²P-labeled C2orf 18 cDNA, which was labeled using a Mega Label kit (GE Healthcare). A 232-bp PCR product of C2orf 18 cDNA was prepared as a probe using primers 5′-GGTAGCTCAGTCATAAAACACCG-3′ (SEQ ID NO: 3) and 5′-GTCTCTCCATCATCCTCACTGTC-3′ (SEQ ID NO: 4). Pre-hybridization, hybridization, and washing were performed according to the manufacturer's instruction. The blots were autoradiographed at −80° C. for 10 days.

4. Generation of Antibodies Specific to C2orf18 Protein and Immunohistochemical Staining

The two peptides (CRAAGQSDSSVDPQQPF (SEQ ID NO: 5) and AEESEQERLLGGTRTPINDAS (SEQ ID NO: 6)) corresponding to the regions spanning between codon 70-86 and codon 351-371, respectively, of C2orf18 protein (Genbank accession no: NP_{—}060347, SEQ ID NO: 12) were generated by Sigma-Aldrich Japan (Ishikari, Japan) and their mixture was immunized to two rabbits. The immune sera were purified on affinity-columns packed with Affi-Gel 10 activated affinity media (Bio-Rad Laboratories, Hercules, Calif.) conjugating each of the peptide antigens with accordance of basic methodology. Conventional tissue sections from PDACs were obtained from surgical specimens that were resected in Osaka Medical Center for Cancer and Cardiovascular Diseases under the appropriate informed consent. The sections were deparaffinized and autoclaved at 108° C. in citrate buffer, pH 6.0 for 15 min. Endogenous peroxidase activity was quenched by incubation in Peroxidase Blocking Reagent (Dako Cytomation, Carpinteria, Calif.) for 30 min. After incubated with fetal bovine serum for blocking, the sections were incubated with rabbit anti-C2orf 18 polyclonal antibody (dilution 1:1500) at room temperature for 1 hour. After washing with PBS, immunodetection was performed with peroxidase labeled anti-rabbit immunoglobulin (Envision kit, Dako Cytomation). Finally, the reactants were developed with 3,3′-diaminobenzidine (Dako Cytomation). Counterstaining was performed using hematoxylin.

5. Small Interfering RNA (siRNA)-Expressing Constructs Specific to C2orf18

To down-regulate endogenous C2orf18 expression in PDAC cells, psiU6BX3.0 vectors were for expression of short hairpin RNA against a target gene as described previously (Taniuchi K, et al. Cancer Res. 2005 Jan. 1; 65(1):105-12.). The U6 promoter was cloned upstream of the gene-specific sequence (19-nt sequence from the target transcript, separated from the reverse complement of the same sequence by a short spacer, TTCAAGAGA), with five thymidines as a termination signal and a neo cassette for selection by Geneticin (Invitrogen). The target sequences for C2orf18 were 5′-GGAGCACAGCTTCCAGCAT-3′ (SEQ ID NO: 7) (#196), 5′-GCACGACAGTCAGCACAAG-3′ (SEQ ID NO: 8) (#574) and 5′-GTGACTTCCTCTTTATGGA-3′ (SEQ ID NO: 9) (#3254), and the target sequence for negative control was 5′-GAAGCAGCACGACTTCTTC-3′ (SEQ ID NO: 10) (siEGFP). The human PDAC cell lines, MIA-PaCa2 and Panc-1 cells, were plated on 10-cm dishes, and transfected with each of #196, #574, #3254, or siEGFP siRNAexpression vectors using FuGENE6 (Roche) according to manufacture's instruction. Cells were selected by 0.8 mg/ml (for MIA-Paca2), or 1.0 mg/ml (for Panc-1) Geneticin (Invitrogen). Cells were harvested at 7 days after transfection to analyze knockdown effect on C2orf18 by RT-PCR using the primers described above. After cultured in appropriate medium containing Geneticin for 9 days, the cells were fixed with 100% methanol, stained with 0.1% of crystal violet-H20 for colony formation assay.

In 3-(4,5-dimethylthiazol-2-yl)-2,5-diphenyltetrazolium bromide (MTT) assay, cell viability was measured using Cell-counting kit-8 (DOJINDO, Kumamoto, Japan) at 11 days after transfection. Absorbance was measured at 490 nm, and at 630 nm as reference, with a Microplate Reader 550 (Bio-Rad).sequences for siRNA

TABLE 1

| | | | SEQ | |
|---|---|---|---|---|
| | | | ID | posi- |
| siRNA | | sequence | NO | tion |
| #196 | target | GGAGCACAGCTTCCAGCAT | 7 | 196- |
| | | | | 214 |
| | insert F | CACCGGAGCACAGCTTCCAGCATTTC | 13 | |
| | | AAGAGAATGCTGGAAGCTGTGCTCC | | |
| | insert R | AAAAGGAGCACAGCTTCCAGCATTCT | 14 | |
| | | CTTGAAATGCTGGAAGCTGTGCTCC | | |
| | hairpin | GGAGCACAGCTTCCAGCATTTCAAGA | 15 | |
| | | GAATGCTGGAAGCTGTGCTCC | | |
| #574 | target | GCACGACAGTCAGCACAAG | 8 | 574- |
| | | | | 592 |
| | insert F | CACCGCACGACAGTCAGCACAAGTTC | 16 | |
| | | AAGAGACTTGTGCTGACTGTCGTGC | | |
| | insert R | AAAAGCACGACAGTCAGCACAAGTCT | 17 | |
| | | CTTGAACTTGTGCTGACTGTCGTGC | | |
| | hairpin | GCACGACAGTCAGCACAAGTTCAAGA | 18 | |
| | | GACTTGTGCTGACTGTCGTGC | | |
| #3254 | target | GTGACTTCCTCTTTATGGA | 9 | 3254- |
| | | | | 3272 |
| | insert F | CACCGTGACTTCCTCTTTATGGATTCA | 19 | |
| | | AGAGATCCATAAAGAGGAAGTCAC | | |
| | insert R | AAAAGTGACTTCCTCTTTATGGATCTC | 20 | |
| | | TTGAATCCATAAAGAGGAAGTCAC | | |
| | hairpin | GTGACTTCCTCTTTATGGATTCAAGAG | 21 | |
| | | ATCCATAAAGAGGAAGTCAC | | |
| #EGFP | target | GAAGCAGCACGACTTCTTC | 10 | |
| | insert F | CACCGAAGCAGCACGACTTCTTCTTC | 22 | |
| | | AAGAGAGAAGAAGTCGTGCTGCTTC | | |
| | insert R | AAAAGAAGCAGCACGACTTCTTCTCT | 23 | |
| | | CTTGAAGAAGAAGTCGTGCTGCTTC | | |
| | hairpin | GAAGCAGCACGACTTCTTCTTCAAGA | 24 | |
| | | GAGAAGAAGTCGTGCTGCTTC | | |

6. Immunocytochemical Analysis

Panc-1 cells were treated with RNA duplex corresponding to siC2orf18 (#196, #574) or siEGFP described above by using Lipofectamin 2000 or RNAiMAX (Invitrogen) according to the manufacture's recommended procedures, and 72 hours after the siRNA treatment, 200 nM MitoTracker Red (Invitrogen) was added to culture medium for 30 min, and the cells were fixed with 4% paraformaldehyde, and permeablilized with 0.1% Triton X-100 in PBS for lmin at room temperature. Non-specific binding was blocked by treatment with PBS containing 3% BSA for 30 min at room temperature. The cells were incubated for 60 min at room temperature with rabbit anti-C2orf18 antibody diluted in PBS containing 1% BSA (1:1000). After washing with PBS, the cells were stained by FITC-conjugated secondary antibody (Santa Cruz) for 60 min at room temperature. After washing with PBS, specimen was mounted with VECTASHIELD (VECTOR Laboratories, Inc, Burlingame, Calif.) containing 4′, 6′-diamidine-2′-phenylindolendihydrochrolide (DAPI) and visualized with Spectral Confocal Scanning Systems (Leica, Bensheim, Germany).

7. Cell Fractionation and Localization of C2orf18 Protein

To further investigate the subcellular localization of endogenous C2orf18 in cancer cells, the cell lysate of Panc-1 cells were first fractionated to separate the cytoplasmic fraction from the nuclear fraction by using Nuclear and Cytoplasmic Extraction Reagents (Pierce, Rockford, Ill.). Second, Panc-1 cells were homogenized in the homogenate buffer [0.25M sucrose, 10 mM Tris-HCl (pH 7.4), 1 mM EDTA] and ultra-centrifuged the cell lysate by 5000 rpm for 10 min at 4 degrees C. to remove the nuclei and debris. The supernatant was ultracentrifuged by 15000 rpm for 20 min at 4 degrees C. to collect the fraction of the mitochondria. The supernatant was ultracentrifuged again by 17000 rpm for 30 min at 4 degrees C. to separate the microsome fraction (supernatant) from other cytoplasmic components (pellet). Each of the cell fractions was separated on SDS-PAGE and detected the endogenous C2orf18 by anti-C2orf18 polyclonal antibody described above, and the mitochondria fraction was detected specifically by anti-mitofilin antibody (Abcam, Cambridge, UK).

8. Expression Constructs for C2orf18 and ANT2

Full-length cDNA encoding human C2orf18 (NM_{—}017877) was PCR-amplified by the use of a set of primers;

forward primer:
(SEQ ID NO: 27)
5′-ATTTGAGGAAGATCATGGCCTGGACCAAGTACCA-3′
and
reverse primer:
(SEQ ID NO: 28)
5′-CCGCTCGAGGCTGGCATCATTGATGGGA-3′.

Subsequently the cDNA was cloned into the Not1 and Xho1 sites of pCAGGS vector (pCAGGS-C2orf18-HA). Similarly, full-length cDNA fragment encoding human ANT2 (also referred as SLC25A5, SEQ ID NO: 25, GenBank™ Accession No. NM_{—}001152) was PCR-amplified by following primers; forward primer, 5′-ATTCGCGGCCGCTCATGACAGATGCCGCTGTGTC-3′ (SEQ ID NO: 29) and reverse primer, 5′-CCGCTCGAGTGTGTACTTCTTGATTTCA-3′ (SEQ ID NO: 30), and was cloned into the Not1 and Xho1 sites of pCAGGS vector (pCAGGS-ANT2-Flag). DNA sequences of these plasmids were confirmed by DNA sequencing.

9. Immunoprecipitation and Mass-Spectrometric Analysis for C2orf18-Interacting Proteins

To identify a protein(s) which could interact with C2orf 18, immunoprecipitation experiments were performed. The pCAGGS-C2orf 18-HA or empty pCAGGS-HA mock were transfected into pancreatic cancer cell line PK-1 using FuGENE6 (Roche). 48 hours after the transfection, the cells were collected and lysed in lysis buffer [50 mmol/L Tris-HCl (pH 8.0), 0.4% NP-40, 150 m mol/L NaCl, Protease Inhibitor Cocktail Set III (Calbiochem, San Diego, Calif.)]. Total proteins were incubated at 4° C. for 15 min with 17.5 micro g of rat monoclonal anti-HA antibody (Roche, clone3F10). Immuno-complexes were incubated with 300 micro 1 of protein G Sepharose (Zymed Laboratories, South San Francisco, Calif.) for 15 min and washed with lysis buffer. Co-precipitated proteins were separated in 12% SDS-PAGE gel and stained by silver-staining kit (Invitrogen). Bands that specifically appeared in the precipitates were excised with anti-HA antibody in PK-1 cells transfected with C2orf18-HA, but not in those in the cells transfected with the mock clone, and then digested them in-gel with trypsin and analyzed for peptide-mass fingerprints using an AXIMACFRMALDI-TOF mass spectrometer (Shimadzu Corp., Tsukuba, Japan). Peptide masses were searched with 10-ppm mass accuracy, and protein database searches were done using the database-fitting program IntelliMarque (Shimadzu). To confirm the interaction between C2orf18 and ANT2 proteins, C2orf18-HA expression vector and/or ANT2-Flag expression vector were co-transfected into COS-7 cells. The transfected cells were lysed as described above and immunoprecipitated with rat anti-HA antibody (Roche, clone3F10) or rabbit polyclonal anti-Flag antibody (Roche, F-7425). To examine interaction of C2orf18-HA and ANT-2-Flag proteins, these immune complexes were analyzed by western blotting with rabbit anti-FLAG or anti-HA antibodies.

10. Detection of the Mitochondrial Membrane Potential

KLM-1 cells were transfected with siRNA duplex targeting ANT2, 5′-GCAGATCACTGCAGATAA-3′ (SEQ ID NO: 31), C2orf18 siRNA duplex 5′-GGAGCACAGCTTCCAGCAT-3′ (#196/SEQ ID NO: 7) or siEGFP duplex 5′-GAAGCAGCACGACTTCTTC-3′ (as a control/SEQ ID NO: 10). 48 hours after the transfection, the cells were collected, and knockdown effect of siRNA duplex targeting C2orf 18 was confirmed by western blot analysis with anti-C2orf 18 polyclonal antibody as described above. The collected cells were washed twice with cold PBS, then incubated with 10 micro M Rhodamine-123 (Wako, Osaka, Japan) in PBS for 15 min at 37 degrees C. in the dark condition, washed with FACS buffer, and resuspended in 0.5 ml of FACS buffer containing 10 micro g/ml of propidium iodide (PI, Sigma-Aldrich, St. Louis, Mo.). Rhodamine-123 (Rh123) is known to accumulate into the mitochondria following the electrochemical gradient. Once the unincorporated Rh123 is removed, the incorporated Rh123 is preferentially retained in the mitochondria in an amount proportional to the mitochondrial membrane potential (Darzynkiewicz Z et al., Proc Natl Acad Sci USA 1981; 78: 2383-7). The loss of mitochondrial integrity of the opening of the permeability transition pore channel results in the leakage of this probe from the mitochondria and the consequent fluorescence decreases (Darzynkiewicz Z et al., Proc Natl Acad Sci USA 1981; 78: 2383-7, Johnson L V et al., Proc Natl Acad Sci USA 1980; 77: 990-994.). Fluorescence intensities from Rh123 (530 nm) and PI (600 nm) were measured by flow cytometry (FACSCalibur, BD).

11. TUNEL Assay.

Panc-1 cells were transfected with siRNA#196 duplex 5′-GGAGCACAGCTTCCAGCAT-3′ (SEQ ID NO: 7) and siEGFP duplex 5′-GAAGCAGCACGACTTCTTC-3′ (SEQ ID NO: 10) as a negative control. 72 hours after the transfection, the cells were collected, resuspended with PBS, and fixed by 1% paraformaldehyde in PBS (pH7.4) for 15 min. After washing by PBS, the cells were permeabilized by precooled ethanol+acetic acid (2:1) for 5 min at −20 degrees C. for immunocytochemical analysis, or by precooled 70% ethanol for flow cytometry. To detect apoptosis by TUNEL assay, the ApopTag Fluorescein Direct In situ Apoptosis Detection Kit (Millipore, Bedford, Mass.) was used according to the manufacturer's protocol, and visualized with Spectral Confocal Scanning Systems (Leica), or measured fluorescence by flow cytometry (Cell Lab Quanta SC MPL, Beckman Coulter, Fullerton, Calif.). The permeabilized cells treated with DNase I were prepared as positive controls for TUNEL assay.

### Mode for the Invention 2

Over-Expression of C2orf18 in PDAC Cells

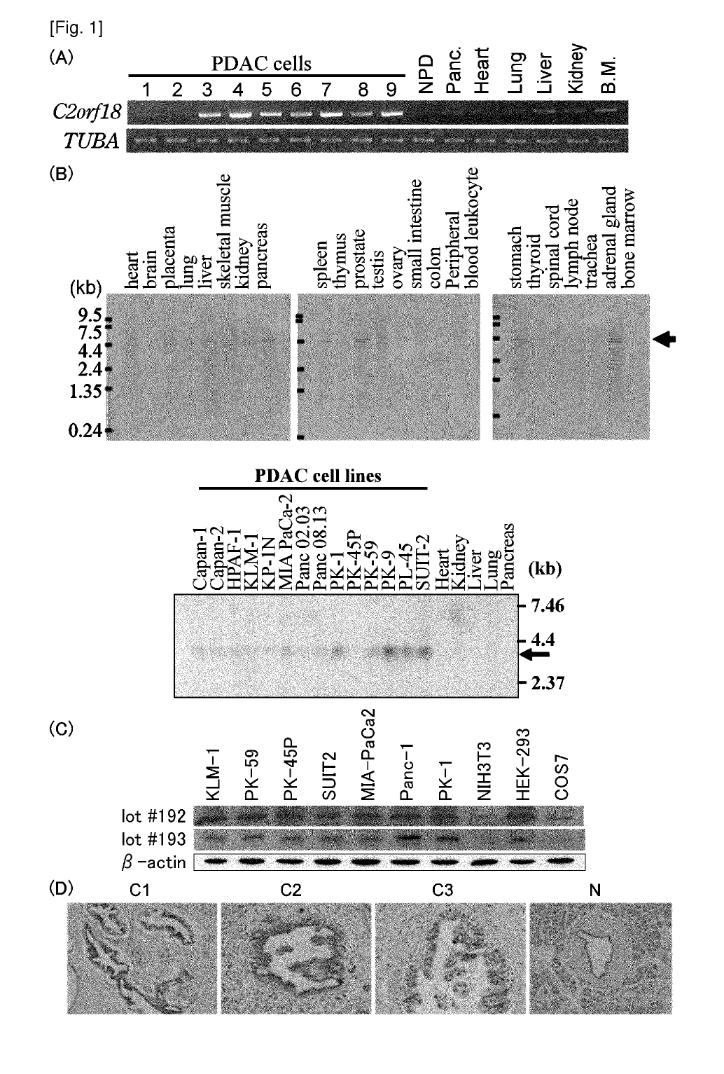

Among dozens of genes that were identified to be trans-activated in PDAC cells through our genome-wide cDNA microarray analysis (Nakamura T, et al. Oncogene. 2004 Mar. 25; 23(13):2385-400.4), one novel and uncharacterized gene, C2orf18, was selected for further study. This gene is referred to herein and elsewhere inter-changeably as ANT2BP (ANT2-binding protein) or PAMP (pancreas cancer mitochondrial protein). RT-PCR analysis confirmed C2orf18 over-expression in eight of the nine PDAC cells (FIG. 1A). Northern-blot analysis using a C2orf18 cDNA fragment as a probe identified about 4.4-kb transcript to be expressed in the prostate and the thyroid and showed that C2orf18 expression was detectable only faintly in vital organs including lung, heart, liver and kidney (FIG. 1B upperpanel). Its high level of expression was observed in most of PDAC cell lines examined (FIG. 1B lowerpanel).

Using a polyclonal antibody specific to C2orf18 protein, western blot analysis detected endogenous C2orf18 in several PDAC cell lines and normal cell lines (NIH3T3, HEK-293, and COST) and showed higher expression of C2orf18 in PDAC cell lines than normal cell lines (FIG. 1C). It was also performed immunohistochemical analysis on clinical PDAC tissue sections and found its strong staining in PDAC cells (FIG. 1D), but no staining in normal pancreas tissue (N in FIG. 1D). Totally, 10 out of 22 (45%) PDAC tissues showed positive staining for C2orf 18.

### Mode for the Invention 3

Effect of C2orf18-siRNAs on PDAC Cell Growth

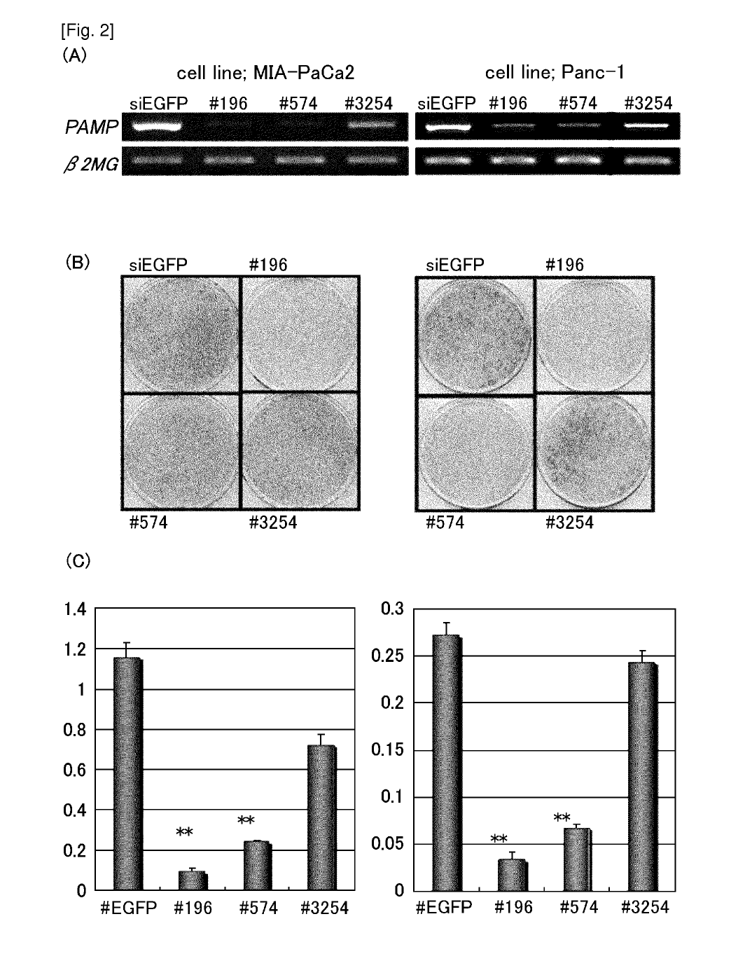

To investigate the biological function of C2orf 18 in PDAC cells and its potential as a molecular target for PDAC treatment, several siRNA-expression vectors specific to C2orf 18 were constructed and transfected into a PDAC cell line, MIA-PaCA2, that endogenously expressed high levels of C2orf18. RT-PCR indicated a significant knockdown effect of endogenous C2orf18 when it was transfected #196 and #574 siRNA-expression constructs (FIG. 2A). Colony-formation assays (FIG. 2B) and MTT assays (FIG. 2C) using #196 and #574 revealed a drastic reduction in the number of viable cells, compared to #3254 and a negative control siEGFP for which no knockdown effect was observed. In another C2orf18-positive PDAC cell line Panc-1, identical effects was observed (FIGS. 2A, B, and C).

### Mode for the Invention 4

Interaction of C2orf18 with ANT2 in the Mitochondria

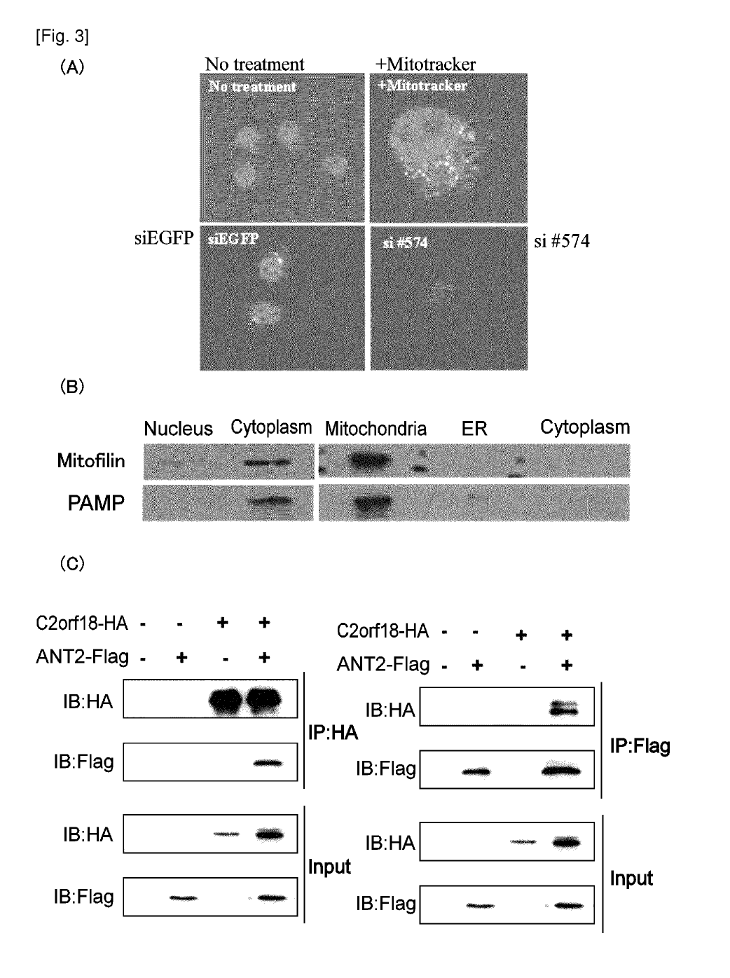

In-silico analysis predicted several trans-membrane domains in 371 amino-acid sequences of C2orf 18 protein. Immunocytochemical analysis using anti-C2orf 18 antibody revealed vesicular patterns of positive signals in the cytoplasm of PDAC cells and the disappearance of these fluorescent signals were observed by knockdown of endogenous C2orf 18 using siRNA duplex (FIG. 3A). These signals were partially merged with the signals of MitoTracker, the mitochondria-specific probe (FIG. 3A), indicating its localization in the mitochondria. To define the subcellular localization of C2orf 18 protein precisely, the lysate of Panc-1 cells was fractionated into the mitochondria, the endoplasmic reticulum (ER), and other cytoplasmic fraction. As shown in FIG. 3B, western blot analysis detected C2orf18 protein only in the mitochondrial fraction, as similar to the mitochondria-specific protein, mitofilin. In-silico analysis on C2orf 18 protein predicted its possible function as a permease related to nucleotide-sugar transporters. To investigate C2orf18 functions in cancer cells, a protein(s) interacting with C2orf18 was identified. Specifically, a protein complex including C2orf18 was immune-precipitated from the lysates of cells that over-expressed exogenous C2orf18-HA. A protein likely to interact with C2orf18 was characterized by mass spectrometry and the ANT2 protein was identified as a candidate interacting with C2orf18 protein. To validate the interaction between C2orf18 and ANT2, either of the vectors expressing C2orf18-HA, ANT2-Flag, or both vectors together were transfected into COS-7 cells, and a protein complex containing C2orf18-HA and/or ANT2-Flag was immunoprecipitated from the cell extracts by anti-HA antibody (FIG. 3C left) or anti-Flag antibody (FIG. 3C right). In FIG. 3C (left), western blot using anti-Flag antibody indicated that ANT2-Flag was co-immunoprecipitated with C2orf18-HA when the both expression vectors were co-transfected. Furthermore, in FIG. 3C (right), western blot using anti-HA antibody indicated that C2orf18-HA was co-immunoprecipitated with ANT2-Flag when the both expression vectors were co-expressed. Hence, this uncharacterized protein was termed an ANT2-binding protein (ANT2BP) and suspected its possible role as a nucleotide-sugar transporter as well as ANT2.

### Mode for the Invention 5

C2orf18/ANT2BP was Involved with the Mitochondrial Membrane Potential (Delta psi m) and Apoptosis

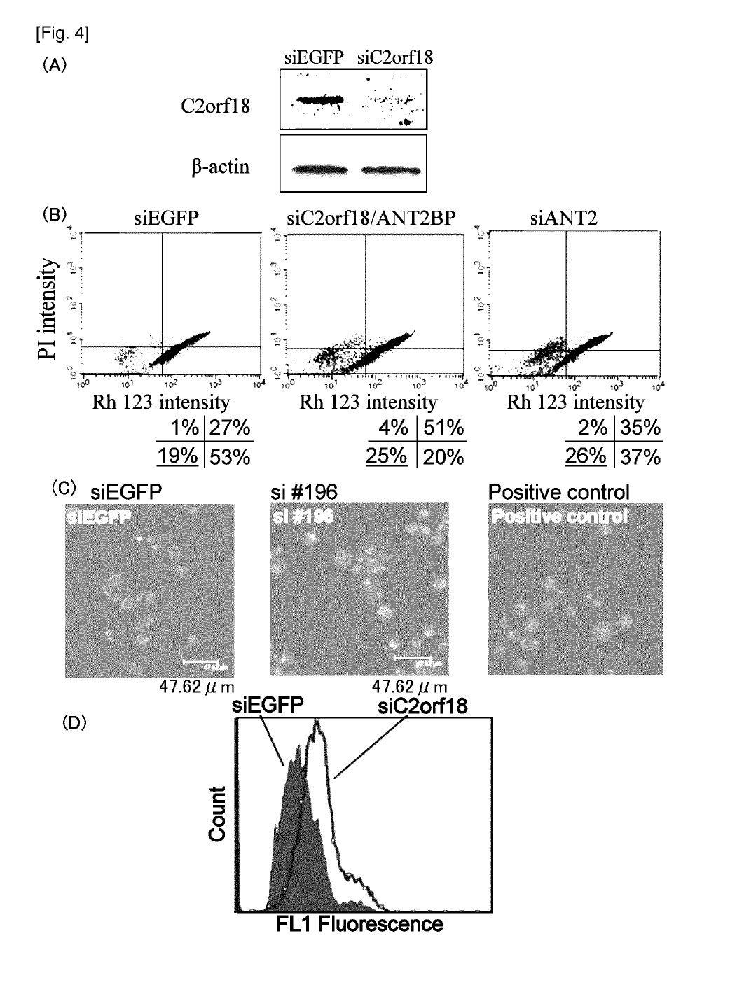

In cancer cells or cells with mitochondrial defect, the generation of mitochondrial membrane potential (delta psi m) is suspected to be dependent mainly on the ATP⁴⁻/ADP³⁻ exchange by ANT2 (Chevrollier A, et al. J Bioenerg Biomembr 2005; 37: 307-16., Bonod-Bidaud C, et al. Mitochondria 2001; 1: 217-224., Loiseau D, et al. Exp Cell Res 2002; 278: 12-18, Chevrollier A, et al. Mol Carcinog 2005; 42: 1-8.). Hence, ANT2 silencing facilitated apoptosis by modulating the mitochondrial membrane potential (Jang J Y, et al. Breast Cancer Res 2008; 10: R11., Le Bras M, et al. Cancer Res 2006; 66: 9143-52.). To examine whether ANT2BP could regulate the mitochondrial membrane potential delta psi m and be involved in mitochondrial apoptosis, ANT2BP expression was knocked down in PDAC cells and examined delta psi m and apoptosis by staining with Rhodamine-123 and TUNEL assay, respectively. Western blot analysis using anti-C2orf18/ANT2BP antibody confirmed knockdown effect of C2orf18/ANT2BP siRNA on KLM-1 cell (FIG. 4A). In FIG. 4B, Rhodamine-123 (Rh123) intensity at X-axis reflects delta psi m and PI (propidium iodide) permeability at Y-axis reflects the cell membrane destruction in dead cells. The low level of Rh123 intensity and negative-permeability of PI indicate the early apoptotic cells where delta psi m is decreased but apoptosis does not finish completely, while the low level of Rh123 intensity and positive-permeability of PI indicate dead cells (Shimizu S, et al. Oncogene 1996; 13: 21-9.). The numbers of the cells showing low delta psi m and negative-permeability of P1 were increased when ANTBP2 (25%) or ANT2 (26%) was knocked down, comparing with the control (siEGFP, 19%). This FACS analysis implicated that ANT2BP knockdown induced reduction of the mitochondrial membrane potential delta psi m, as well as ANT2 knockdown. Furthermore, TUNEL staining (FIG. 4C) and FACS analysis (FIG. 4D) showed a significant increase in the number of apoptosis cells when ANT2BP was knocked down. These data indicated ANT2BP could play a critical role in maintaining the mitochondrial membrane potential and be involved in the mitochondrial apoptotic pathway, as similar to ANT2, probably through its interaction with ANT2.

Discussion:

The evidence herein demonstrates one novel gene C2orf18/ANT2BP to be one of key molecules involved in pancreatic carcinogenesis through the genome-wide gene expression profile analysis of PDAC cells. Knockdown of C2orf18/ANT2BP using siRNA in PDAC cell lines resulted in drastic suppression of cancer cell viability and induced apoptosis following the breakdown of the mitochondrial membrane potential, implicating its essential role in maintaining viability of PDAC cells.

The evidence also indicates an interaction between C2orf 18 and ANT2, which was shown to have a function to catalyze the exchange of mitochondrial ADP with cytosolic ATP as one of the component of mitochondrial permeability transition pore complex (PTPC). PTPC plays a key role in regulation of the mitochondrial membrane permeabilization during apoptosis, necrosis and autophagy, and ANT family members have critical roles in maintenance of the mitochondrial membrane potential as well as ATP exchange or metabolism related with respiration (Crompton M. J Physiol 2000; 529: 11-21., Verrier F, et al. Oncogene 2004; 23: 8049-64.). In cancer cells, energy metabolism or ATP production is dependent mainly on glycolysis, which has been recognized as Warburg effect (Wallace D E. Cold Spring Harb Symp Quant Biol 2005; 70:363-74.). Among ANT family members, only ANT2 is likely to be associated with this glycolysis-dependent ATP production and its transport, and was shown to be upregulated in cancer cells and proliferating cells under hypoxia as well as cells with mitochondrial defect (Chevrollier A, et al. J Bioenerg Biomembr 2005; 37: 307-16., Bonod-Bidaud C, et al. Mitochondria 2001; 1: 217-224., Loiseau D, et al. Exp Cell Res 2002; 278: 12-18, Chevrollier A, et al. Mol Carcinog 2005; 42: 1-8.). Since it interacts with ANT2, C2orf18/ANT2BP is suspected to be involved in glycolysis-dependent ATP production and transport as a member of the ANT2 complex and make pancreatic cancer cells resistant to hypoxia or chemotherapy, although further functional analysis of C2orf18/ANT2BP in the mitochondria or energy metabolism of cancer cells is required. Some small compounds are already established to inhibit an ANT transporter as anti-cancer drugs (Galluzzi L, et al. Oncogene 2006; 25: 4812-30., Don A S, et al. Cancer Cell 2003; 3: 497-509., Machida K, et al. J Bio Chem 2002; 277: 31243-31248.). Considering the possible transporter function of C2orf18/ANT2BP which was predicted by in-silico analysis and was supported by the data herein, further functional analysis could lead to development of an inhibitor to C2orf18/ANT2BP as a novel anti-cancer drug. In summary, the expressional and functional findings herein suggest that C2orf18/ANT2BP is a promising molecular target for PDAC therapy and other cancers.

### INDUSTRIAL APPLICABILITY

Gene expression analyses of pancreatic cancer, particularly pancreatic ductal adenocarcinoma (PDAC) discussed herein, obtained through a combination of laser-capture dissection and genome-wide cDNA microarray, identified the C2orf18 gene as a novel target for pancreatic cancer detection, diagnosis and therapy. Based on the expression of C2orf18, the present invention provides molecular diagnostic markers for identifying and detecting pancreatic cancer, particularly pancreatic ductal adenocarcinoma (PDAC).

The methods described herein are also useful in the identification of additional molecular targets for detection, diagnosis, treatment and prevention of pancreatic cancers such as PDAC. The data reported herein add to a comprehensive understanding of pancreatic cancer, to thereby facilitate the development of novel diagnostic strategies, and provide clues for identification of molecular targets for therapeutic drugs and preventative agents. Such information contributes to a more profound understanding of pancreatic tumorigenesis, and provides indicators for developing novel strategies for detection, diagnosis, treatment, and ultimately prevention of pancreatic cancer.

Furthermore, the methods described herein are also useful in diagnosis of pancreatic cancer such as PDAC, as well as the monitoring the progress and prognosis of the patients with this disease. Moreover, the data reported here is also provide a likely candidate for development of therapeutic approaches for pancreatic cancers such as PDAC.

All publications, patent applications, patents and other references mentioned herein are incorporated by reference in their entirety. However, nothing herein should be construed as an admission that the invention is not entitled to antedate such disclosure by virtue of prior invention.

While the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Further advantages and features will become apparent from the claims filed hereafter, with the scope of such claims to be determined by their reasonable equivalents, as would be understood by those skilled in the art. Thus, the invention is intended to be defined not by the above description, but by the following claims and their equivalents.

1-14. (canceled)

15. A method of identifying an agent that inhibits the binding between C2orf18 and ANT2, said method comprising the steps of:
a) contacting a first polypeptide selected from the group consisting of:
i) a polypeptide comprising the amino acid sequence of SEQ ID NO: 12;
ii) a polypeptide comprising the amino acid sequence of SEQ ID NO: 12, wherein one or more amino acids are added, substituted, deleted, or inserted, provided the polypeptide has a binding activity to ANT2 equivalent to that of a polypeptide consisting of the amino acid sequence of SEQ ID NO: 12;
iii) a polypeptide comprising an amino acid sequence that is at least about 80% homologous to a polypeptide consisting of the amino acid sequence of SEQ ID NO: 12, provided the polypeptide has a binding activity to ANT2 equivalent to that of a polypeptide consisting of the amino acid sequence of SEQ ID NO: 12; and
vi) a polypeptide encoded by a polynucleotide that hybridizes under stringent conditions to a polynucleotide consisting of the nucleotide sequence of SEQ ID NO: 11, provided the polypeptide has the binding activity to ANT2 equivalent to that of a polypeptide consisting of the amino acid sequence of SEQ ID NO: 12;
in the presence of an agent with a second polypeptide selected from the group consisting of:
i) a polypeptide comprising the amino acid sequence of SEQ ID NO: 26;
ii) a polypeptide comprising the amino acid sequence of SEQ ID NO: 26 wherein one or more amino acids are added, substituted, deleted, or inserted, provided the polypeptide has a binding activity to C2orf18 equivalent to that of a polypeptide consisting of the amino acid sequence of SEQ ID NO: 26;
iii) a polypeptide comprising the amino acid sequence that has at least about 80% homology to a polypeptide consisting of the amino acid sequence of SEQ ID NO: 26, provided the polypeptide has a binding activity to C2 orf18 equivalent to that of a polypeptide consisting of the amino acid sequence of SEQ ID NO: 26; and
vi) a polypeptide encoded by a polynucleotide that hybridizes under stringent conditions to a polynucleotide consisting of the nucleotide sequence of SEQ ID NO: 25, provided the polypeptide has the binding activity to C2 orf18 equivalent to that of a polypeptide consisting of the amino acid sequence of SEQ ID NO: 26;
b) detecting the level of binding between the first polypeptide and the second polypeptide;
c) comparing the binding level of the first and second polypeptides with that detected in the absence of the agent; and
d) selecting the agent that reduces the binding level between the first and second polypeptides.

16. A method of inhibiting cancer cell growth in a subject comprising the step of administering to said subject a double-stranded molecule, wherein said double-stranded molecule reduces the expression level of C2orf18.

17. The method of claim 16, wherein said double-stranded molecule comprises a sense nucleic acid and an anti-sense nucleic acid of C2orf18.

18-19. (canceled)

20. The method of claim 16, wherein said double-stranded molecule is administered with a transfection-enhancing agent to a subject.

21. The method of claim 16, wherein said cancer is pancreatic cancer.

22. A composition for treating or preventing cancer, said composition comprising a pharmaceutically effective amount of a double-stranded molecule against a C2 orf18 as an active ingredient, and a pharmaceutically acceptable carrier.

23-24. (canceled)

25. A composition for treating or preventing cancer comprising a pharmaceutically effective amount of a vector encoding a double-stranded molecule against a C2orf18 as an active ingredient, and a pharmaceutically acceptable carrier.

26. The composition of claim 22, wherein said cancer is pancreatic cancer.

27. A double-stranded molecule comprising a sense strand and an antisense strand, wherein the sense strand comprises a nucleotide sequence corresponding to a target sequence consisting of SEQ ID NO: 7 or 8, and wherein the antisense strand comprises a nucleotide sequence which is complementary to said sense strand, wherein said sense strand and said antisense strand hybridize to each other to form said double-stranded molecule, and wherein said double-stranded molecule, when introduced into a cell expressing the C2 orf18 gene, inhibits expression of said gene.

28. The double-stranded molecule of claim 27, wherein said sense strand is from about 19 to about 25 nucleotides in length.

29. The double-stranded molecule of claim 28, wherein said double-stranded molecule is a single nucleotide transcript comprising the sense strand and the antisense strand linked via a single-stranded nucleotide sequence.

30. The double-stranded molecule of claim 29, wherein said double-stranded molecule has the general formula 5′-[A]-[B]-[A′]-3′, wherein [A] is a nucleotide sequence consisting of SEQ ID NO: 7 or 8; [B] is a nucleotide sequence consisting of about 3 to about 23 nucleotides; and [A′] is a nucleotide sequence complementary to [A].

31. A vector comprising each or both of a combination of polynucleotide comprising a sense strand nucleic acid and an antisense strand nucleic acid, wherein said sense strand nucleic acid comprises a nucleotide sequence of SEQ ID NO: 7 or 8, and said antisense strand nucleic acid comprises a sequence complementary to said sense strand, wherein the transcripts of said sense strand and said antisense strand hybridize to each other to form a double-stranded molecule, and wherein said vector, when introduced into a cell expressing the C2orf18 gene, inhibits the cell proliferation.

32. Vectors comprising each of a combination of polynucleotide comprising a sense strand nucleic acid and an antisense strand nucleic acid, wherein said sense strand nucleic acid comprises nucleotide sequence of SEQ ID NO: 7 or 8, and said antisense strand nucleic acid comprises of a sequence complementary to the sense strand, wherein the transcripts of said sense strand and said antisense strand hybridize to each other to form a double-stranded molecule, and wherein said vectors, when introduced into a cell expressing the C2orf18 gene, inhibits the cell proliferation.

33. The vector of claim 31, wherein the transcript further comprises a single-stranded nucleotide sequence linking said sense strand and said antisense strand.

34. The vector of claim 33, wherein said polynucleotide has the general formula 5′-[A]-[B]-[A′]-3′, wherein [A] is a nucleotide sequence consisting of SEQ ID NO: 7 or 8; [B] is a nucleotide sequence consisting of about 3 to about 23 nucleotides; and [A′] is a nucleotide sequence complementary to [A].

35-37. (canceled)

38. A method of inhibiting cancer cell growth in a subject comprising the step of administering to said subject a double-stranded molecule, wherein said double-stranded molecule reduces the expression level of C2orf18,
wherein said double-stranded molecule is the double-stranded molecule of claim 27.

39. A composition for treating or preventing cancer, said composition comprising a pharmaceutically effective amount of a double-stranded molecule against a C2orf18 as an active ingredient, and a pharmaceutically acceptable carrier,
wherein said double-stranded molecule is the double-stranded molecule of claim 27.

40. The composition of claim 25, wherein said cancer is pancreatic cancer.
